(19) United States
(12) Patent Application Publication
(10) Pub. No.: US 2024/0368453 A1
(43) Pub. Date: Nov. 7, 2024

(54) ULTRA-SHORT HYDROPHOBE ALKOXYLATES AS SURFACTANTS

(71) Applicants: ULTIMATE EOR SERVICES LLC, Austin, TX (US); HARCROS CHEMICALS, INC., Kansas City, KS (US)

(72) Inventors: Upali Peter WEERASOORIYA, Austin, TX (US); Don Pathma Jith LIYANAGE, Pflugerville, TX (US); Karasinghe Arachchige Nadeeka UPAMALI, Austin, TX (US); Christopher James BRITTON, Leander, TX (US); Robert Matthew DEAN, Austin, TX (US); John BOOREM, Kansas City, KS (US); Kurt CHESHIRE, Shawnee, KS (US)

(21) Appl. No.: 18/687,026

(22) PCT Filed: Aug. 29, 2022
(86) PCT No.: PCT/US2022/041939

§ 371 Date: Feb. 27, 2024

Related U.S. Application Data
(60) Provisional application No. 63/237,888, filed on Aug. 27, 2021.

(51) Int. Cl. C09K 8/584; C09K 8/588

(52) U.S. Cl. CPC C09K 8/584; C09K 8/588

(57) ABSTRACT

Disclosed are surfactants, as well as methods of using thereof in oil and gas operations.

### CROSS-REFERENCE TO RELATED APPLICATIONS

This application claims benefit of U.S. Provisional Application No. 63/237,888, filed Aug. 27, 2021, which is hereby incorporated herein by reference in its entirety.

### BACKGROUND

Enhanced Oil Recovery (EOR) refers to techniques for increasing the amount of unrefined petroleum, or crude oil that may be extracted from an oil reservoir (e.g., an oil field). Using EOR, 40-60% of the reservoir's original oil can typically be extracted compared with only 20-40% using primary and secondary recovery (e.g., by water injection or natural gas injection). Enhanced oil recovery may also be referred to as improved oil recovery or tertiary oil recovery (as opposed to primary and secondary oil recovery).

Enhanced oil recovery may be achieved by a variety of methods including miscible gas injection (which includes carbon dioxide flooding), chemical injection (which includes polymer flooding, alkaline flooding, and surfactant flooding), microbial injection, or thermal recovery (which includes cyclic steam, steam flooding, and fire flooding). The injection of various chemicals, usually as dilute aqueous solutions, has been used to improve oil recovery. Injection of alkaline or caustic solutions into reservoirs with oil that has organic acids naturally occurring in the oil (also referred to herein as “unrefined petroleum acids”) will result in the production of soap that may lower the interfacial tension enough to increase production. Injection of a dilute solution of a water soluble polymer to increase the viscosity of the injected water can increase the amount of oil recovered from geological formations. Aqueous solutions of surfactants such as petroleum sulfonates may be injected to lower the interfacial tension or capillary pressure that impedes oil droplets from moving through a reservoir. Special formulations of oil, water and surfactant microemulsions have also proven useful. Such formulations often include co-solvent compounds to increase the solubility of the solutes in the presence of oil and decrease the viscosity of an emulsion.

There is a need in the art for cost effective methods for enhanced oil recovery using chemical injection. Provided herein are methods and compositions addressing these and other needs in the art.

### SUMMARY

Provided herein are compounds, compositions, and methods for enhanced oil recovery. A compound described herein can be defined by Formula I

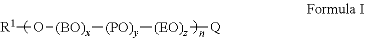

wherein

BO represents —CH₂—CH(ethyl)-O— or —CH₃CH(O—)CH₃;

PO represents —CH₂—CH(methyl)-O—;

EO represents —CH₂—CH₂—O—;

R¹ represents a C₁-C₆ alkyl group or a cyclic structure derived from alkoxylation of an alkyl monoglucoside, an alkyl polyglucoside, a monosaccharide, a disaccharide, or a polysaccharide;

n is an integer from 2 to 6;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation;

x is an integer from 0 to 15;

y is an integer from 11 to 60; and

z is an integer from 0 to 100.

In some embodiments, n can be an integer from 3 to 6. In some embodiments, n can be 3. In some embodiments, n can be 4. In some embodiments, y can be an integer from 11 to 40. In some embodiments, R¹ can be a C₃-C₆ alkyl group. In some embodiments, when Q is hydrogen, z is at least 6. In some embodiments, Q is —SO₃M⁺, —SO₃H, —CH₂C(O)O⁻M⁺, or —CH₂C(O)OH; and M⁺ is a cation. In some embodiments, z can be greater than 1. In some embodiments, z can be an integer from 5 to 75. In some embodiments, x can be 0. In some embodiments, compound can be defined by Formula II below

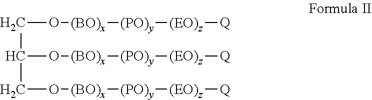

wherein

BO represents —CH₂—CH(ethyl)-O— or —CH₃CH(O—)CH₃;

PO represents —CH₂—CH(methyl)-O—;

EO represents —CH₂—CH₂—O—;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation;

x is an integer from 0 to 15;

y is an integer from 11 to 60; and

z is an integer from 0 to 100.

In some embodiments, the compound can defined by Formula III below

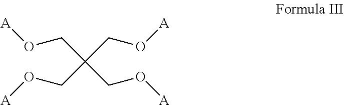

wherein

A represents —(BO)ₓ—(PO)_{y}-(EO)_{z}-Q

BO represents —CH₂—CH(ethyl)-O— or —CH₃CH(O—)CH₃;

PO represents —CH₂—CH(methyl)-O—;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation;

x is an integer from 0 to 15;

y is an integer from 11 to 60; and

z is an integer from 0 to 100.

Described herein are also compositions including a compound described herein and water. In some embodiments, the compound can be present in the composition in an amount of from 0.05% to 2% by weight, based on the total weight of the composition. In some embodiments, the composition can further include an additional surfactant. In some embodiments, the additional surfactant comprises an anionic surfactant, a non-ionic surfactant, a cationic surfactant, a zwitteronic surfactant, or any combination thereof. In some embodiments, the additional surfactant is present in the composition in an amount of from 0.05% to 2% by weight, based on the total weight of the composition. In some embodiments, the composition can further include a viscosity-enhancing water-soluble polymer. In some embodiments, the composition can further include an alkali agent. In some embodiments, the composition has a pH of from 10 to 12. In some embodiments, the composition can further include a co-solvent. In some embodiments, the composition can have a salinity of at least 5,000 ppm.

Described herein are also methods of displacing an unrefined petroleum material in contact with a solid material, said method comprising:

contacting the unrefined petroleum material with the compound described herein or a composition described herein, wherein the unrefined petroleum material is in contact with the solid material; and

allowing the unrefined petroleum material to separate from the solid material, thereby displacing the unrefined petroleum material in contact with the solid material.

The details of one or more embodiments of the disclosure are set forth in the accompanying drawings and the description below. Other features, objects, and advantages of the disclosure will be apparent from the description and drawings, and from the claims.

The surfactants described herein are further described in the attached Appendix, which is hereby incorporated by reference in its entirety.

### DESCRIPTION OF DRAWINGS

FIG. 1 shows a graph of salinity versus oil concentration when using 1% Phenol-1PO-10EO solvent at ˜1.5% Na2CO3 with 10%, 30%, and 50% oil.

FIG. 2 shows a graph of salinity versus oil concentration when using 1% glycerine-6PO co-solvent.

FIG. 3 shows a graph of salinity versus oil concentration when using 1% glycerine-12PO co-solvent.

FIG. 4 shows a graph of salinity versus oil concentration when using 1% glycerine-25PO co-solvent.

FIG. 5 shows a graph of salinity versus oil concentration when using 0.25% glycerine-12PO co-solvent.

FIG. 6 shows a graph of salinity versus oil concentration when using 1% glycerine-25PO co-solvent.

FIG. 7 shows a graph of salinity versus oil concentration when using 0.5% glycerine-25PO co-solvent.

FIG. 8 shows a graph of salinity versus oil concentration when using 0.25% glycerine-25PO co-solvent.

FIG. 9 shows a graph of salinity versus oil concentration when using 0.1% glycerine-25PO co-solvent.

FIG. 10 shows an image when using 0.1% glycerine-25PO co-solvent with 10% oil.

FIG. 11 shows an image of front-end dilution with PF showing Ultra Low IFT (UL-IFT) mixing with hard water including polymer.

FIG. 12 shows an image of back-end dilution with PD in soft brine showing UL-IFT mixing and going to type I at the end of the PD.

FIG. 13 shows left-blend of sulfonate with MEA solid paste. right-blend of glycerin-30PO-35EO with MEA, flowable liquid at 8° C.

FIG. 14 shows a coreflood setup.

FIG. 15 shows images of phase behavior tubes with 10%, 30%, and 50% oil as a function of salinity. Brine contained 1% MEA with 0.5% glycerin-30PO-35EO co-solvent. Samples were equilibrated at 22° C. for two weeks.

FIG. 16 shows graphical representation (activity map) of the phase behavior tubes shown in FIG. 3. Alkali consumption determination via single phase flood: MEA versus Na₂CO₃ in reservoir rock.

FIG. 17 shows coreflood results with oil recovery, oil saturation, oil cut and normalized produced cosolvent concentration on the left y-axis. Pressure drop is shown on right y-axis.

FIG. 18 shows normalize produced OH⁻ and comparative normalized KCl tracer versus pore volume.

FIG. 19 shows images of effluent from tubes 11-20.

FIG. 20 shows images showing zero Mg(OH)₂ precipitation with hard brine in the presence of 0.5% MEA.

FIG. 22 shows the activity map constructed from the data generated using formulation 1: 1% MEA and 0.5% glycerine-30PO-35EO-OH, mixing Brine A and B at 21° C.

FIG. 21 shows the structure of example USH-based alkoxylate surfactants and co-solvents, amine-based polyol alkoxylate surfactants and co-solvents, and glycerol-based alkoxylate surfactants and co-solvents.

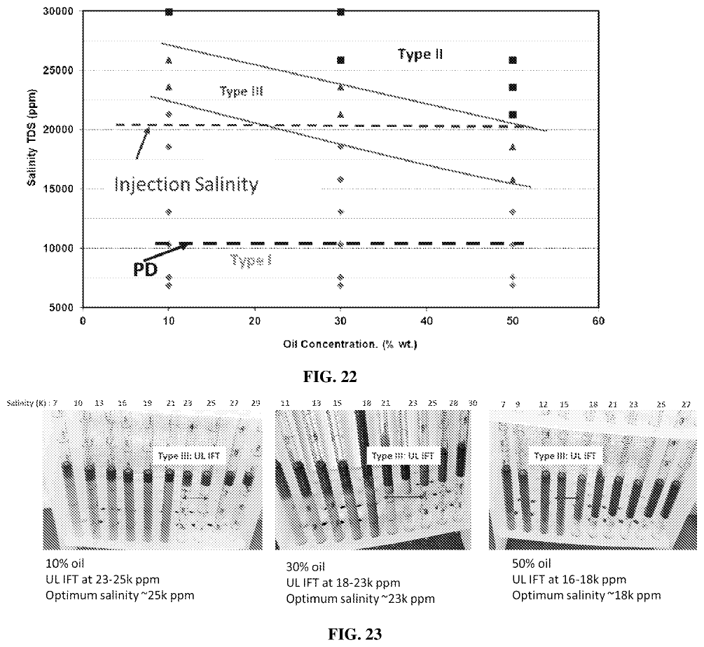

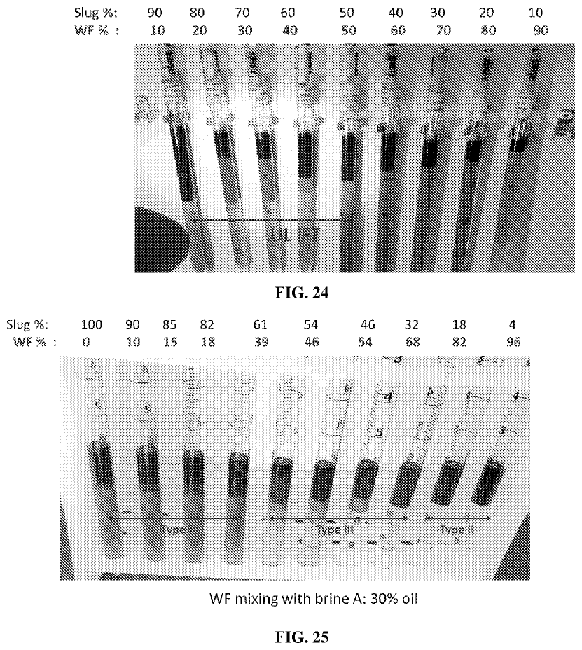

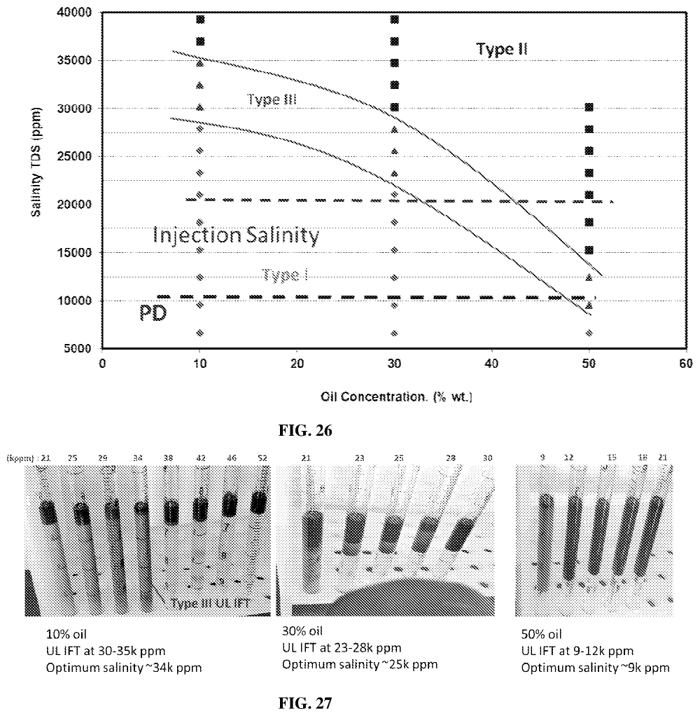

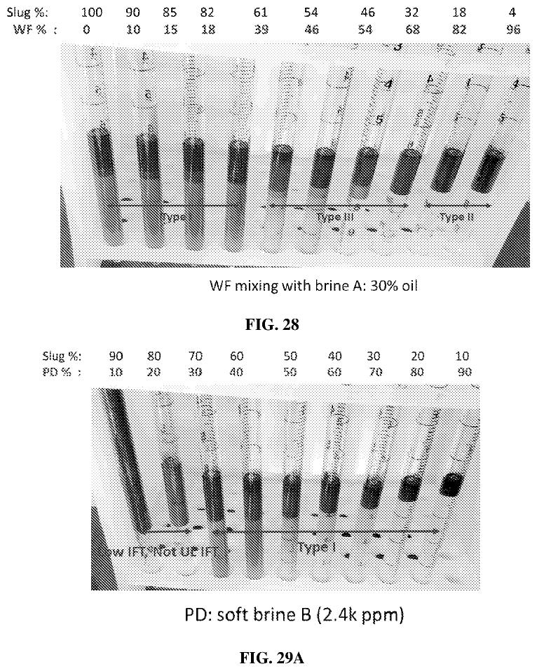

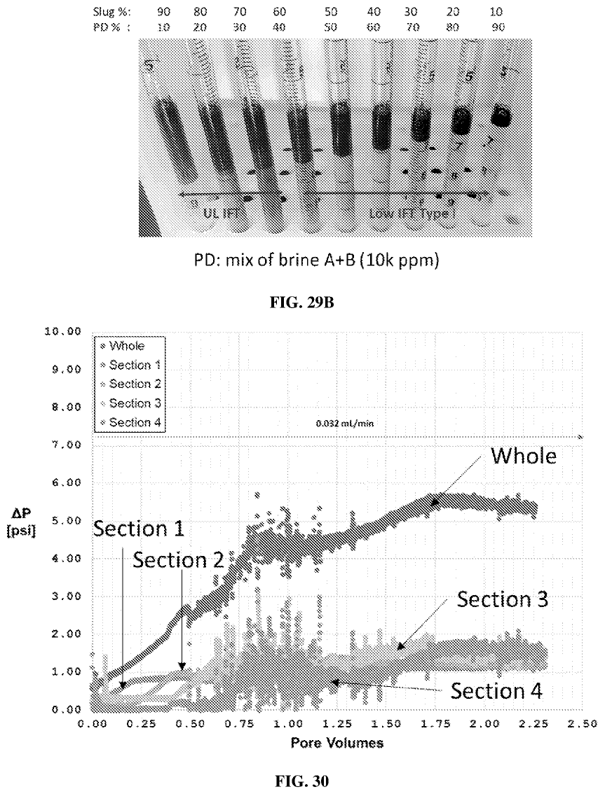

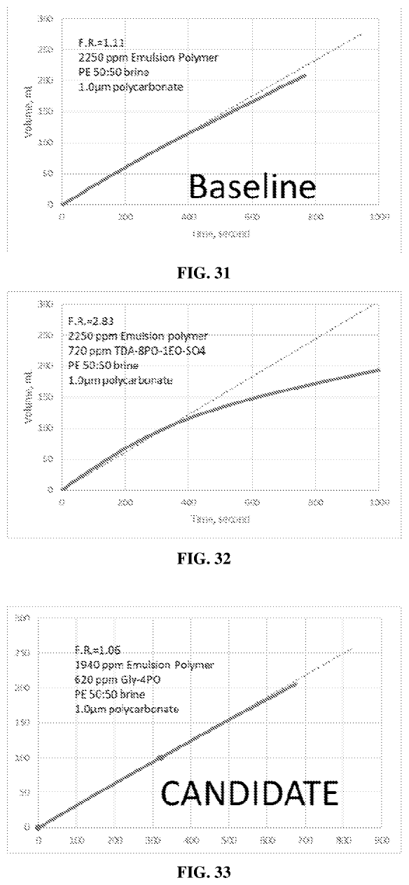

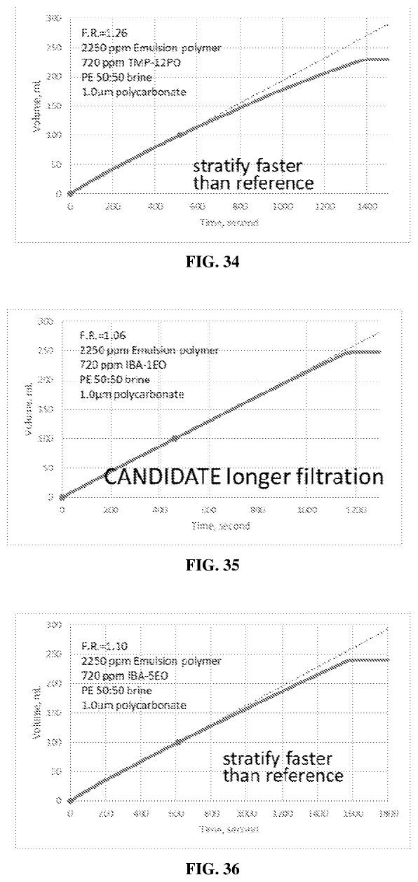

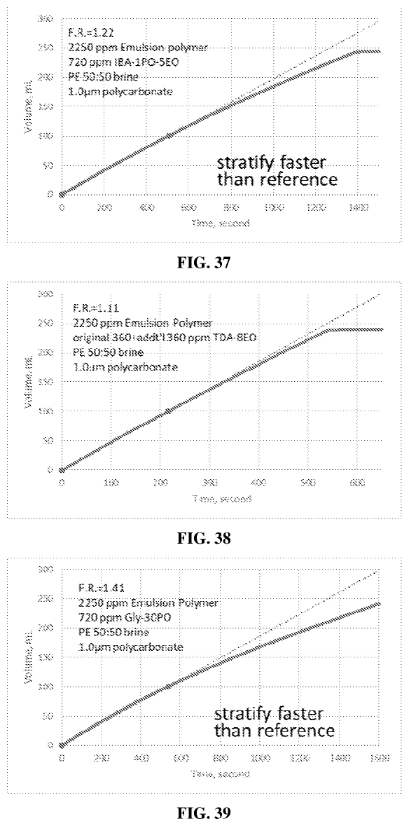

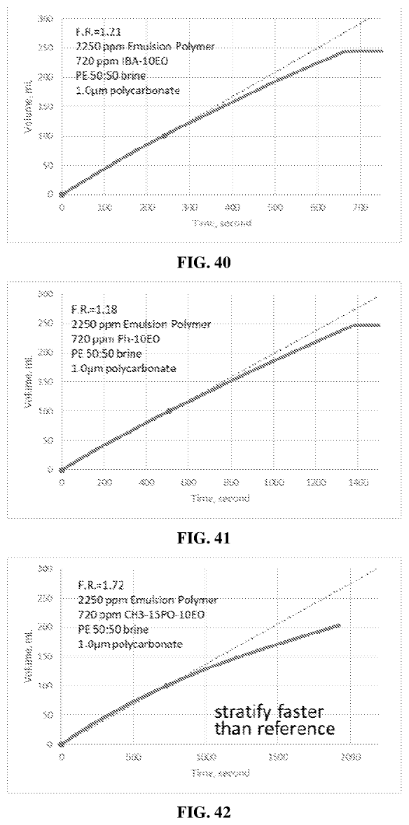

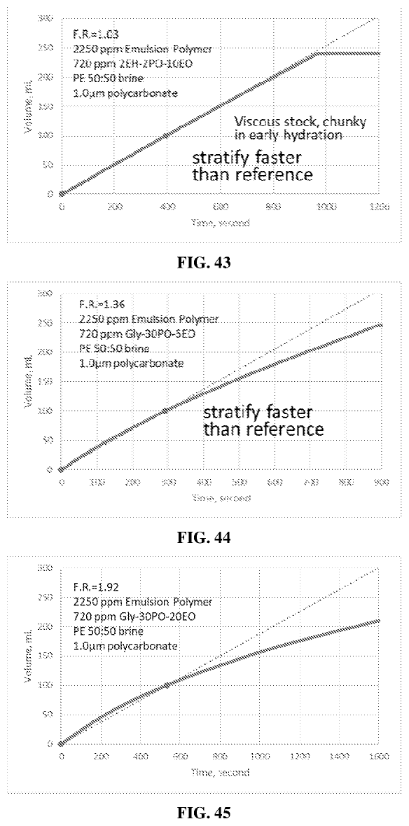

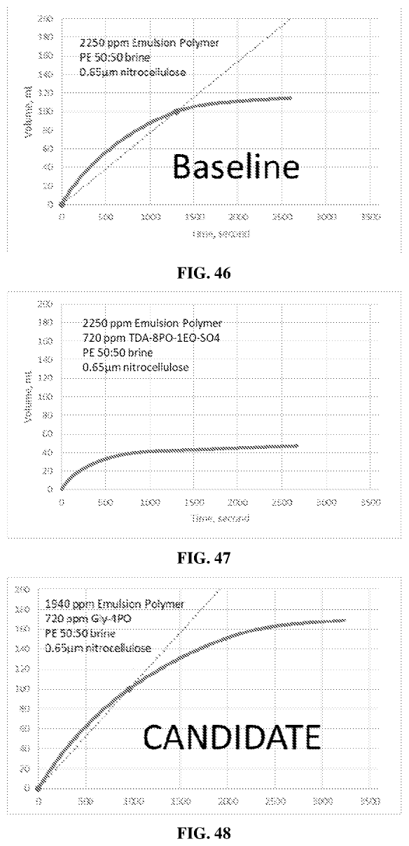

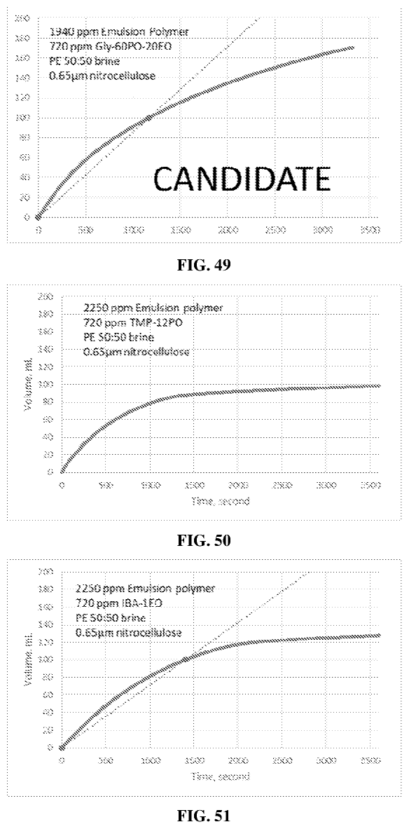

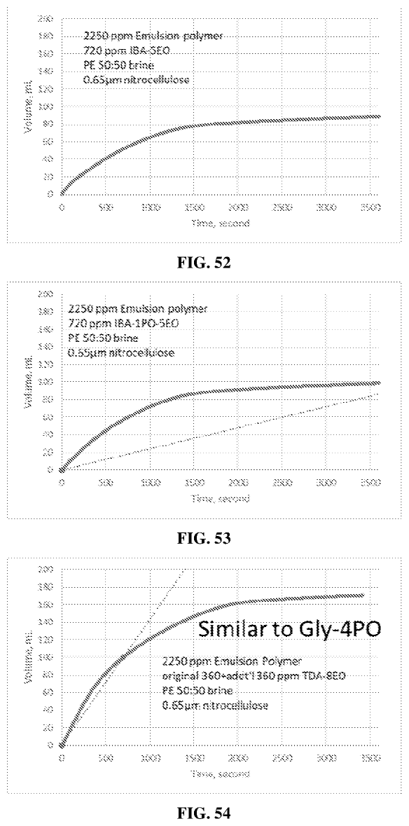

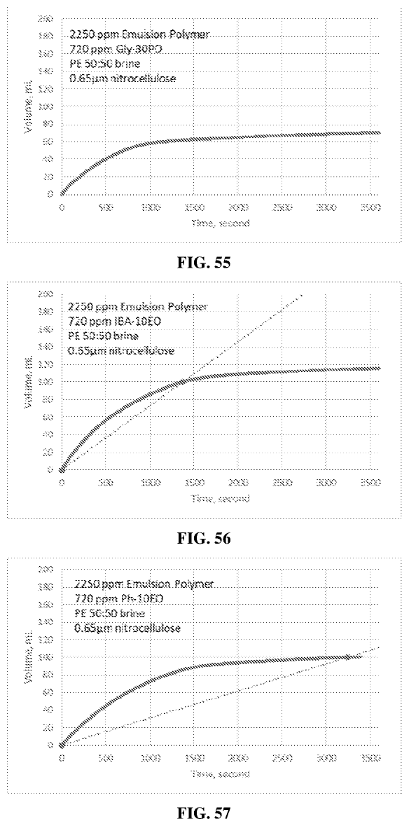

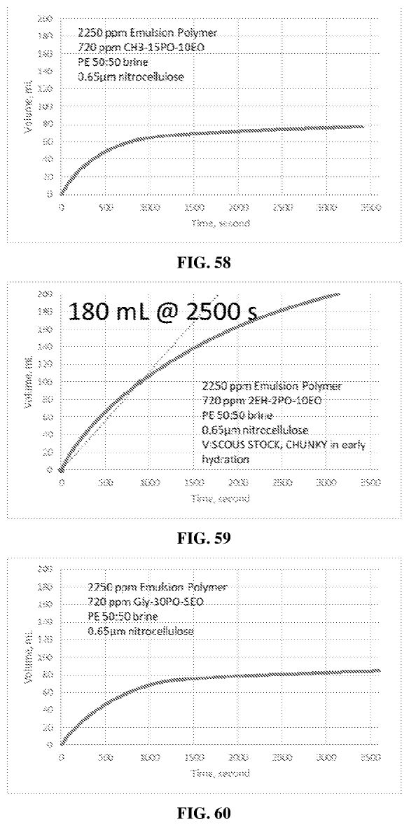

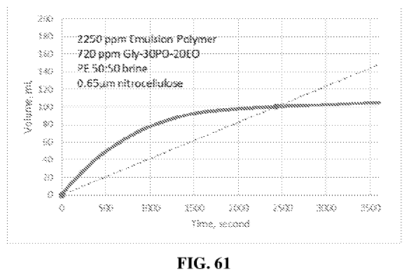

FIG. 22 is a plot showing the phase behavior over a range of salinity and oil concentration.

FIG. 23 shows the images of the phase behavior tubes using formulation 1: 1% MEA and 0.5% glycerine-30PO-35EO-OH, mixing Brine A and B at 21° C., where optimal salinity, e.g., salinity of lowest IFT Winsor type III, is identified by red arrows.

FIG. 24 show the images of phase behavior tubes using a formulation dilution with WF: 1% MEA and 0.5% glycerine-30PO-35EO-OH, mixing Brine A at 21° C., where optimal salinity, e.g., salinity of lowest IFT Winsor type III, is identified by red arrows.

FIG. 25 show the images of phase behavior tubes using a formulation dilution with WF: 1% MEA, 0.15% ABS, and 0.15% glycerine-30PO-10EO-OH, mixing Brine A at 21° C., where optimal salinity, e.g., salinity of lowest IFT Winsor type III, is identified by red arrows.

FIG. 26 shows the activity map constructed from the data generated using ASP formulation; 1% MEA, 0.15% ABS, and 0.15% glycerine-30PO-35EO-OH with Brine A at 21° C.

FIG. 27 shows the images of the phase behavior tubes using ASP formulation; 1% MEA, 0.15% ABS, and 0.15% glycerine-30PO-35EO-OH with Brine A at 21° C., where optimal salinity, e.g., salinity of lowest IFT Winsor type III, is identified by red arrows.

FIG. 28 shows the images of phase behavior tubes using a formulation dilution with WF: 1% MEA, 0.15% ABS, and 0.15% glycerine-30PO-10EO-OH, mixing Brine A at 21° C., where optimal salinity, e.g., salinity of lowest IFT Winsor type III, is identified by red arrows.

FIG. 29A-29B shows the images of phase behavior tubes using a formulation dilution with PD: 1% MEA, 0.15% ABS, and 0.15% glycerine-30PO-10EO-OH, mixing Brine A at 21° C., where optimal salinity, e.g., salinity of lowest IFT Winsor type III, is identified by red arrows. FIG. 29A shows PD: soft Brine B (2.4k ppm). FIG. 29B shows PD: mix of brine A+B (10k ppm).

FIG. 30 shows a graph of pressure drop during coreflood.

FIG. 31 shows a graph of volume versus time for formulation comprising emulsion polymer, and PE 50:50 brine, with filtration ratio of 1.11 with 1 μm polycarbonate filter.

FIG. 32 shows a graph of volume versus time for formulation comprising emulsion polymer, TDA-8PO-1EO-SO4, and PE 50:50 brine, with filtration ratio of 2.83 with 1 μm polycarbonate filter.

FIG. 33 shows a graph of volume versus time for formulation comprising emulsion polymer, Gly-4PO, and PE 50:50 brine, with filtration ratio of 1.06 with 1 μm polycarbonate filter.

FIG. 34 shows a graph of volume versus time for formulation comprising emulsion polymer, TMP-12PO, and PE 50:50 brine, with filtration ratio of 1.26 with 1 μm polycarbonate filter.

FIG. 35 shows a graph of volume versus time for formulation comprising emulsion polymer, IBA-1EO, and PE 50:50 brine, with filtration ratio of 1.06 with 1 μm polycarbonate filter.

FIG. 36 shows a graph of volume versus time for formulation comprising emulsion polymer, IBA-5EO, and PE 50:50 brine, with filtration ratio of 1.10 with 1 μm polycarbonate filter.

FIG. 37 shows a graph of volume versus time for formulation comprising emulsion polymer, IBA-1PO-5EO, and PE 50:50 brine, with filtration ratio of 1.22 with 1 μm polycarbonate filter.

FIG. 38 shows a graph of volume versus time for formulation comprising emulsion polymer, additional TDA-8EO, and PE 50:50 brine, with filtration ratio of 1.11 with 1 μm polycarbonate filter.

FIG. 39 shows a graph of volume versus time for formulation comprising emulsion polymer, Gly-30PO, and PE 50:50 brine, with filtration ratio of 1.41 with 1 μm polycarbonate filter.

FIG. 40 shows a graph of volume versus time for formulation comprising emulsion polymer, IBA-10EO, and PE 50:50 brine, with filtration ratio of 1.21 with 1 μm polycarbonate filter.

FIG. 41 shows a graph of volume versus time for formulation comprising emulsion polymer, Ph-10EO, and PE 50:50 brine, with filtration ratio of 1.18 with 1 μm polycarbonate filter.

FIG. 42 shows a graph of volume versus time for formulation comprising emulsion polymer, CH₃-15PO-10EO, and PE 50:50 brine, with filtration ratio of 1.72 with 1 μm polycarbonate filter.

FIG. 43 shows a graph of volume versus time for formulation comprising emulsion polymer, 2EH-2PO-10EO, and PE 50:50 brine, with filtration ratio of 1.03 with 1 μm polycarbonate filter.

FIG. 44 shows a graph of volume versus time for formulation comprising emulsion polymer, Gly-30PO-5EO, and PE 50:50 brine, with filtration ratio of 1.36 with 1 μm polycarbonate filter.

FIG. 45 shows a graph of volume versus time for formulation comprising emulsion polymer, Gly-30PO-20EO, and PE 50:50 brine, with filtration ratio of 1.92 with 1 μm polycarbonate filter.

FIG. 46 shows a graph of volume versus time for formulation comprising emulsion polymer, and PE 50:50 brine, with 0.65 μm nitrocellulose.

FIG. 47 shows a graph of volume versus time for formulation comprising emulsion polymer, TDA-8PO-1EO-SO₄, and PE 50:50 brine, with 0.65 μm nitrocellulose.

FIG. 48 shows a graph of volume versus time for formulation comprising emulsion polymer, Gly-4PO, and PE 50:50 brine, with 0.65 μm nitrocellulose.

FIG. 49 shows a graph of volume versus time for formulation comprising emulsion polymer, Gly-60PO-20EO, and PE 50:50 brine, with 0.65 μm nitrocellulose.

FIG. 50 shows a graph of volume versus time for formulation comprising emulsion polymer, TMP-12PO, and PE 50:50 brine, with 0.65 μm nitrocellulose.

FIG. 51 shows a graph of volume versus time for formulation comprising emulsion polymer, IBA-1EO, and PE 50:50 brine, with 0.65 μm nitrocellulose.

FIG. 52 shows a graph of volume versus time for formulation comprising emulsion polymer, IBA-5EO, and PE 50:50 brine, with 0.65 μm nitrocellulose.

FIG. 53 shows a graph of volume versus time for formulation comprising emulsion polymer, IBA-1PO-5EO, and PE 50:50 brine, with 0.65 μm nitrocellulose.

FIG. 54 shows a graph of volume versus time for formulation comprising emulsion polymer, additional TDA-8EO, and PE 50:50 brine, with 0.65 μm nitrocellulose.

FIG. 55 shows a graph of volume versus time for formulation comprising emulsion polymer, Gly-30PO, and PE 50:50 brine, with 0.65 μm nitrocellulose.

FIG. 56 shows a graph of volume versus time for formulation comprising emulsion polymer, IBA-10EO, and PE 50:50 brine, with 0.65 μm nitrocellulose.

FIG. 57 shows a graph of volume versus time for formulation comprising emulsion polymer, Ph-10EO, and PE 50:50 brine, with 0.65 μm nitrocellulose.

FIG. 58 shows a graph of volume versus time for formulation comprising emulsion polymer, CH3-15PO-10EO, and PE 50:50 brine, with 0.65 μm nitrocellulose.

FIG. 59 shows a graph of volume versus time for formulation comprising emulsion polymer, 2EH-2PO-10EO, and PE 50:50 brine, with 0.65 μm nitrocellulose.

FIG. 60 shows a graph of volume versus time for formulation comprising emulsion polymer, Gly-30PO-5EO, and PE 50:50 brine, with 0.65 μm nitrocellulose.

FIG. 61 shows a graph of volume versus time for formulation comprising emulsion polymer, Gly-30PO-20EO, and PE 50:50 brine, with 0.65 μm nitrocellulose.

Like reference symbols in the various drawings indicate like elements.

### DETAILED DESCRIPTION

To facilitate understanding of the disclosure set forth herein, a number of terms are defined below. Unless defined otherwise, all technical and scientific terms used herein generally have the same meaning as commonly understood by one of ordinary skill in the art to which this disclosure belongs. Publications cited herein and the materials for which they are cited are specifically incorporated by reference.

### General Definitions

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Accordingly, these terms are intended to not only cover the recited element(s) or step(s), but may also include other elements or steps not expressly recited. Furthermore, as used herein, the use of the terms “a”, “an”, and “the” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

The use of the term “about” applies to all numeric values, whether or not explicitly indicated. This term generally refers to a range of numbers that one of ordinary skill in the art would consider as a reasonable amount of deviation to the recited numeric values (i.e., having the equivalent function or result). For example, this term can be construed as including a deviation of ±10 percent of the given numeric value provided such a deviation does not alter the end function or result of the value. Therefore, a value of about 1% can be construed to be a range from 0.9% to 1.1%. Furthermore, a range may be construed to include the start and the end of the range. For example, a range of 10% to 20% (i.e., range of 10%-20%) can includes 10% and also includes 20%, and includes percentages in between 10% and 20%, unless explicitly stated otherwise herein.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

The term “contacting” as used herein, refers to materials or compounds being sufficiently close in proximity to react or interact. For example, in methods of contacting an unrefined petroleum material, a hydrocarbon material bearing formation, and/or a well bore, the term “contacting” can include placing a compound (e.g., a surfactant) or an aqueous composition (e.g., chemical, surfactant or polymer) within a hydrocarbon material-bearing formation using any suitable manner known in the art (e.g., pumping, injecting, pouring, releasing, displacing, spotting or circulating the chemical into a well, well bore or hydrocarbon bearing formation).

The terms “unrefined petroleum” and “crude oil” are used interchangeably and in keeping with the plain ordinary usage of those terms. “Unrefined petroleum” and “crude oil” may be found in a variety of petroleum reservoirs (also referred to herein as a “reservoir,” “oil field deposit” “deposit” and the like) and in a variety of forms including oleaginous materials, oil shales (i.e., organic-rich fine-grained sedimentary rock), tar sands, light oil deposits, heavy oil deposits, and the like. “Crude oils” or “unrefined petroleums” generally refer to a mixture of naturally occurring hydrocarbons that may be refined into diesel, gasoline, heating oil, jet fuel, kerosene, and other products called fuels or petrochemicals. Crude oils or unrefined petroleums are named according to their contents and origins, and are classified according to their per unit weight (specific gravity). Heavier crudes generally yield more heat upon burning, but have lower gravity as defined by the American Petroleum Institute (API) (i.e., API gravity) and market price in comparison to light (or sweet) crude oils. Crude oil may also be characterized by its Equivalent Alkane Carbon Number (EACN). The term “API gravity” refers to the measure of how heavy or light a petroleum liquid is compared to water. If an oil's API gravity is greater than 10, it is lighter and floats on water, whereas if it is less than 10, it is heavier and sinks. API gravity is thus an inverse measure of the relative density of a petroleum liquid and the density of water. API gravity may also be used to compare the relative densities of petroleum liquids. For example, if one petroleum liquid floats on another and is therefore less dense, it has a greater API gravity.

Crude oils vary widely in appearance and viscosity from field to field. They range in color, odor, and in the properties they contain. While all crude oils are mostly hydrocarbons, the differences in properties, especially the variation in molecular structure, determine whether a crude oil is more or less easy to produce, pipeline, and refine. The variations may even influence its suitability for certain products and the quality of those products. Crude oils are roughly classified into three groups, according to the nature of the hydrocarbons they contain. (i) Paraffin-based crude oils contain higher molecular weight paraffins, which are solid at room temperature, but little or no asphaltic (bituminous) matter. They can produce high-grade lubricating oils. (ii) Asphaltene based crude oils contain large proportions of asphaltic matter, and little or no paraffin. Some are predominantly naphthenes and so yield lubricating oils that are sensitive to temperature changes than the paraffin-based crudes. (iii) Mixed based crude oils contain both paraffin and naphthenes, as well as aromatic hydrocarbons. Most crude oils fit this latter category.

“Reactive” crude oil, as referred to herein, is crude oil containing natural organic acidic components (also referred to herein as unrefined petroleum acid) or their precursors such as esters or lactones. These reactive crude oils can generate soaps (carboxylates) when reacted with alkali. More terms used interchangeably for crude oil throughout this disclosure are hydrocarbon material or active petroleum material. An “oil bank” or “oil cut” as referred to herein, is the crude oil that does not contain the injected chemicals and is pushed by the injected fluid during an enhanced oil recovery process. A “nonactive oil,” as used herein, refers to an oil that is not substantially reactive or crude oil not containing significant amounts of natural organic acidic components or their precursors such as esters or lactones such that significant amounts of soaps are generated when reacted with alkali. A nonactive oil as referred to herein includes oils having an acid number of less than 0.5 mg KOH/g of oil.

“Unrefined petroleum acids” as referred to herein are carboxylic acids contained in active petroleum material (reactive crude oil). The unrefined petroleum acids contain C₁₁-C₂₀ alkyl chains, including napthenic acid mixtures. The recovery of such “reactive” oils may be performed using alkali (e.g., NaOH or Na₂CO₃) in a surfactant composition. The alkali reacts with the acid in the reactive oil to form soap in situ. These in situ generated soaps serve as a source of surfactants minimizing the levels of added surfactants, thus enabling efficient oil recovery from the reservoir.

The term “polymer” refers to a molecule having a structure that essentially includes the multiple repetitions of units derived, actually or conceptually, from molecules of low relative molecular mass. In some embodiments, the polymer is an oligomer.

The term “productivity” as applied to a petroleum or oil well refers to the capacity of a well to produce hydrocarbons (e.g., unrefined petroleum); that is, the ratio of the hydrocarbon flow rate to the pressure drop, where the pressure drop is the difference between the average reservoir pressure and the flowing bottom hole well pressure (i.e., flow per unit of driving force).

The term “oil solubilization ratio” is defined as the volume of oil solubilized divided by the volume of surfactant in microemulsion. All the surfactant is presumed to be in the microemulsion phase. The oil solubilization ratio is applied for Winsor type I and type III behavior. The volume of oil solubilized is found by reading the change between initial aqueous level and excess oil (top) interface level. The oil solubilization ratio is calculated as follows:

$${σ}_{o} = \frac{{V}_{o}}{{V}_{s}}$$

where σₒ is the oil solubilization ratio, Vₒ is the volume of oil solubilized, and Vs is the volume of surfactant.

The term “water solubilization ratio” is defined as the volume of water solubilized divided by the volume of surfactant in microemulsion. All the surfactant is presumed to be in the microemulsion phase. The water solubilization ratio is applied for Winsor type III and type II behavior. The volume of water solubilized is found by reading the change between initial aqueous level and excess water (bottom) interface level. The water solubilization parameter is calculated as follows:

$${σ}_{w} = \frac{{V}_{w}}{{V}_{s}}$$

where σ_{w} is the water solubilization ratio, V_{w} is the volume of oil solubilized, and Vs is the volume of surfactant.

The optimum solubilization ratio occurs where the oil and water solubilization ratios are equal. The coarse nature of phase behavior screening often does not include a data point at optimum, so the solubilization ratio curves are drawn for the oil and water solubilization ratio data and the intersection of these two curves is defined as the optimum. The following is true for the optimum solubilization ratio:

$${σ}_{o} = {σ}_{w} = {σ}^{s}$$

where σ* is the optimum solubilization ratio.

The term “solubility” or “solubilization” in general refers to the property of a solute, which can be a solid, liquid or gas, to dissolve in a solid, liquid or gaseous solvent thereby forming a homogenous solution of the solute in the solvent. Solubility occurs under dynamic equilibrium, which means that solubility results from the simultaneous and opposing processes of dissolution and phase joining (e.g., precipitation of solids). The solubility equilibrium occurs when the two processes proceed at a constant rate. The solubility of a given solute in a given solvent typically depends on temperature. For many solids dissolved in liquid water, the solubility increases with temperature. In liquid water at high temperatures, the solubility of ionic solutes tends to decrease due to the change of properties and structure of liquid water. In more particular, solubility and solubilization as referred to herein is the property of oil to dissolve in water and vice versa.

“Viscosity” refers to a fluid's internal resistance to flow or being deformed by shear or tensile stress. In other words, viscosity may be defined as thickness or internal friction of a liquid. Thus, water is “thin”, having a lower viscosity, while oil is “thick”, having a higher viscosity. More generally, the less viscous a fluid is, the greater its ease of fluidity.

The term “salinity” as used herein, refers to concentration of salt dissolved in an aqueous phases. Examples for such salts are without limitation, sodium chloride, magnesium and calcium sulfates, and bicarbonates. In more particular, the term salinity as it pertains to the present invention refers to the concentration of salts in brine and surfactant solutions.

The term “aqueous solution or aqueous formulation” refers to a solution in which the solvent is water. The term “emulsion, emulsion solution or emulsion formulation” refers to a mixture of two or more liquids which are normally immiscible. A non-limiting example for an emulsion is a mixture of oil and water.

The term “co-solvent,” as used herein, refers to a compound having the ability to increase the solubility of a solute (e.g., a surfactant as disclosed herein) in the presence of an unrefined petroleum acid. In some embodiments, the co-solvents provided herein have a hydrophobic portion (alkyl or aryl chain), a hydrophilic portion (e.g., an alcohol) and an alkoxy portion.

The term “interfacial tension” or “IFT” as used herein refers to the surface tension between test oil and water of different salinities containing a surfactant formulation at different concentrations. Typically, interfacial tensions are measured using a spinning drop tensiometer or calculated from phase behavior experiments.

The term “contacting” as used herein, refers to materials or compounds being sufficiently close in proximity to react or interact. For example, in methods of contacting an unrefined petroleum material, a hydrocarbon-bearing formation, and/or a wellbore, the term “contacting” can include placing a compound (e.g., a surfactant) or an aqueous composition (e.g., chemical, surfactant or polymer) within a hydrocarbon-bearing formation using any suitable manner known in the art (e.g., pumping, injecting, pouring, releasing, displacing, spotting or circulating the chemical into a well, wellbore or hydrocarbon-bearing formation).

### Chemical Definitions

Terms used herein will have their customary meaning in the art unless specified otherwise. The organic moieties mentioned when defining variable positions within the general formulae described herein (e.g., the term “halogen”) are collective terms for the individual substituents encompassed by the organic moiety. Ph in Formula I refers to a phenyl group.

The prefix Cₙ-Cₘ preceding a group or moiety indicates, in each case, the possible number of carbon atoms in the group or moiety that follows.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, heteroatoms present in a compound or moiety, such as nitrogen, can have hydrogen substituents and/or any permissible substituents of organic compounds described herein which satisfy the valency of the heteroatom. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound (e.g., a compound that does not spontaneously undergo transformation such as by rearrangement, cyclization, elimination, etc.

The term “optionally substituted,” as used herein, means that substitution with an additional group is optional and therefore it is possible for the designated atom to be unsubstituted. Thus, by use of the term “optionally substituted” the disclosure includes examples where the group is substituted and examples where it is not.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

As used herein, the term “alkyl” refers to saturated, straight-chained or branched saturated hydrocarbon moieties. Unless otherwise specified, C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, and 1-ethyl-2-methyl-propyl. Alkyl substituents may be unsubstituted or substituted with one or more chemical moieties. The alkyl group can be substituted with one or more groups including, but not limited to, hydroxy, halogen, acyl, alkyl, alkoxy, alkenyl, alkynyl, aryl, heteroaryl, acyl, aldehyde, amino, carboxylic acid, ester, ether, ketone, nitro, silyl, sulfo-oxo, sulfonyl, sulfone, sulfoxide, thiosulfonate (e.g., —SSO₂Ra), or thiol, as described below, provided that the substituents are sterically compatible and the rules of chemical bonding and strain energy are satisfied. The alkyl group can also include one or more heteroatoms (e.g., from one to three heteroatoms) incorporated within the hydrocarbon moiety. Examples of heteroatoms include, but are not limited to, nitrogen, oxygen, sulfur, and phosphorus.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. The term “alkylthiol” specifically refers to an alkyl group that is substituted with one or more thiol groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight-chained, or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyl refers to a group with the structure-CH═CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, thiosulfonate (e.g., —SSO₂Ra), or thiol, as described below, provided that the substituents are sterically compatible and the rules of chemical bonding and strain energy are satisfied.

As used herein, the term “alkynyl” represents straight-chained or branched hydrocarbon moieties containing a triple bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, thiosulfonate (e.g., —SSO₂Ra), or thiol, as described below.

As used herein, the term “aryl,” as well as derivative terms such as aryloxy, refers to groups that include a monovalent aromatic carbocyclic group of from 3 to 20 carbon atoms. Aryl groups can include a single ring or multiple condensed rings. In some embodiments, aryl groups include C₆-C₁₀ aryl groups. Examples of aryl groups include, but are not limited to, phenyl, biphenyl, naphthyl, tetrahydronaphthyl, phenylcyclopropyl, and indanyl. In some embodiments, the aryl group can be a phenyl, indanyl or naphthyl group. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, cycloalkyl, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl has 5-10 ring atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, and 7-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by oxo or sulfido (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl has 4-10, 4-7 or 4-6 ring atoms with 1 or 2 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The term “acyl” as used herein is represented by the formula —C(O)Z¹ where Z¹ can be a hydrogen, hydroxyl, alkoxy, alkyl, halogenated alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a short hand notation for C═O.

As used herein, the term “alkoxy” refers to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₅, C₁-C₁₆, C₁-C₁₄, C₁-C₁₂, C₁-C₁₀, C₁-C₈, C₁-C₆, C₁-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-penoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “aldehyde” as used herein is represented by the formula —C(O)H.

The terms “amine” or “amino” as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above. “Amido” is —C(O)NZ¹Z².

The term “carboxylic acid” as used herein is represented by the formula —C(O)OH. A “carboxylate” or “carboxyl” group as used herein is represented by the formula —C(O)O⁻.

The term “ester” as used herein is represented by the formula —OC(O)Z¹ or —C(O)OZ¹, where Z¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” or “halogen” or “halo” as used herein refers to fluorine, chlorine, bromine, and iodine.

The term “hydroxyl” as used herein is represented by the formula —OH.

The term “nitro” as used herein is represented by the formula —NO₂.

The term “silyl” as used herein is represented by the formula —S¹Z¹Z²Z³, where Z¹, Z², and Z³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula —S(O)₂Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “thiol” as used herein is represented by the formula —SH.

The term “thio” as used herein is represented by the formula —S—.

As used herein, Me refers to a methyl group; OMe refers to a methoxy group; and i-Pr refers to an isopropyl group.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

The term “substituted” refers to a molecule wherein at least one hydrogen atom is replaced with a substituent. When substituted, one or more of the groups are “substituents.” The molecule can be multiply substituted. In the case of an oxo substituent (“═O”), two hydrogen atoms are replaced. Example substituents within this context can include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, —NRaSO₂Rb, —C(═O)Ra, —C(═O)ORa, —C(═O)NRaRb, —OC(═O)NRaRb, —ORa, —SRa, —SORa, —S(═O)₂Ra, —OS(═O)₂Ra and —S(═O)₂ORa. Ra and Rb in this context can be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

Reference will now be made in detail to specific aspects of the disclosed materials, compounds, compositions, articles, and methods, examples of which are illustrated in the accompanying Examples and Figures.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are both chemically related may be substituted for the agents described herein while the same or similar results would be achieved. All such similar substitutes and modifications apparent to those skilled in the art are deemed to be within the spirit, scope and concept of the disclosure as defined by the appended claims.

### Compounds

Provided herein are polyol alkoxylate surfactants. The polyol alkoxylate surfactant can be a compound defined by Formula I below

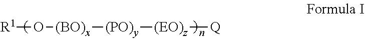

wherein

BO represents —CH₂—CH(ethyl)-O— or —CH₃CH(O—)CH₃;

PO represents —CH₂—CH(methyl)-O—;

EO represents —CH₂—CH₂—O—;

R¹ represents a C₁-C₆ alkyl group or a cyclic structure derived from alkoxylation of an alkyl monoglucoside, an alkyl polyglucoside, a monosaccharide, a disaccharide, or a polysaccharide;

n is an integer from 2 to 6;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation;

x is an integer from 0 to 15;

y is an integer from 11 to 60; and

z is an integer from 0 to 100.

In some embodiments, n can be an integer from 3 to 6, from 3 to 5, from 3 to 4, from 4 to 5, or from 4 to 6. In some embodiments, n can be an integer from 3 to 6. In some embodiments, n can be 3, 4, 5, or 6. In some embodiments, n is 3.

In some embodiments, R¹ can be a C₁-C₅ alkyl, C₁-C₄ alkyl, C₁-C₃ alkyl, C₁-C₂ alkyl, C₂-C₆ alkyl, C₂-C₅ alkyl, C₂-C₄ alkyl, C₂-C₃ alkyl, C₃-C₆ alkyl, C₃-C₅ alkyl, C₃-C₄ alkyl, C₄-C₅ alkyl, C₄-C₆ alkyl, or C₅-C₆ alkyl group. In some embodiments, R¹ can be a C₃-C₆ alkyl group.

In other embodiments, R¹ can comprise a monocyclic or polycyclic structure derived from alkoxylation of an alkyl monoglucoside, an alkyl polyglucoside, a monosaccharide, a disaccharide, or a polysaccharide.

In some embodiments, the compound of Formula I can be defined by Formula II below

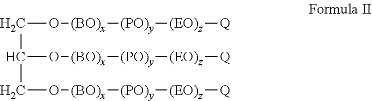

wherein

BO represents —CH₂—CH(ethyl)-O— or —CH₃CH(O—)CH₃;

PO represents —CH₂—CH(methyl)-O—;

EO represents —CH₂—CH₂—O—;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation;

x is an integer from 0 to 15;

y is an integer from 11 to 60; and

z is an integer from 0 to 100.

In some embodiments, the compound of Formula I can be defined by Formula III below

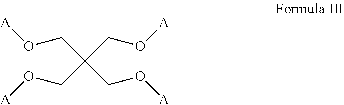

wherein

A represents —(BO)ₓ—(PO)_{y}-(EO)_{z}-Q

BO represents —CH₂—CH(ethyl)-O— or —CH₃CH(O—)CH₃;

PO represents —CH₂—CH(methyl)-O—;

EO represents —CH₂—CH₂—O—;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation;

x is an integer from 0 to 15;

y is an integer from 11 to 60; and

z is an integer from 0 to 100.

In some embodiments, the compound of Formula I can be defined by Formula III-a below

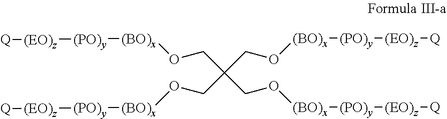

wherein

BO represents —CH₂—CH(ethyl)-O— or —CH₃CH(O—)CH₃;

PO represents —CH₂—CH(methyl)-O—;

EO represents —CH₂—CH₂—O—;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation;

x is an integer from 0 to 15;

y is an integer from 11 to 60; and

z is an integer from 0 to 100.

In some embodiments, the compound of Formula I can be defined by Formula IV below

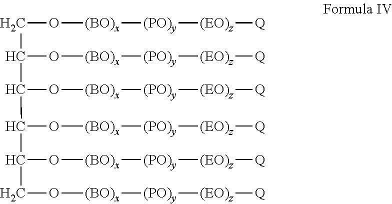

wherein

BO represents —CH₂—CH(ethyl)-O— or —CH₃CH(O—)CH₃;

PO represents —CH₂—CH(methyl)-O—;

EO represents —CH₂—CH₂—O—;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation;

x is an integer from 0 to 15;

y is an integer from 11 to 60; and

z is an integer from 0 to 100.

In some embodiments, the compound of Formula I can be defined by Formula V below

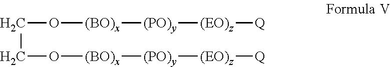

wherein

BO represents —CH₂—CH(ethyl)-O— or —CH₃CH(O—)CH₃;

PO represents —CH₂—CH(methyl)-O—;

EO represents —CH₂—CH₂—O—;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation;

x is an integer from 0 to 15;

y is an integer from 11 to 60; and

z is an integer from 0 to 100.

In some embodiments, the compound of Formula I can be defined of Formula VI below

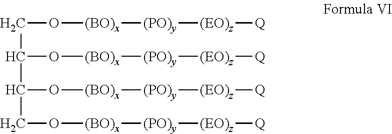

wherein

BO represents —CH₂—CH(ethyl)-O— or —CH₃CH(O—)CH₃;

PO represents —CH₂—CH(methyl)-O—;

EO represents —CH₂—CH₂—O—;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation;

x is an integer from 0 to 15;

y is an integer from 11 to 60; and

z is an integer from 0 to 100.

In some embodiments, the compound of Formula I can be defined of Formula VII below

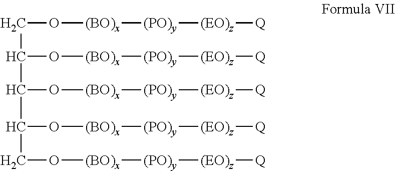

wherein

BO represents —CH₂—CH(ethyl)-O— or —CH₃CH(O—)CH₃;

PO represents —CH₂—CH(methyl)-O—;

EO represents —CH₂—CH₂—O—;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation;

x is an integer from 0 to 15;

y is an integer from 11 to 60; and

z is an integer from 0 to 100.

In some embodiments, the compound of Formula I can be defined by Formula VIII below

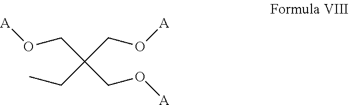

wherein

A represents —(BO)ₓ—(PO)_{y}-(EO)_{z}-Q

BO represents —CH₂—CH(ethyl)-O— or —CH₃CH(O—)CH₃;

PO represents —CH₂—CH(methyl)-O—;

EO represents —CH₂—CH₂—O—;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation;

x is an integer from 0 to 15;

y is an integer from 11 to 60; and

z is an integer from 0 to 100.

In some embodiments, the compound of Formula I can be defined by Formula IV-a below

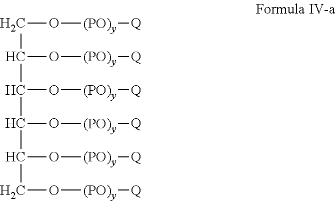

wherein

PO represents —CH₂—CH(methyl)-O—;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation; and

y is an integer from 11 to 60.

In some embodiments, the compound of Formula I can be defined by Formula V-a below

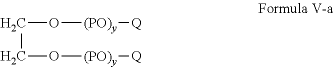

wherein

PO represents —CH₂—CH(methyl)-O—;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation; and

y is an integer from 11 to 60.

In some embodiments, the compound of Formula I can be defined by Formula II-a below

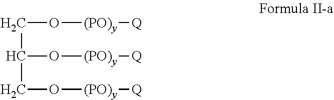

wherein

PO represents —CH₂—CH(methyl)-O—;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation; and

y is an integer from 11 to 60.

In some embodiments, the compound of Formula I can be defined by Formula VI-a below

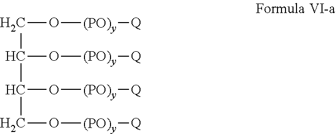

wherein

PO represents —CH₂—CH(methyl)-O—;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation; and

y is an integer from 11 to 60.

In some embodiments, the compound of Formula I can be defined by Formula VII-a below

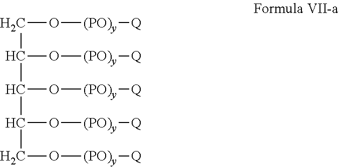

wherein

PO represents —CH₂—CH(methyl)-O—;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation; and

y is an integer from 11 to 60.

In some embodiments, the compound of Formula I can be defined by Formula VIII-a below

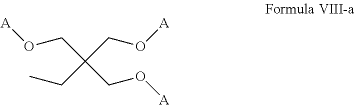

wherein

A represents —(PO)_{y}-Q

PO represents —CH₂—CH(methyl)-O—;

R¹ represents a C₁-C₆ alkyl group;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation;

y is an integer from 11 to 60.

In some embodiments, the compound of Formula I can be defined by Formula II-b below

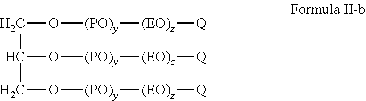

wherein

PO represents —CH₂—CH(methyl)-O—;

EO represents —CH₂—CH₂—O—;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation;

y is an integer from 11 to 60; and

z is an integer from 0 to 100.

In some embodiments, the compound of Formula I can be defined by Formula III-b below

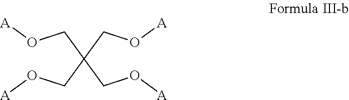

wherein

A represents —(PO)_{y}-(EO)_{z}-Q

PO represents —CH₂—CH(methyl)-O—;

EO represents —CH₂—CH₂—O—;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation;

y is an integer from 11 to 60; and

z is an integer from 0 to 100.

In some embodiments, the compound of Formula I can be defined by Formula III-c below

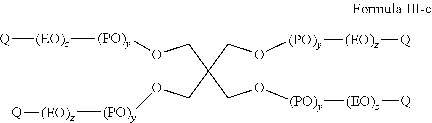

wherein

PO represents —CH₂—CH(methyl)-O—;

EO represents —CH₂—CH₂—O—;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation;

y is an integer from 11 to 60; and

z is an integer from 0 to 100.

In some embodiments, the compound of Formula I can be defined by Formula IV-b below

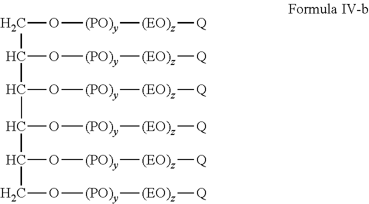

wherein

PO represents —CH₂—CH(methyl)-O—;

EO represents —CH₂—CH₂—O—;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation;

y is an integer from 11 to 60; and

z is an integer from 0 to 100.

In some embodiments, the compound of Formula I can be defined by Formula V-b below

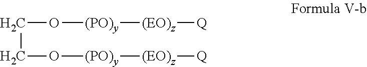

wherein

PO represents —CH₂—CH(methyl)-O—;

EO represents —CH₂—CH₂—O—;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation;

y is an integer from 11 to 60; and

z is an integer from 0 to 100.

In some embodiments, the compound of Formula I can be defined of Formula VI-b below

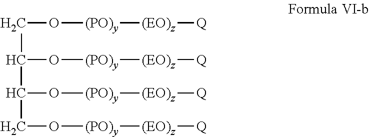

wherein

PO represents —CH₂—CH(methyl)-O—;

EO represents —CH₂—CH₂—O—;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation;

y is an integer from 11 to 60; and

z is an integer from 0 to 100.

In some embodiments, the compound of Formula I can be defined of Formula VII-b below

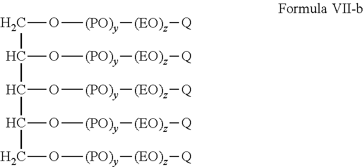

wherein

PO represents —CH₂—CH(methyl)-O—;

EO represents —CH₂—CH₂—O—;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation;

y is an integer from 11 to 60; and

z is an integer from 0 to 100.

In some embodiments, the compound of Formula I can be defined by Formula VIII-b below

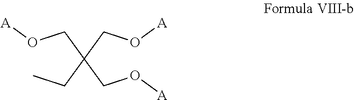

wherein

A represents —(PO)_{y}-(EO)_{z}-Q

PO represents —CH₂—CH(methyl)-O—;

EO represents —CH₂—CH₂—O—;

R¹ represents a C₁-C₆ alkyl group;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation;

y is an integer from 11 to 60; and

z is an integer from 0 to 100.

In some embodiments, the compound of Formula I can be defined by Formula II-c below

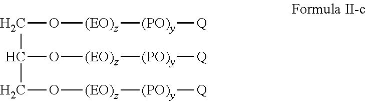

wherein

PO represents —CH₂—CH(methyl)-O—;

EO represents —CH₂—CH₂—O—;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation;

y is an integer from 11 to 60; and

z is an integer from 0 to 100.

In some embodiments, the compound of Formula I can be defined by Formula III-d below

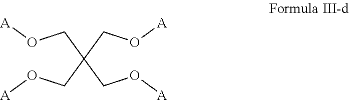

wherein

A represents -(EO)_{z}—(PO)_{y}-Q

PO represents —CH₂—CH(methyl)-O—;

EO represents —CH₂—CH₂—O—;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation;

y is an integer from 11 to 60; and

z is an integer from 0 to 100.

In some embodiments, the compound of Formula I can be defined by Formula III-e below

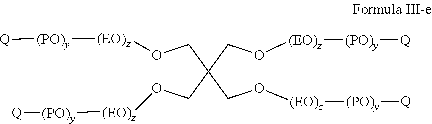

wherein

PO represents —CH₂—CH(methyl)-O—;

EO represents —CH₂—CH₂—O—;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation;

y is an integer from 11 to 60; and

z is an integer from 0 to 100.

In some embodiments, the compound of Formula I can be defined by Formula IV-c below

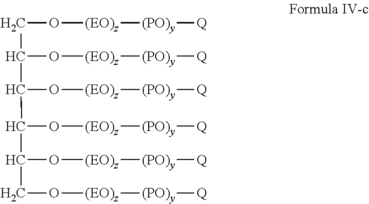

wherein

PO represents —CH₂—CH(methyl)-O—;

EO represents —CH₂—CH₂—O—;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation;

y is an integer from 11 to 60; and

z is an integer from 0 to 100.

In some embodiments, the compound of Formula I can be defined by Formula V-c below

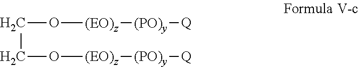

wherein

PO represents —CH₂—CH(methyl)-O—;

EO represents —CH₂—CH₂—O—;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation;

y is an integer from 11 to 60; and

z is an integer from 0 to 100.

In some embodiments, the compound of Formula I can be defined of Formula VI-c below

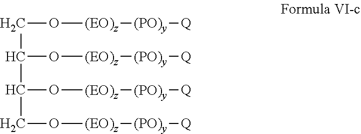

wherein

PO represents —CH₂—CH(methyl)-O—;

EO represents —CH₂—CH₂—O—;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation;

y is an integer from 11 to 60; and

z is an integer from 0 to 100.

In some embodiments, the compound of Formula I can be defined of Formula VII-c below

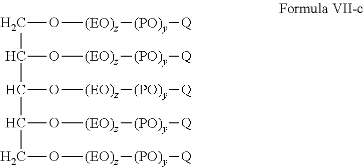

wherein

PO represents —CH₂—CH(methyl)-O—;

EO represents —CH₂—CH₂—O—;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation;

y is an integer from 11 to 60; and

z is an integer from 0 to 100.

In some embodiments, the compound of Formula I can be defined by Formula VIII-c below

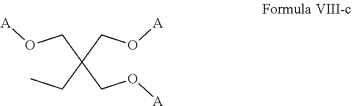

wherein

A represents -(EO)_{z}—(PO)_{y}-Q

PO represents —CH₂—CH(methyl)-O—;

EO represents —CH₂—CH₂—O—;

R¹ represents a C₁-C₆ alkyl group;

Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation;

y is an integer from 11 to 60; and

z is an integer from 0 to 100.

In some embodiments, Q can be —SO₃M⁺, —SO₃H, —CH₂C(O)O⁻M⁺, or —CH₂C(O)OH; and M⁺ is a cation. In some embodiments, Q can be —SO₃M⁺; and M⁺ is a cation. In some embodiments, Q can be —CH₂C(O)O⁻M⁺; and M⁺ is a cation. In some embodiments, Q can be —SO₃H. In some embodiments, Q can be —CH₂C(O)OH. In some embodiments, Q can be CH₂CH(OH)CH₂—SO₃M⁺, and M⁺ is a cation. In some embodiments, Q can be CH₂CH(OH)CH₂—SO₃H.

In some embodiments, y can be an integer from 11 to 60, (e.g., from 11 to 20, from 11 to 25, from 11 to 30, from 11 to 35, from 11 to 40, from 11 to 45, from 11 to 50, from 11 to 55, from 15 to 60, from 15 to 25, from 15 to 30, from 15 to 35, from 15 to 40, from 15 to 50, from 15 to 60, from 20 to 30, from 20 to 35, from 20 to 40, from 20 to 50, from 20 to 60, from 25 to 30, from 25 to 40, from 25 to 50, from 25 to 60, from 30 to 40, from 30 to 45, from 30 to 50, from 30 to 60, from 40 to 50, from 40 to 60, or from 50 to 40). In some embodiments, y can be 11. In some embodiments, y can be 15. In some embodiments, y can be 20. In some embodiments, y can be 25.

In some embodiments, x can be an integer from 0 to 15 (e.g., from 0 to 11, from 0 to 12, from 0 to 13, from 0 to 14, from 0 to 10, from 0 to 9, from 0 to 8, from 0 to 7, from 0 to 6, from 0 to 5, from 0 to 4, from 0 to 3, from 0 to 2, from 0 to 1, from 1 to 15, from 2 to 15, from 3 to 15, from 4 to 15, from 5 to 15, from 6 to 15, from 7 to 15, from 8 to 15, from 9 to 15, from 10 to 15, from 11 to 15, from 12 to 15, from 13 to 15, from 14 to 15, from 1 to 12, from 2 to 12, from 3 to 12, from 4 to 12, from 5 to 12, from 6 to 12, from 7 to 12, from 8 to 12, from 9 to 12, from 10 to 12, from 11 to 12, from 1 to 10, from 2 to 10, from 3 to 10, from 4 to 10, from 5 to 10, from 6 to 10, from 7 to 10, from 8 to 10, from 9 to 10, from 1 to 9, from 2 to 9, from 3 to 9, from 4 to 9, from 5 to 9, from 6 to 9, from 7 to 9, from 8 to 9, from 1 to 8, from 2 to 8, from 3 to 8, from 4 to 8, from 5 to 8, from 6 to 8, from 7 to 8, from 1 to 7, from 2 to 7, from 3 to 7, from 4 to 7, from 5 to 7, from 6 to 7, from 1 to 6, from 2 to 6, from 3 to 6, from 4 to 6, from 5 to 6, from 1 to 5, from 2 to 5, from 3 to 5, from 4 to 5, from 1 to 4, from 2 to 4, from 3 to 4, from 1 to 3, from 2 to 3, or from 1 to 2). In some embodiments, x can be 0, 1, 2, 3, 4, 5, 6, 7, 8, 9, 10, 11, 12, 13, 14, or 15. In some embodiments, x can be 0.

In some embodiments, z can be an integer from 0 to 100 (e.g. from 0 to 90, from 1 to 90, from 5 to 90, from 10 to 90, from 15 to 90, from 20 to 90, from 25 to 90, from 30 to 90, from 40 to 90, from 50 to 90, from 60 to 90, from 70 to 90, from 80 to 90, from 0 to 80, from 1 to 80, from 5 to 80, from 10 to 80, from 15 to 80, from 20 to 80, from 25 to 80, from 30 to 80, from 40 to 80, from 50 to 80, from 60 to 80, from 70 to 80, from 0 to 70, from 1 to 70, from 5 to 70, from 10 to 70, from 15 to 70, from 20 to 70, from 25 to 70, from 30 to 70, from 40 to 70, from 50 to 70, from 0 to 50, from 1 to 50, from 5 to 50, from 10 to 50, from 15 to 50, from 20 to 50, from 25 to 50, from 30 to 50, from 40 to 50, from 0 to 40, from 1 to 40, from 5 to 40, from 10 to 40, from 15 to 40, from 20 to 40, from 25 to 40, from 30 to 40, from 0 to 35, from 1 to 35, from 5 to 35, from 10 to 35, from 15 to 35, from 20 to 35, from 25 to 35, from 0 to 30, from 1 to 30, from 5 to 30, from 10 to 30, from 15 to 30, from 20 to 30, from 25 to 30, from 0 to 25, from 1 to 25, from 5 to 25, from 10 to 25, from 15 to 25, from 20 to 25, from 0 to 20, from 1 to 20, from 5 to 20, from 10 to 20, from 15 to 20, from 0 to 15, from 1 to 15, from 5 to 15, from 10 to 15, from 0 to 10, from 1 to 10, from 5 to 10, from 0 to 5, from 1 to 5, from 2 to 5, from 3 to 5, from 4 to 5, from 0 to 4, from 1 to 4, from 2 to 4, from 3 to 4, from 0 to 3, from 1 to 3, from 2 to 3, from 0 to 2, from 1 to 2, from 0 to 1). In some embodiments, z can be 0, 5, 10, 15, 20, 25, 30, 35, 40, 45, 50, 55, 60, 65, 70, 75, 80, 85, 90, 95, or 100. In some embodiments, z can be 0.

In some embodiments, z is greater than 1. In some embodiments, z is greater than 5, greater than 10, greater than 15, greater than 20, greater than 25, greater than 30, greater than 35, greater than 40, greater than 45, greater than 50, greater than 55, greater than 60, greater than 65, greater than 70, greater than 75, greater than 80, greater than 85, greater than 90, or greater than 95. In some embodiments, when Q is hydrogen, z can be at least 6 (e.g., at least 8, at least 10, at least 15, at least 20, at least 30, at least 45, at least 50, at least 55, at least 60, at least 65, at least 70, at least 75, at least 80, at least 85, at least 90, or at least 95).

In some embodiments, the compound can be defined by Formula IX

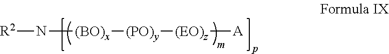

wherein

BO represents —CH₂—CH(ethyl)-O— or —CH₃CH(O—)CH₃;

PO represents —CH₂—CH(methyl)-O—;

EO represents —CH₂—CH₂—O—;

R² is absent, —((BO)ₓ—(PO)_{y}-(EO)_{z}-)ₘ-A, or —CH₂R³;

R³ is C₁-C₁₀ alkoxy, aryloxy, —C(O)O⁻M⁺, or —C(O)OH;

m is an integer from 1 to 3;

p is an integer from 2 to 3;

A is H, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;

M⁺, when present, is a cation;

x is an integer from 0 to 15;

y is an integer from 11 to 60; and

z is an integer from 0 to 100, wherein when R² is absent, p is 3.

In some embodiments, A can be H. In some embodiments, A can be —SO₃M⁺, —SO₃H, —CH₂C(O)O⁻M⁺, or —CH₂C(O)OH; and M⁺ is a cation. In some embodiments, A can be —SO₃M⁺; and M⁺ is a cation. In some embodiments, A can be —CH₂C(O)O⁻M⁺; and M⁺ is a cation. In some embodiments, A can be —SO₃H. In some embodiments, A can be —CH₂C(O)OH. In some embodiments, A can be CH₂CH(OH)CH₂—SO₃M⁺, and M⁺ is a cation. In some embodiments, A can be CH₂CH(OH)CH₂—SO₃H.

In some embodiments, y can be an integer from 11 to 60, (e.g., from 11 to 20, from 11 to 25, from 11 to 30, from 11 to 35, from 11 to 40, from 11 to 45, from 11 to 50, from 11 to 55, from 15 to 60, from 15 to 25, from 15 to 30, from 15 to 35, from 15 to 40, from 15 to 50, from 15 to 60, from 20 to 30, from 20 to 35, from 20 to 40, from 20 to 50, from 20 to 60, from 25 to 30, from 25 to 40, from 25 to 50, from 25 to 60, from 30 to 40, from 30 to 45, from 30 to 50, from 30 to 60, from 40 to 50, from 40 to 60, or from 50 to 40). In some embodiments, y can be 11. In some embodiments, y can be 15. In some embodiments, y can be 20. In some embodiments, y can be 25.

In some embodiments, x can be an integer from 0 to 15 (e.g., from 0 to 11, from 0 to 12, from 0 to 13, from 0 to 14, from 0 to 10, from 0 to 9, from 0 to 8, from 0 to 7, from 0 to 6, from 0 to 5, from 0 to 4, from 0 to 3, from 0 to 2, from 0 to 1, from 1 to 15, from 2 to 15, from 3 to 15, from 4 to 15, from 5 to 15, from 6 to 15, from 7 to 15, from 8 to 15, from 9 to 15, from 10 to 15, from 11 to 15, from 12 to 15, from 13 to 15, from 14 to 15, from 1 to 12, from 2 to 12, from 3 to 12, from 4 to 12, from 5 to 12, from 6 to 12, from 7 to 12, from 8 to 12, from 9 to 12, from 10 to 12, from 11 to 12, from 1 to 10, from 2 to 10, from 3 to 10, from 4 to 10, from 5 to 10, from 6 to 10, from 7 to 10, from 8 to 10, from 9 to 10, from 1 to 9, from 2 to 9, from 3 to 9, from 4 to 9, from 5 to 9, from 6 to 9, from 7 to 9, from 8 to 9, from 1 to 8, from 2 to 8, from 3 to 8, from 4 to 8, from 5 to 8, from 6 to 8, from 7 to 8, from 1 to 7, from 2 to 7, from 3 to 7, from 4 to 7, from 5 to 7, from 6 to 7, from 1 to 6, from 2 to 6, from 3 to 6, from 4 to 6, from 5 to 6, from 1 to 5, from 2 to 5, from 3 to 5, from 4 to 5, from 1 to 4, from 2 to 4, from 3 to 4, from 1 to 3, from 2 to 3, or from 1 to 2). In some embodiments, x can be 0, 1, 2, 3, 4, 5, 6, 7, 8, 9, 10, 11, 12, 13, 14, or 15. In some embodiments, x can be 0.

In some embodiments, z can be an integer from 0 to 100 (e.g. from 0 to 90, from 1 to 90, from 5 to 90, from 10 to 90, from 15 to 90, from 20 to 90, from 25 to 90, from 30 to 90, from 40 to 90, from 50 to 90, from 60 to 90, from 70 to 90, from 80 to 90, from 0 to 80, from 1 to 80, from 5 to 80, from 10 to 80, from 15 to 80, from 20 to 80, from 25 to 80, from 30 to 80, from 40 to 80, from 50 to 80, from 60 to 80, from 70 to 80, from 0 to 70, from 1 to 70, from 5 to 70, from 10 to 70, from 15 to 70, from 20 to 70, from 25 to 70, from 30 to 70, from 40 to 70, from 50 to 70, from 0 to 50, from 1 to 50, from 5 to 50, from 10 to 50, from 15 to 50, from 20 to 50, from 25 to 50, from 30 to 50, from 40 to 50, from 0 to 40, from 1 to 40, from 5 to 40, from 10 to 40, from 15 to 40, from 20 to 40, from 25 to 40, from 30 to 40, from 0 to 35, from 1 to 35, from 5 to 35, from 10 to 35, from 15 to 35, from 20 to 35, from 25 to 35, from 0 to 30, from 1 to 30, from 5 to 30, from 10 to 30, from 15 to 30, from 20 to 30, from 25 to 30, from 0 to 25, from 1 to 25, from 5 to 25, from 10 to 25, from 15 to 25, from 20 to 25, from 0 to 20, from 1 to 20, from 5 to 20, from 10 to 20, from 15 to 20, from 0 to 15, from 1 to 15, from 5 to 15, from 10 to 15, from 0 to 10, from 1 to 10, from 5 to 10, from 0 to 5, from 1 to 5, from 2 to 5, from 3 to 5, from 4 to 5, from 0 to 4, from 1 to 4, from 2 to 4, from 3 to 4, from 0 to 3, from 1 to 3, from 2 to 3, from 0 to 2, from 1 to 2, from 0 to 1). In some embodiments, z can be 0, 5, 10, 15, 20, 25, 30, 35, 40, 45, 50, 55, 60, 65, 70, 75, 80, 85, 90, 95, or 100. In some embodiments, z can be 0.

In some embodiments, z is greater than 1. In some embodiments, z is greater than 5, greater than 10, greater than 15, greater than 20, greater than 25, greater than 30, greater than 35, greater than 40, greater than 45, greater than 50, greater than 55, greater than 60, greater than 65, greater than 70, greater than 75, greater than 80, greater than 85, greater than 90, or greater than 95. In some embodiments, when Q is hydrogen, z can be at least 6 (e.g., at least 8, at least 10, at least 15, at least 20, at least 30, at least 45, at least 50, at least 55, at least 60, at least 65, at least 70, at least 75, at least 80, at least 85, at least 90, or at least 95).

In some embodiments, R² can be —CH₂R³. In some embodiments, R² is absent. In some embodiments, R² is —((BO)ₓ—(PO)_{y}-(EO)_{z}-)ₘ-A. In some embodiments, R³ is —C(O)O-M⁺, or —C(O)OH. In some embodiments, R³ is C₁-C₁₀ alkoxy. In some embodiments, R³ is aryloxy. In some embodiments, p is 3. In some embodiments, p is 2. In some embodiments, m is 2.

In some embodiments, the compound of Formula IX can be selected from:

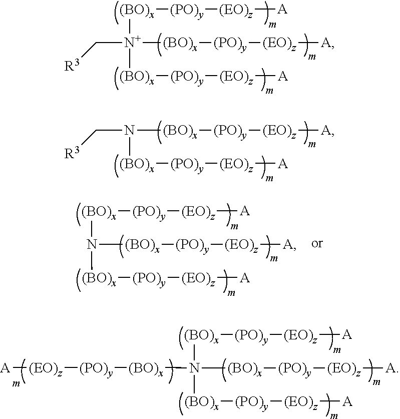

In some embodiments, the compound of Formula IX can be selected from:

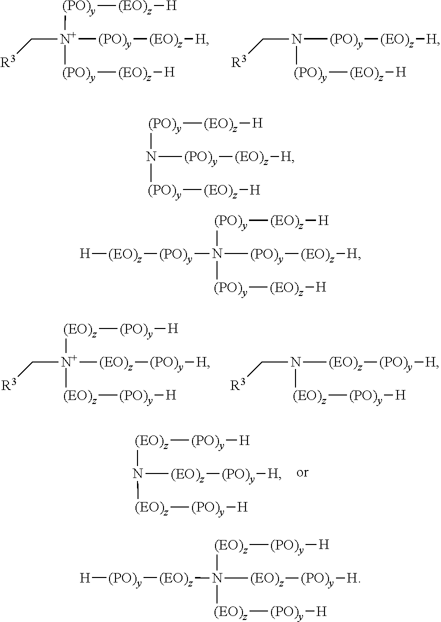

### Composition

As described above, the polyol alkoxylate surfactant described herein can be used in EOR formulations to provide aqueous stability and ultra-low interfacial tension region in heavy oils.

Accordingly, also provided are aqueous compositions for use in EOR that comprise the polyol alkoxylate surfactant described herein (e.g., a compound of Formula I). For example, provided herein are aqueous composition that comprise a polyol alkoxylate surfactant described herein (e.g., a compound of Formula I) and water. Additional components, including viscosity-enhancing water-soluble polymers, alkali agents, additional surfactants, co-solvents, and combinations thereof, can be present in the aqueous compositions.

In some embodiments, the aqueous composition can further comprise a surfactant. A surfactant, as used herein, is a compound within the aqueous composition that functions as a surface active agent when the aqueous composition is in contact with a crude oil (e.g., an unrefined petroleum). The surfactant can act to lower the interfacial tension and/or surface tension of the unrefined petroleum. In some embodiments, the surfactant and the compound of Formula I are present in synergistic surface active amounts. A “synergistic surface active amount,” as used herein, means that a compound of Formula I and the surfactant are present in amounts in which the oil surface activity (interfacial tension lowering effect and/or surface tension lowering effect on crude oil when the aqueous composition is added to the crude oil) of the compound and surfactant combined is greater than the additive oil surface activity of the surfactant individually and the compound individually. In some cases, the oil surface activity of the compound and surfactant combination is 10%, 20%, 30%, 40%, 50%, 60%, 70%, 80%, 90% or 100% more than the additive oil surface activity of the surfactant individually and the compound individually. In some embodiments, the oil surface activity of the compound and surfactant combination is 2, 3, 4, 5, 6, 7, 8, 9 or 10 times more than the additive oil surface activity of the surfactant individually and the compound individually.

In another embodiment, the compound and surfactant are present in a surfactant stabilizing amount. A “surfactant stabilizing amount” means that the compound and the surfactant are present in an amount in which the surfactant degrades at a slower rate in the presence of the compound than in the absence of the compound, and/or the compound degrades at a slower rate in the presence of the surfactant than in the absence of the surfactant. The rate of degradation may be 10%, 20%, 30%, 40%, 50%, 60%, 70%, 80%, 90% or 100% slower. In some embodiments, the rate of degradation is 2, 3, 4, 5, 6, 7, 8, 9 or 10 times slower.

In another embodiment, the compound and surfactant are present in a synergistic solubilizing amount. A “synergistic solubilizing amount” means that the compound and the surfactant are present in an amount in which the compound is more soluble in the presence of the surfactant than in the absence of the surfactant, and/or the surfactant is more soluble in the presence of the compound than in the absence of the compound. The solubilization may be 10%, 20%, 30%, 40%, 50%, 60%, 70%, 80%, 90% or 100% higher. In some embodiment, the solubilization is 2, 3, 4, 5, 6, 7, 8, 9 or 10 times higher. In some embodiments, the compound is present in an amount sufficient to increase the solubility of the surfactant in the aqueous composition relative to the absence of the compound. In other words, in the presence of a sufficient amount of the compound, the solubility of the surfactant in the aqueous composition is higher than in the absence of the compound. In other embodiments, the surfactant is present in an amount sufficient to increase the solubility of the compound in the aqueous composition relative to the absence of the surfactant. Thus, in the presence of a sufficient amount of the surfactant the solubility of the compound in the aqueous solution is higher than in the absence of the surfactant.

In some embodiments, a single type of surfactant is in the aqueous composition. In other embodiments, a surfactant can comprise a blend of surfactants (e.g., a combination of two or more surfactants). The surfactant blend can comprise a mixture of a plurality of surfactant types. For example, the surfactant blend can include at least two surfactant types, at least three surfactant types, at least four surfactant types, at least five surfactant types, at least six surfactant types, or more. In some embodiments, the surfactant blend can include from two to six surfactant types (e.g., from two to five surfactant types, from two to four surfactant types, from two to three surfactant types, from three to six surfactant types, or from three to five surfactant types). The surfactant types can be independently different (e.g., anionic or cationic surfactants; two anionic surfactants having a different hydrocarbon chain length but are otherwise the same; a sulfate and a sulfonate surfactant that that the same hydrocarbon chain length and are otherwise the same, etc.). Therefore, a person having ordinary skill in the art will immediately recognize that the terms “surfactant” and “surfactant type(s)” have the same meaning and can be used interchangeably.

In some embodiments, the surfactant can comprise an anionic surfactant, a non-ionic surfactant, a zwitterionic surfactant, a cationic surfactant, or a combination thereof. In some embodiments, the surfactant can comprise an anionic surfactant, a non-ionic surfactant, or a combination thereof. In some embodiments, the surfactant can comprise a plurality of anionic surfactants. In some embodiments, the surfactant can comprise a zwitterionic surfactant. “Zwitterionic” or “zwitterion” as used herein refers to a neutral molecule with a positive (or cationic) and a negative (or anionic) electrical charge at different locations within the same molecule. Examples of zwitterionic surfactants include without limitation betains and sultains.

The surfactant can be any appropriate surfactant useful in the field of enhanced oil recovery. For example, in some embodiments, the surfactant can comprise an internal olefin sulfonate (IOS), an alpha olefin sulfonate (AOS), an alkyl aryl sulfonate (ARS), an alkane sulfonate, a petroleum sulfonate, an alkyl diphenyl oxide (di)sulfonate, an alcohol sulfate, an alkoxy sulfate, an alkoxy sulfonate, an alcohol phosphate, an alkoxy phosphate, a sulfosuccinate ester, an alcohol ethoxylate, an alkyl phenol ethoxylate, a quaternary ammonium salt, a betaine or sultaine. The surfactant as provided herein, can also be a soap.

In embodiments, the surfactant can comprise an anionic surfactant. For example, the surfactant can comprise an anionic surfactant selected from the group consisting of alkoxy carboxylate surfactants, alkoxy sulfate surfactants, alkoxy sulfonate surfactants, alkyl sulfonate surfactants, aryl sulfonate surfactants, olefin sulfonate surfactants, and combinations thereof. In embodiments, the anionic surfactant can comprise an anionic surfactant blend. Where the anionic surfactant is an anionic surfactant blend, the aqueous composition includes a plurality (i.e., more than one) type of anionic surfactant.

Suitable surfactants are disclosed, for example, in U.S. Pat. Nos. 3,811,504, 3,811,505, 3,811,507, 3,890,239, 4,463,806, 6,022,843, 6,225,267, and 7,629,299; International Patent Application Publication Nos. WO/2008/079855, WO/2012/027757, WO/2016/145164, and WO/2011/094442; as well as U.S. Patent Application Publication Nos. 2005/0199395, 2006/0185845, 2006/018486, 2009/0270281, 2011/0046024, 2011/0100402, 2011/0190175, 2007/0191633, 2010/004843. 2011/0201531, 2011/0190174, 2011/0071057, 2011/0059873, 2011/0059872, 2011/0048721, 2010/0319920, 2010/0292110, and 2013/0281327, all of which are incorporated herein by reference in their entirety. Additional suitable surfactants are surfactants known to be used in enhanced oil recovery methods, including those discussed in D. B. Levitt, A. C. Jackson, L. Britton and G. A. Pope, “Identification and Evaluation of High-Performance EOR Surfactants,” SPE IX89, conference contribution for the SPE Symposium on Improved Oil Recovery Annual Meeting, Tulsa, Okla., Apr. 24-26, 2006.

A person having ordinary skill in the art will immediately recognize that many surfactants are commercially available as blends of related molecules (e.g., IOS and ABS surfactants). Thus, where a surfactant is present within a composition provided herein, a person of ordinary skill would understand that the surfactant might be a blend of a plurality of related surfactant molecules (as described herein and as generally known in the art).

In some embodiments, the total surfactant concentration (i.e., the compound of Formula I and one or more surfactants within the aqueous compositions provided herein) is from about 0.05% w/w to about 10% w/w. In other embodiments, the total surfactant concentration in the aqueous composition is from about 0.25% w/w to about 10% w/w. In other embodiments, the total surfactant concentration in the aqueous composition is about 0.5% w/w. In other embodiments, the total surfactant concentration in the aqueous composition is about 1.0% w/w. In other embodiments, the total surfactant concentration in the aqueous composition is about 1.25% w/w. In other embodiments, the total surfactant concentration in the aqueous composition is about 1.5% w/w. In other embodiments, the total surfactant concentration in the aqueous composition is about 1.75% w/w. In other embodiments, the total surfactant concentration in the aqueous composition is about 2.0% w/w. In other embodiments, the total surfactant concentration in the aqueous composition is about 2.5% w/w. In other embodiments, the total surfactant concentration in the aqueous composition is about 3.0% w/w. In other embodiments, the total surfactant concentration in the aqueous composition is about 3.5% w/w. In other embodiments, the total surfactant concentration in the aqueous composition is about 4.0% w/w. In other embodiments, the total surfactant concentration in the aqueous composition is about 4.5% w/w. In other embodiments, the total surfactant concentration in the aqueous composition is about 5.0% w/w. In other embodiments, the total surfactant concentration in the aqueous composition is about 5.5% w/w. In other embodiments, the total surfactant concentration in the aqueous composition is about 6.0% w/w. In other embodiments, the total surfactant concentration in the aqueous composition is about 6.5% w/w. In other embodiments, the total surfactant concentration in the aqueous composition is about 7.0% w/w. In other embodiments, the total surfactant concentration in the aqueous composition is about 7.5% w/w. In other embodiments, the total surfactant concentration in the aqueous composition is about 8.0% w/w. In other embodiments, the total surfactant concentration in the aqueous composition is about 9.0% w/w. In other embodiments, the total surfactant concentration in the aqueous composition is about 10% w/w.

In some embodiments, the concentration of the compound of Formula I is about 0.1%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.15%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.20%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.25%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.30%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.35%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.40%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.45%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.50%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.55%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.60%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.65%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.70%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.75%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.80%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.85%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.90%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 0.95%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 1.0%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 1.25%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 1.50%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 1.75%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 2%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 3%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 4%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the concentration of the compound of Formula I is about 5%. In some further embodiments, the concentration of the surfactant is about 0.05%. In some further embodiments, the concentration of the surfactant is about 0.10%. In some further embodiments, the concentration of the surfactant is about 0.15%. In some further embodiments, the concentration of the surfactant is about 0.20%. In some further embodiments, the concentration of the surfactant is about 0.25%. In some further embodiments, the concentration of the surfactant is about 0.30%. In some further embodiments, the concentration of the surfactant is about 0.35%. In some further embodiments, the concentration of the surfactant is about 0.40%. In some further embodiments, the concentration of the surfactant is about 0.45%. In some further embodiments, the concentration of the surfactant is about 0.50%. In some further embodiments, the concentration of the surfactant is about 0.55%. In some further embodiments, the concentration of the surfactant is about 0.60%. In some further embodiments, the concentration of the surfactant is about 0.65%. In some further embodiments, the concentration of the surfactant is about 0.70%. In some further embodiments, the concentration of the surfactant is about 0.75%. In some further embodiments, the concentration of the surfactant is about 0.80%. In some further embodiments, the concentration of the surfactant is about 0.85%. In some further embodiments, the concentration of the surfactant is about 0.90%. In some further embodiments, the concentration of the surfactant is about 0.95%. In some further embodiments, the concentration of the surfactant is about 1.0%. In some further embodiments, the concentration of the surfactant is about 1.25%. In some further embodiments, the concentration of the surfactant is about 1.5%. In some further embodiments, the concentration of the surfactant is about 1.75%. In some further embodiments, the concentration of the surfactant is about 2%. In some further embodiments, the concentration of the surfactant is about 3%. In some further embodiments, the concentration of the surfactant is about 4%. In some further embodiments, the concentration of the surfactant is about 5%.

In some embodiments, the aqueous compositions can further include a viscosity enhancing water-soluble polymer. In some embodiments, the water-soluble polymer may be a biopolymer such as xanthan gum or scleroglucan, a synthetic polymer such as polyacryamide, hydrolyzed polyarcrylamide or co-polymers of acrylamide and acrylic acid, 2-acrylamido 2-methyl propane sulfonate or N-vinyl pyrrolidone, a synthetic polymer such as polyethylene oxide, or any other high molecular weight polymer soluble in water or brine. In some embodiments, the polymer is polyacrylamide (PAM), partially hydrolyzed polyacrylamides (HPAM), and copolymers of 2-acrylamido-2-methylpropane sulfonic acid or sodium salt or mixtures thereof, and polyacrylamide (PAM) commonly referred to as AMPS copolymer and mixtures of the copolymers thereof. In one embodiment, the viscosity enhancing water-soluble polymer is polyacrylamide or a co-polymer of polyacrylamide. In one embodiment, the viscosity enhancing water-soluble polymer is a partially (e.g. 20%, 25%, 30%, 35%, 40%, 45%) hydrolyzed anionic polyacrylamide. In some further embodiment, the viscosity enhancing water-soluble polymer has a molecular weight of approximately about 8×10⁶ Daltons. In some other further embodiment, the viscosity enhancing water-soluble polymer has a molecular weight of approximately about 18×10⁶ Daltons. Non-limiting examples of commercially available polymers useful for the invention including embodiments provided herein are Florpaam 3330S and Florpaam 3360S. Molecular weights of the polymers may range from about 10,000 Daltons to about 20,000,000 Daltons. In some embodiments, the viscosity enhancing water-soluble polymer is used in the range of about 500 to about 5000 ppm concentration, such as from about 1000 to 2000 ppm (e.g., in order to match or exceed the reservoir oil viscosity under the reservoir conditions of temperature and pressure).

In some embodiments, the aqueous compositions can further include an alkali agent. An alkali agent as provided herein can be a basic, ionic salt of an alkali metal (e.g., lithium, sodium, potassium) or alkaline earth metal element (e.g., magnesium, calcium, barium, radium). Examples of suitable alkali agents include, for example, NaOH, KOH, LiOH, Na₂CO₃, NaHCO₃, Na-metaborate, Na silicate, Na orthosilicate, Na acetate or NH₄OH. The aqueous composition may include seawater, or fresh water from an aquifer, river or lake. In some embodiments, the aqueous composition includes hard brine water or soft brine water. In some further embodiments, the water is soft brine water. In some further embodiments, the water is hard brine water. Where the aqueous composition includes soft brine water, the aqueous composition can further include an alkaline agent. In soft brine water the alkaline agent can provide for enhanced soap generation from the active oils, lower surfactant adsorption to the solid material (e.g., rock) in the reservoir and increased solubility of viscosity enhancing water soluble polymers.

The alkali agent can be present in the aqueous composition at a concentration from about 0.1% w/w to about 10% w/w. The combined amount of alkali agent and compound provided herein (e.g., compound of Formula I) present in the aqueous composition provided herein can be approximately equal to or less than about 10% w/w. In some embodiments, the total concentration of alkali agent (i.e., the total amount of alkali agent within the aqueous compositions and emulsion compositions provided herein) in is from about 0.05% w/w to about 5% w/w. In other embodiments, the total alkali agent concentration in the aqueous composition is from about 0.25% w/w to about 5% w/w. In other embodiments, the total alkali agent concentration in the aqueous composition is about 0.5% w/w. In other embodiments, the total alkali agent concentration in the aqueous composition is about 0.75% w/w. In other embodiments, the total alkali agent concentration in the aqueous composition is about 1% w/w. In other embodiments, the total alkali agent concentration in the aqueous composition is about 1.25% w/w. In other embodiments, the total alkali agent concentration in the aqueous composition is about 1.50% w/w. In other embodiments, the total alkali agent concentration in the aqueous composition is about 1.75% w/w. In other embodiments, the total alkali agent concentration in the aqueous composition is about 2% w/w. In other embodiments, the total alkali agent concentration in the aqueous composition is about 2.25% w/w. In other embodiments, the total alkali agent concentration in the aqueous composition is about 2.5% w/w. In other embodiments, the total alkali agent concentration in the aqueous composition is about 2.75% w/w. In other embodiments, the total alkali agent concentration in the aqueous composition is about 3% w/w. In other embodiments, the total alkali agent concentration in the aqueous composition is about 3.25% w/w. In other embodiments, the total alkali agent concentration in the aqueous composition is about 3.5% w/w. In other embodiments, the total alkali agent concentration in the aqueous composition is about 3.75% w/w. In other embodiments, the total alkali agent concentration in the aqueous composition is about 4% w/w. In other embodiments, the total alkali agent concentration in the aqueous composition is about 4.25% w/w. In other embodiments, the total alkali agent concentration in the aqueous composition is about 4.5% w/w. In other embodiments, the total alkali agent concentration in the aqueous composition is about 4.75% w/w. In other embodiments, the total alkali agent concentration in the aqueous composition is about 5.0% w/w. In some embodiments, the alkali agent can be present in the aqueous compositions in an effective amount to afford an aqueous composition having a pH of from 10 to 12 (e.g., 10.5 to 11.5).

In some embodiments, the aqueous compositions can further include a co-solvent. In embodiments, the co-solvent is an alcohol, alcohol ethoxylate, glycol ether, glycols, or glycerol. The aqueous compositions provided herein may include more than one co-solvent. Thus, in embodiments, the aqueous composition includes a plurality of different co-solvents. Where the aqueous composition includes a plurality of different co-solvents, the different co-solvents can be distinguished by their chemical (structural) properties. For example, the aqueous composition may include a first co-solvent, a second co-solvent and a third co-solvent, wherein the first co-solvent is chemically different from the second and the third co-solvent, and the second co-solvent is chemically different from the third co-solvent. In embodiments, the plurality of different co-solvents includes at least two different alcohols (e.g., a C₁-C₆ alcohol and a C₁-C₄ alcohol). In embodiments, the aqueous composition includes a C₁-C₆ alcohol and a C₁-C₄ alcohol. In embodiments, the plurality of different co-solvents includes at least two different alkoxy alcohols (e.g., a C₁-C₆ alkoxy alcohol and a C₁-C₄ alkoxy alcohol). In embodiments, the aqueous composition includes a C₁-C₆ alkoxy alcohol and a C₁-C₄ alkoxy alcohol. In embodiments, the plurality of different co-solvents includes at least two co-solvents selected from the group consisting of alcohols, alkyl alkoxy alcohols and phenyl alkoxy alcohols. For example, the plurality of different co-solvents may include an alcohol and an alkyl alkoxy alcohol, an alcohol and a phenyl alkoxy alcohol, or an alcohol, an alkyl alkoxy alcohol and a phenyl alkoxy alcohol. The alkyl alkoxy alcohols or phenyl alkoxy alcohols provided herein have a hydrophobic portion (alkyl or aryl chain), a hydrophilic portion (e.g., an alcohol) and optionally an alkoxy (ethoxylate or propoxylate) portion. Thus, in embodiments, the co-solvent is an alcohol, alkoxy alcohol, glycol ether, glycol or glycerol. Suitable co-solvents are known in the art, and include, for example, co-solvents described in U.S. Patent Application Publication No. 2013/0281327 which is hereby incorporated herein in its entirety

In some embodiments, a co-solvent can be present in an amount sufficient to increase the solubility of the alkoxylated surfactant (when present) in the aqueous phase relative to the absence of the co-solvent. In other words, in the presence of a sufficient amount of the co-solvent, the solubility of the co-solvent in the aqueous phase is higher than in the absence of the co-solvent. In embodiments, the co-solvent can be present in an amount sufficient to increase the solubility of the surfactant in the aqueous phase relative to the absence of the co-solvent. Thus, in the presence of a sufficient amount of the co-solvent the solubility of the surfactant in the aqueous phase can be higher than in the absence of the co-solvent. In embodiments, the co-solvent can be present in an amount sufficient to decrease the viscosity of an emulsion formed from the composition relative to the absence of the co-solvent.

In some embodiments, the co-solvent can be a polyol alkoxylated co-solvent. The polyol alkoxylated co-solvent can be defined by Formula A below

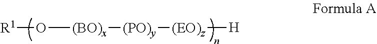

wherein

BO represents —CH₂—CH(ethyl)-O— or —CH₃CH(O—)CH₃;

PO represents —CH₂—CH(methyl)-O—;

EO represents —CH₂—CH₂—O—;

R¹ represents a C₁-C₈ alkyl group (e.g., a C₁-C₆ alkyl group) or a cyclic structure derived from alkoxylation of an alkyl monoglucoside, an alkyl polyglucoside, a monosaccharide, a disaccharide, or a polysaccharide; n is an integer from 2 to 6;

x is an integer from 0 to 5;

y is an integer from 1 to 10; and

z is an integer from 0 to 50.

In some embodiments, n can be an integer from 3 to 6, from 3 to 5, from 3 to 4, from 4 to 5, or from 4 to 6. In some embodiments, n can be an integer from 3 to 6. In some embodiments, n can be 3, 4, 5, or 6. In some embodiments, n is 3.

In some embodiments, R¹ can be a C₁-C₆ alkyl group, a C₁-C₅ alkyl, C₁-C₄ alkyl, C₁-C₃ alkyl, C₁-C₂ alkyl, C₂-C₆ alkyl, C₂-C₅ alkyl, C₂-C₄ alkyl, C₂-C₃ alkyl, C₃-C₆ alkyl, C₃-C₅ alkyl, C₃-C₄ alkyl, C₄-C₅ alkyl, C₄-C₆ alkyl, or C₅-C₆ alkyl group. In some embodiments, R¹ can be a C₃-C₆ alkyl group.

In some embodiments, the compound of Formula A can be defined by Formula B below

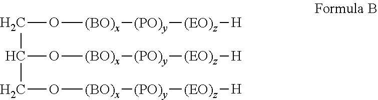

wherein

BO represents —CH₂—CH(ethyl)-O— or —CH₃CH(O—)CH₃;

PO represents —CH₂—CH(methyl)-O—;

EO represents —CH₂—CH₂—O—;

x is an integer from 0 to 5;

y is an integer from 1 to 10; and

z is an integer from 0 to 50.

In some embodiments, the compound of Formula A can be defined by Formula C below

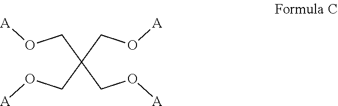

wherein

A represents —(BO)ₓ—(PO)_{y}-(EO)_{z}—H

BO represents —CH₂—CH(ethyl)-O— or —CH₃CH(O—)CH₃;

PO represents —CH₂—CH(methyl)-O—;

EO represents —CH₂—CH₂—O—;

x is an integer from 0 to 5;

y is an integer from 1 to 10; and

z is an integer from 0 to 50.

In some embodiments, the compound of Formula A can be defined by Formula C-a below

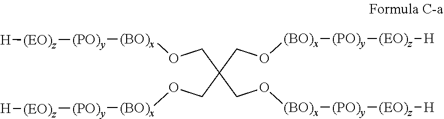

wherein

BO represents —CH₂—CH(ethyl)-O— or —CH₃CH(O—)CH₃;

PO represents —CH₂—CH(methyl)-O—;

EO represents —CH₂—CH₂—O—;

x is an integer from 0 to 5;

y is an integer from 1 to 10; and

z is an integer from 0 to 50.

In some embodiments, the compound of Formula A can be defined by Formula D below

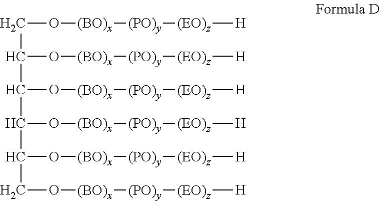

wherein

BO represents —CH₂—CH(ethyl)-O— or —CH₃CH(O—)CH₃;

PO represents —CH₂—CH(methyl)-O—;

EO represents —CH₂—CH₂—O—;

x is an integer from 0 to 5;

y is an integer from 1 to 10; and

z is an integer from 0 to 50.

In some embodiments, the compound of Formula A can be defined by Formula E below

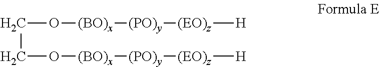

wherein

BO represents —CH₂—CH(ethyl)-O— or —CH₃CH(O—)CH₃;

PO represents —CH₂—CH(methyl)-O—;

EO represents —CH₂—CH₂—O—;

x is an integer from 0 to 5;

y is an integer from 1 to 10; and

z is an integer from 0 to 50.

In some embodiments, the compound of Formula A can be defined of Formula F below

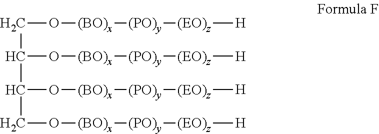

wherein

BO represents —CH₂—CH(ethyl)-O— or —CH₃CH(O—)CH₃;

PO represents —CH₂—CH(methyl)-O—;

EO represents —CH₂—CH₂—O—;

x is an integer from 0 to 5;

y is an integer from 1 to 10; and

z is an integer from 0 to 50.

In some embodiments, the compound of Formula A can be defined of Formula G below

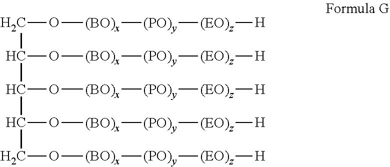

wherein

BO represents —CH₂—CH(ethyl)-O— or —CH₃CH(O—)CH₃;

PO represents —CH₂—CH(methyl)-O—;

EO represents —CH₂—CH₂—O—;

x is an integer from 0 to 5;

y is an integer from 1 to 10; and

z is an integer from 0 to 50.

In some embodiments, the compound of Formula A can be defined by Formula H below

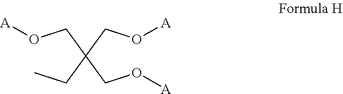

wherein

A represents —(BO)ₓ—(PO)_{y}-(EO)_{z}—H

BO represents —CH₂—CH(ethyl)-O— or —CH₃CH(O—)CH₃;

PO represents —CH₂—CH(methyl)-O—;

EO represents —CH₂—CH₂—O—;

x is an integer from 0 to 15;

y is an integer from 1 to 10; and

z is an integer from 0 to 50.

In some embodiments, the compound of Formula A can be defined by Formula D-a below

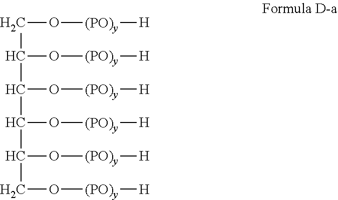

wherein

PO represents —CH₂—CH(methyl)-O—; and

y is an integer from 1 to 10.

In some embodiments, the compound of Formula A can be defined by Formula E-a below

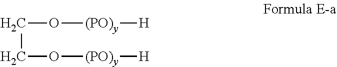

wherein

PO represents —CH₂—CH(methyl)-O—; and

y is an integer from 1 to 10.

In some embodiments, the compound of Formula A can be defined by Formula B-a below

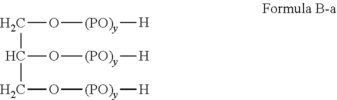

wherein

PO represents —CH₂—CH(methyl)-O—; and

y is an integer from 1 to 10.

In some embodiments, the compound of Formula A can be defined by Formula F-a below

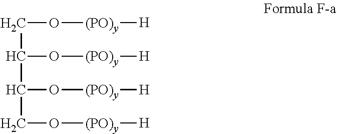

wherein

PO represents —CH₂—CH(methyl)-O—; and

y is an integer from 1 to 10.

In some embodiments, the compound of Formula A can be defined by Formula G-a below

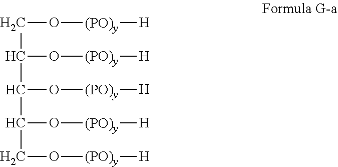

wherein

PO represents —CH₂—CH(methyl)-O—; and

y is an integer from 1 to 10.

In some embodiments, the compound of Formula I can be defined by Formula H-a below

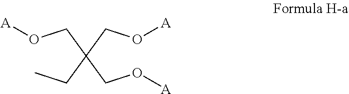

wherein

A represents —(PO)_{y}—H

PO represents —CH₂—CH(methyl)-O—; and

y is an integer from 1 to 10.

In some embodiments, the compound of Formula A can be defined by Formula B-b below

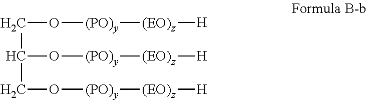

wherein

PO represents —CH₂—CH(methyl)-O—;

EO represents —CH₂—CH₂—O—;

y is an integer from 1 to 10; and

z is an integer from 0 to 50.

In some embodiments, the compound of Formula A can be defined by Formula C-b below

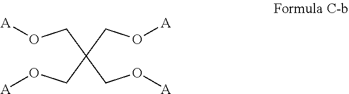

wherein

A represents —(PO)_{y}-(EO)_{z}—H

PO represents —CH₂—CH(methyl)-O—;

EO represents —CH₂—CH₂—O—;

y is an integer from 1 to 10; and

z is an integer from 0 to 50.

In some embodiments, the compound of Formula A can be defined by Formula C-c below

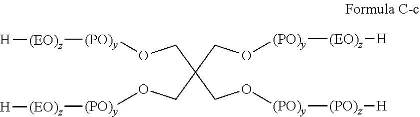

wherein

PO represents —CH₂—CH(methyl)-O—;

EO represents —CH₂—CH₂—O—;

y is an integer from 1 to 10; and

z is an integer from 0 to 50.

In some embodiments, the compound of Formula A can be defined by Formula D-b below

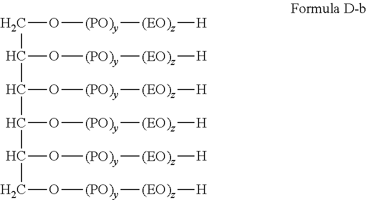

wherein

PO represents —CH₂—CH(methyl)-O—;

EO represents —CH₂—CH₂—O—;

y is an integer from 1 to 10; and

z is an integer from 0 to 50.

In some embodiments, the compound of Formula A can be defined by Formula E-b below

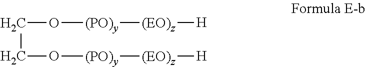

wherein

PO represents —CH₂—CH(methyl)-O—;

EO represents —CH₂—CH₂—O—;

y is an integer from 1 to 10; and

z is an integer from 0 to 50.

In some embodiments, the compound of Formula A can be defined of Formula F-b below

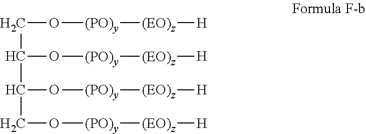

wherein

PO represents —CH₂—CH(methyl)-O—;

EO represents —CH₂—CH₂—O—;

y is an integer from 1 to 10; and

z is an integer from 0 to 50.

In some embodiments, the compound of Formula A can be defined of Formula G-b below

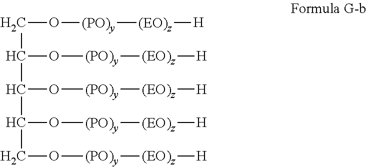

wherein

PO represents —CH₂—CH(methyl)-O—;

EO represents —CH₂—CH₂—O—;

y is an integer from 1 to 10; and

z is an integer from 0 to 50.

In some embodiments, the compound of Formula A can be defined by Formula H-b below

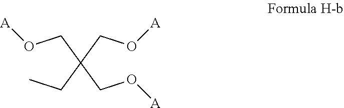

wherein

A represents —(PO)_{y}-(EO)_{z}—H

PO represents —CH₂—CH(methyl)-O—;

EO represents —CH₂—CH₂—O—;

y is an integer from 1 to 10; and

z is an integer from 0 to 50.

In some embodiments, the compound of Formula A can be defined by Formula B-c below

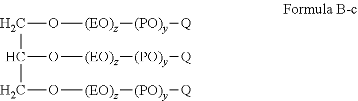

wherein

PO represents —CH₂—CH(methyl)-O—;

EO represents —CH₂—CH₂—O—;

Q is hydrogen;

y is an integer from 1 to 10; and

z is an integer from 0 to 50.

In some embodiments, the compound of Formula A can be defined by Formula C-e below

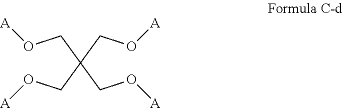

wherein

A represents -(EO)_{z}—(PO)_{y}-Q

PO represents —CH₂—CH(methyl)-O—;

EO represents —CH₂—CH₂—O—;

Q is hydrogen;

y is an integer from 1 to 10; and

z is an integer from 0 to 50.

In some embodiments, the compound of Formula A can be defined by Formula C-e below

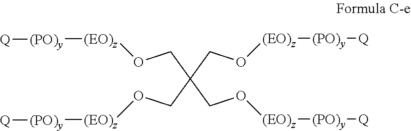

wherein

PO represents —CH₂—CH(methyl)-O—;

EO represents —CH₂—CH₂—O—;

Q is hydrogen;

y is an integer from 11 to 60; and

y is an integer from 1 to 10; and

z is an integer from 0 to 50.

In some embodiments, the compound of Formula A can be defined by Formula D-c below

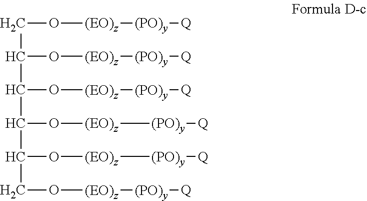

wherein

PO represents —CH₂—CH(methyl)-O—;

EO represents —CH₂—CH₂—O—;

Q is hydrogen;

y is an integer from 1 to 10; and

z is an integer from 0 to 50.

In some embodiments, the compound of Formula A can be defined by Formula E-c below

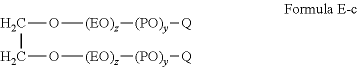

wherein

PO represents —CH₂—CH(methyl)-O—;

EO represents —CH₂—CH₂—O—;

Q is hydrogen;

y is an integer from 1 to 10; and

z is an integer from 0 to 50.

In some embodiments, the compound of Formula A can be defined of Formula F-c below

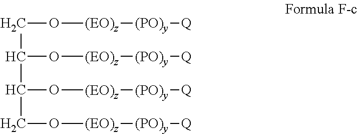

wherein

PO represents —CH₂—CH(methyl)-O—;

EO represents —CH₂—CH₂—O—;

Q is hydrogen;

y is an integer from 1 to 10; and

z is an integer from 0 to 50.

In some embodiments, the compound of Formula A can be defined of Formula G-c below

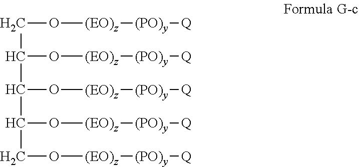

wherein

PO represents —CH₂—CH(methyl)-O—;

EO represents —CH₂—CH₂—O—;

Q is hydrogen;

y is an integer from 1 to 10; and

z is an integer from 0 to 50.

In some embodiments, the compound of Formula A can be defined by Formula H-c below

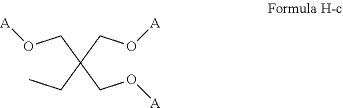

wherein

A represents -(EO)_{z}—(PO)_{y}-Q

PO represents —CH₂—CH(methyl)-O—;

EO represents —CH₂—CH₂—O—;

Q is hydrogen;

y is an integer from 1 to 10; and

z is an integer from 0 to 50.

In some embodiments, y can be an integer from 1 to 10, (e.g., from 1 to 2, from 1 to 3, from 1 to 4, from 1 to 5, from 1 to 6, from 1 to 7, from 1 to 8, from 1 to 9, from 2 to 3, from 2 to 4, from 2 to 5, from 2 to 6, from 2 to 7, from 2 to 8, from 2 to 9, from 2 to 10, from 3 to 4, from 3 to 5, from 3 to 6, from 3 to 7, from 3 to 8, from 3 to 9, from 3 to 10, from 4 to 5, from 4 to 6, from 4 to 7, from 4 to 8, from 4 to 9, from 4 to 10, from 5 to 6, from 5 to 7, from 5 to 8, from 5 to 9, from 5 to 10, from 6 to 7, from 6 to 8, from 6 to 9, from 6 to 10, from 7 to 8, from 7 to 9, from 7 to 10, from 8 to 9, from 8 to 10, or from 9 to 10). In some embodiments, y can be 1. In some embodiments, y can be 4. In some embodiments, y can be 6. In some embodiments, y can be 10.

In some embodiments, x can be an integer from 0 to 5 (e.g., from 0 to 5, from 1 to 5, from 2 to 5, from 3 to 5, from 4 to 5, from 0 to 4, from 1 to 4, from 2 to 4, from 3 to 4, from 0 to 3, from 1 to 3, from 2 to 3, from 0 to 2, from 1 to 2, or from 0 to 1). In some embodiments, x can be 0, 1, 2, 3, 4, or 5. In some embodiments, x can be 0.

In some embodiments, z can be an integer from 0 to 50 (e.g. from 0 to 50, from 1 to 50, from 5 to 50, from 10 to 50, from 15 to 50, from 20 to 50, from 25 to 50, from 30 to 50, from 40 to 50, from 0 to 40, from 1 to 40, from 5 to 40, from 10 to 40, from 15 to 40, from 20 to 40, from 25 to 40, from 30 to 40, from 0 to 35, from 1 to 35, from 5 to 35, from 10 to 35, from 15 to 35, from 20 to 35, from 25 to 35, from 0 to 30, from 1 to 30, from 5 to 30, from 10 to 30, from 15 to 30, from 20 to 30, from 25 to 30, from 0 to 25, from 1 to 25, from 5 to 25, from 10 to 25, from 15 to 25, from 20 to 25, from 0 to 20, from 1 to 20, from 5 to 20, from 10 to 20, from 15 to 20, from 0 to 15, from 1 to 15, from 5 to 15, from 10 to 15, from 0 to 10, from 1 to 10, from 5 to 10, from 0 to 5, from 1 to 5, from 2 to 5, from 3 to 5, from 4 to 5, from 0 to 4, from 1 to 4, from 2 to 4, from 3 to 4, from 0 to 3, from 1 to 3, from 2 to 3, from 0 to 2, from 1 to 2, from 0 to 1). In some embodiments, z can be 0, 5, 10, 15, 20, 25, 30, 35, 40, 45, or 50. In some embodiments, z can be 0.

In some embodiments, z is greater than 1. In some embodiments, z is greater than 5, greater than 10, greater than 15, greater than 20, greater than 25, greater than 30, greater than 35, greater than 40, or greater than 45.

In some embodiments, the co-solvent can be an amine based polyol alkoxylated co-solvent. The amine based polyol alkoxylated co-solvent can be defined by Formula J below

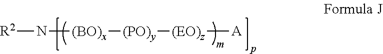

wherein

BO represents —CH₂—CH(ethyl)-O— or —CH₃CH(O—)CH₃;

PO represents —CH₂—CH(methyl)-O—;

EO represents —CH₂—CH₂—O—;

R² is absent, —((BO)ₓ—(PO)_{y}-(EO)_{z}-)ₘ-A, or —CH₂R³;

R³ is C₁-C₁₀ alkoxy, aryloxy, or —C(O)O⁻M⁺, or —C(O)OH;

M⁺, when present, is a cation;

m is an integer from 1 to 3;

p is an integer from 2 to 3;

A is H;

x is an integer from 0 to 5;

y is an integer from 1 to 10; and

z is an integer from 0 to 50,

wherein when R² is absent, p is 3.

In some embodiments, R² can be —CH₂R³. In some embodiments, R² is absent. In some embodiments, R² is —CH₂C(O)O⁻M⁺, or —CH₂C(O)OH.

In some embodiments, p is 3. In some embodiments, p is 2. In some embodiments, m is 2.

In some embodiments, the compound of Formula J can be selected from:

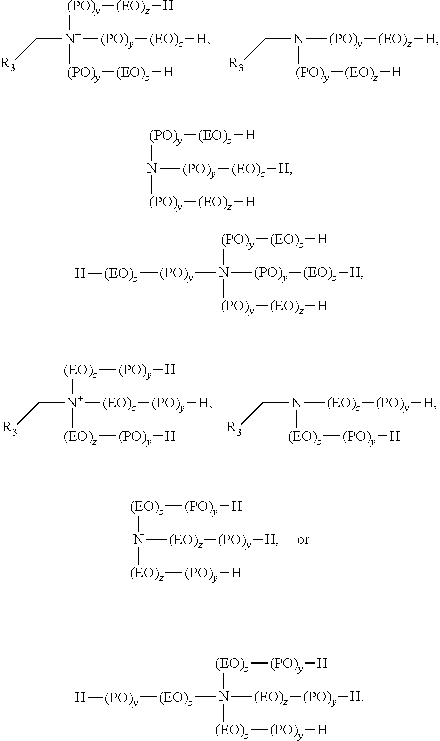

In some embodiments, y can be an integer from 1 to 10, (e.g., from 1 to 2, from 1 to 3, from 1 to 4, from 1 to 5, from 1 to 6, from 1 to 7, from 1 to 8, from 1 to 9, from 2 to 3, from 2 to 4, from 2 to 5, from 2 to 6, from 2 to 7, from 2 to 8, from 2 to 9, from 2 to 10, from 3 to 4, from 3 to 5, from 3 to 6, from 3 to 7, from 3 to 8, from 3 to 9, from 3 to 10, from 4 to 5, from 4 to 6, from 4 to 7, from 4 to 8, from 4 to 9, from 4 to 10, from 5 to 6, from 5 to 7, from 5 to 8, from 5 to 9, from 5 to 10, from 6 to 7, from 6 to 8, from 6 to 9, from 6 to 10, from 7 to 8, from 7 to 9, from 7 to 10, from 8 to 9, from 8 to 10, or from 9 to 10). In some embodiments, y can be 1. In some embodiments, y can be 4. In some embodiments, y can be 6. In some embodiments, y can be 10.

In some embodiments, x can be an integer from 0 to 5 (e.g., from 0 to 5, from 1 to 5, from 2 to 5, from 3 to 5, from 4 to 5, from 0 to 4, from 1 to 4, from 2 to 4, from 3 to 4, from 0 to 3, from 1 to 3, from 2 to 3, from 0 to 2, from 1 to 2, or from 0 to 1). In some embodiments, x can be 0, 1, 2, 3, 4, or 5. In some embodiments, x can be 0.

In some embodiments, z can be an integer from 0 to 50 (e.g. from 0 to 50, from 1 to 50, from 5 to 50, from 10 to 50, from 15 to 50, from 20 to 50, from 25 to 50, from 30 to 50, from 40 to 50, from 0 to 40, from 1 to 40, from 5 to 40, from 10 to 40, from 15 to 40, from 20 to 40, from 25 to 40, from 30 to 40, from 0 to 35, from 1 to 35, from 5 to 35, from 10 to 35, from 15 to 35, from 20 to 35, from 25 to 35, from 0 to 30, from 1 to 30, from 5 to 30, from 10 to 30, from 15 to 30, from 20 to 30, from 25 to 30, from 0 to 25, from 1 to 25, from 5 to 25, from 10 to 25, from 15 to 25, from 20 to 25, from 0 to 20, from 1 to 20, from 5 to 20, from 10 to 20, from 15 to 20, from 0 to 15, from 1 to 15, from 5 to 15, from 10 to 15, from 0 to 10, from 1 to 10, from 5 to 10, from 0 to 5, from 1 to 5, from 2 to 5, from 3 to 5, from 4 to 5, from 0 to 4, from 1 to 4, from 2 to 4, from 3 to 4, from 0 to 3, from 1 to 3, from 2 to 3, from 0 to 2, from 1 to 2, from 0 to 1). In some embodiments, z can be 0, 5, 10, 15, 20, 25, 30, 35, 40, 45, or 50. In some embodiments, z can be 0.

In some embodiments, z is greater than 1. In some embodiments, z is greater than 5, greater than 10, greater than 15, greater than 20, greater than 25, greater than 30, greater than 35, greater than 40, or greater than 45.

In other embodiments, the aqueous composition can be substantially free of co-solvents (e.g., the composition can include less than 0.05% by weight additional co-solvents, based on the total weight of the composition).

In some embodiments, the aqueous composition can further include a gas. For instance, the gas may be combined with the aqueous composition to reduce its mobility by decreasing the liquid flow in the pores of the solid material (e.g., rock). In some embodiments, the gas may be supercritical carbon dioxide, nitrogen, natural gas or mixtures of these and other gases.

In some embodiments, the aqueous composition can have a pH of at least 7 (e.g., a pH of at least 7.5, a pH of at least 8, a pH of at least 8.5, a pH of at least 9, a pH of at least 9.5, a pH of at least 10, a pH of at least 10.5, a pH of at least 11, a pH of at least 11.5, or a pH of at least 12.5). In some embodiments, the aqueous composition can have a pH of 13 or less (e.g., a pH of 12.5 or less, a pH of 12 or less, a pH of 11.5 or less, a pH of 11 or less, a pH of 10.5 or less, a pH of 10 or less, a pH of 9.5 or less, a pH of 9 or less, a pH of 8.5 or less, a pH of 8 or less, or a pH of 7.5 or less).

The aqueous composition can have a pH ranging from any of the minimum values described above to any of the maximum values described above. For example, the aqueous composition can have a pH of from 7 to 13 (e.g., from 10 to 12, or from 10.5 to 11.5).

In some embodiments, the aqueous composition can have a salinity of at least 5,000 ppm. In other embodiments, the aqueous composition has a salinity of at least 50,000 ppm. In other embodiments, the aqueous composition has a salinity of at least 100,000 ppm. In other embodiments, the aqueous composition has a salinity of at least 250,000 ppm. The total range of salinity (total dissolved solids in the brine) is 100 ppm to saturated brine (about 260,000 ppm). The aqueous composition may include seawater, brine or fresh water from an aquifer, river or lake. The aqueous combination may further include salt to increase the salinity. In some embodiments, the salt is NaCl, KCl, CaCl₂, MgCl₂, CaSO₄, Na acetate or Na₂CO₃.

In some embodiments, the aqueous composition can have a temperature of at least 20° C. (e.g., at least 30° C., at least 40° C., at least 50° C., at least 60° C., at least 70° C., at least 80° C., at least 90° C., at least 100° C., or at least 110° C.). The aqueous composition can have a temperature of 120° C. or less (e.g., 110° C. or less, 100° C. or less, 90° C. or less, 80° C. or less, 70° C. or less, 60° C. or less, 50° C. or less, 40° C. or less, or 30° C. or less).

The aqueous composition can have a temperature ranging from any of the minimum values described above to any of the maximum values described above. For example, the aqueous composition can have a temperature of from 20° C. to 120° C. (e.g., from 50° C. to 120° C., or from 80° C. to 120° C.).

In some embodiments, the aqueous composition can have a viscosity of between 20 mPas and 100 mPas at 20° C. The viscosity of the aqueous solution may be increased from 0.3 mPas to 1, 2, 10, 20, 100 or even 1000 mPas by including a water-soluble polymer. As mentioned above, the apparent viscosity of the aqueous composition may be increased with a gas (e.g., a foam forming gas) as an alternative to the water-soluble polymer.

Also provided are emulsions comprising a compound described herein or an aqueous composition described herein and unrefined petroleum. In some embodiments, the emulsion composition can be a microemulsion. A “microemulsion” as referred to herein is a thermodynamically stable mixture of oil, water and surfactants that may also include additional components such as co-solvents, electrolytes, alkali and polymers. In contrast, a “macroemulsion” as referred to herein is a thermodynamically unstable mixture of oil and water that may also include additional components. The emulsion composition provided herein may be an oil-in-water emulsion, wherein a surfactant forms aggregates (e.g., micelles) where the hydrophilic part of the surfactant molecule(s) contacts the aqueous phase of the emulsion and the lipophilic part contacts the oil phase of the emulsion. Thus, in some embodiments, the surfactant(s) form part of the aqueous part of the emulsion. And in other embodiments, the surfactant(s) form part of the oil phase of the emulsion. In yet another embodiment, the surfactant(s) form part of an interface between the aqueous phase and the oil phase of the emulsion.

In other embodiments, the oil and water solubilization ratios are insensitive to the combined concentration of divalent metal cations (e.g., Ca²⁺ and Mg²⁺) within the emulsion composition. In other embodiments, the oil and water solubilization ratios are insensitive to the salinity of the water or to all of the specific electrolytes contained in the water. The term “insensitive” used in the context of this paragraph means that the solubilization ratio tends not to change (e.g., tends to remain constant) as the concentration of divalent metal cations and/or salinity of water changes. In some embodiments, the change in the solubilization ratios are less than 5%, 10%, 20%, 30%, 40%, or 50% over a divalent metal cation concentration range of 10 ppm, 100 ppm, 1000 ppm or 10,000 ppm. In another embodiment, the change in the solubilization ratios are less than 5%, 10%, 20%, 30%, 40%, or 50% over a salinity concentration range of 10 ppm, 100 ppm, 1000 ppm or 10,000 ppm.

Also provided herein are inverted polymer solutions, as well as methods of preparing the inverted polymer solutions from polymer compositions. In some embodiments, the polymer composition can comprise a liquid polymer (LP) composition comprising: one or more synthetic (co)polymers, an inversion package comprising a compound described herein, optionally one or more hydrophobic liquids, and optionally one or more surfactants. In other embodiments, the polymer composition can comprise a powder polymer suspended in an inversion package comprising a compound described herein and optionally a water soluble solvent, wherein the powder polymer and the inversion package are present at a weight ratio of powder polymer to inversion package of from 20:80 to 80:20.

### Polymer Compositions

Provided herein are polymer compositions that include a compound described herein. In some embodiments, the polymer composition can comprise a liquid polymer (LP) composition comprising: one or more synthetic (co)polymers, an inversion package comprising a compound described herein, optionally one or more hydrophobic liquids, and optionally one or more surfactants. In other embodiments, the polymer composition can comprise a powder polymer suspended in an inversion package comprising a compound described herein and optionally a water soluble solvent, wherein the powder polymer and the inversion package are present at a weight ratio of powder polymer to inversion package of from 20:80 to 80:20.

In some embodiments, LP compositions can comprise one or more synthetic (co)polymers (e.g., one or more acrylamide (co)polymers) and an inversion package dispersed or emulsified in one or more hydrophobic liquids. In some embodiments, the LP compositions can further comprise one or more surfactants (e.g., one or more emulsifying surfactants and one or more inverting surfactants). In some embodiments, the LP compositions can further comprise a small amount of water. For example, the LP compositions can further comprise less than 10% by weight (e.g., less than 5% by weight, less than 4% by weight, less than 3% by weight, less than 2.5% by weight, less than 2% by weight, or less than 1% by weight) water, based on the total weight of all the components of the LP composition. In certain embodiments, the LP compositions can be water-free or substantially water-free (i.e., the composition can include less than 0.5% by weight water, based on the total weight of the composition). The LP compositions can optionally include one or more additional components which do not substantially diminish the desired performance or activity of the composition. It will be understood by a person having ordinary skill in the art how to appropriately formulate the LP composition to provide necessary or desired features or properties.

In some embodiments, the LP composition can comprise one or more hydrophobic liquids; one or more synthetic co-polymers (e.g., acrylamide-(co)polymers); and an inversion package.

In some embodiments, the LP composition can comprise one or more hydrophobic liquids; one or more synthetic co-polymers (e.g., acrylamide-(co)polymers); an inversion package, and one or more surfactants (e.g., one or more emulsifier surfactants, and/or one or more inverting surfactants).

In some embodiments, the LP composition can comprise one or more hydrophobic liquids; one or more acrylamide-(co)polymers; an inversion package; one or more emulsifier surfactants; and one or more inverting surfactants.

In some embodiments, the LP compositions can comprise less than 10% by weight (e.g., less than 7% by weight, less than 5% by weight, less than 4% by weight, less than 3% by weight, less than 2.5% by weight, less than 2% by weight, or less than 1% by weight) water prior to inversion, based on the total weight of all the components of the LP composition. In certain embodiments, the LP composition, prior to inversion, comprises from 1% to 10% water by weight, or from 1% to 5% water by weight, based on the total amount of all components of the composition.

In some embodiments, the solution viscosity (SV) of a 0.1% solution of the LP composition can be greater than 3.0 cP, or greater than 5 cP, or greater than 7 cP. The SV of the LP composition can be selected based, at least in part, on the intended actives concentration of the inverted polymer solution, to provide desired performance characteristics in the inverted polymer solution. A liquid polymer composition with a lower or higher SV range may still provide desirable results, but may require changing the actives concentration of the inverted composition to achieve desired filter ratio and viscosity properties. For example, if the liquid polymer composition has a lower SV range, it may be desirable to increase the actives concentration of the inverted composition.

In some embodiments, the LP composition can comprise one or more synthetic (co)polymers (e.g., one or more acrylamide (co)polymers) and an inversion package comprising a compound described herein dispersed in one or more hydrophobic liquids. In these embodiments, the LP composition can comprise at least 39% inversion package by weight (e.g., at least 40% by weight, at least 45% by weight, at least 50% by weight, at least 55% by weight, at least 60% by weight, at least 65% by weight, at least 70% by weight, or at least 75% by weight), based on the total amount of all components of the composition. In some embodiments, the LP composition can comprise 80% by weight or less inversion package (e.g., 75% by weight or less, 70% by weight or less, 65% by weight or less, 60% by weight or less, 55% by weight or less, 50% by weight or less, 45% by weight or less, or 40% by weight or less), based on the total amount of all components of the composition.

The LP composition can comprise an amount of inversion package ranging from any of the minimum values described above to any of the maximum values described above. For example, in some embodiments, the LP composition can comprise from 39% to 80% by weight inversion package (e.g., from 39% to 60% by weight, or from 39% to 50% by weight), based on the total weight of the composition.

In some embodiments, the LP composition can comprise one or more synthetic (co)polymers (e.g., one or more acrylamide (co)polymers) and an inversion package comprising a compound described herein emulsified in one or more hydrophobic liquids. In these embodiments, the LP composition can comprise at least 10% inversion package by weight (e.g., at least 15% by weight, at least 20% by weight, at least 25% by weight, or at least 30% by weight), based on the total amount of all components of the composition. In some embodiments, the LP composition can comprise less than 38% by weight inversion package (e.g., less than 35% by weight, less than 30% by weight, less than 25% by weight, less than 20% by weight, or less than 15% by weight), based on the total amount of all components of the composition.

The LP composition can comprise an amount of inversion package ranging from any of the minimum values described above to any of the maximum values described above. For example, in some embodiments, the LP composition can comprise from 10% to 38% by weight inversion package (e.g., from 10% to 35% by weight, from 15% to 30% by weight, from 15% to 35% by weight, from 15% to 38% by weight, from 20% to 30% by weight, from 20% to 35% by weight, or from 20% to 38% by weight), based on the total weight of the composition.

In other embodiments, the polymer composition can comprise a powder polymer suspended in an inversion package comprising a compound described herein and optionally a water soluble solvent, wherein the powder polymer and the inversion package are present at a weight ratio of powder polymer to inversion package of from 20:80 to 80:20. Such polymer compositions are described, for example, in U.S. Pat. No. 9,909,053, which is hereby incorporated by reference in its entirety. In some embodiments the water soluble solvent has an HLB value of greater than or equal to 8. The water soluble solvent comprises a non-ionic surfactant, an anionic surfactant, or a combination thereof.

Further aspects of these polymer compositions are described below.

### Synthetic (Co)Polymers

In some embodiments, the polymer composition includes one or more synthetic (co)polymers, such as one or more acrylamide containing (co)polymers. As used herein, the terms “polymer,” “polymers,” “polymeric,” and similar terms are used in their ordinary sense as understood by one skilled in the art, and thus may be used herein to refer to or describe a large molecule (or group of such molecules) that contains recurring units. Polymers may be formed in various ways, including by polymerizing monomers and/or by chemically modifying one or more recurring units of a precursor polymer. A polymer may be a “homopolymer” comprising substantially identical recurring units formed by, e.g., polymerizing a particular monomer. A polymer may also be a “copolymer” comprising two or more different recurring units formed by, e.g., copolymerizing two or more different monomers, and/or by chemically modifying one or more recurring units of a precursor polymer. The term “terpolymer” may be used herein to refer to polymers containing three or more different recurring units. The term “polymer” as used herein is intended to include both the acid form of the polymer as well as its various salts.

In some embodiments, the one or more synthetic (co)polymers can be a polymer useful for enhanced oil recovery applications. The term “enhanced oil recovery” or “EOR” (also known as tertiary oil recovery), refers to a process for hydrocarbon production in which an aqueous injection fluid comprising at least a water soluble polymer is injected into a hydrocarbon bearing formation.

In some embodiments, the one or more synthetic (co)polymers comprise water-soluble synthetic (co)polymers. Examples of suitable synthetic (co)polymers include acrylic polymers, such as polyacrylic acids, polyacrylic acid esters, partly hydrolyzed acrylic esters, substituted polyacrylic acids such as polymethacrylic acid and polymethacrylic acid esters, polyacrylamides, partly hydrolyzed polyacrylamides, and polyacrylamide derivatives such as acrylamide tertiary butyl sulfonic acid (ATBS); copolymers of unsaturated carboxylic acids, such as acrylic acid or methacrylic acid, with olefins such as ethylene, propylene and butylene and their oxides; polymers of unsaturated dibasic acids and anhydrides such as maleic anhydride; vinyl polymers, such as polyvinyl alcohol (PVA), N-vinylpyrrolidone, and polystyrene sulfonate; and copolymers thereof, such as copolymers of these polymers with monomers such as ethylene, propylene, styrene, methylstyrene, and alkylene oxides. In some embodiments, the one or more synthetic (co)polymer can comprise polyacrylic acid (PAA), polyacrylamide (PAM), acrylamide tertiary butyl sulfonic acid (ATBS) (or AMPS, 2-acrylamido-2-methylpropane sulfonic acid), N-vinylpyrrolidone (NVP), polyvinyl alcohol (PVA), or a blend or copolymer of any of these polymers. Copolymers may be made of any combination above, for example, a combination of NVP and ATBS. In certain examples, the one or more synthetic (co)polymers can comprise acrylamide tertiary butyl sulfonic acid (ATBS) (or AMPS, 2-acrylamido-2-methylpropane sulfonic acid) or a copolymer thereof.

In some embodiments, the one or more synthetic (co)polymers can comprise acrylamide (co)polymers. In some embodiments, the one or more acrylamide (co)polymers comprise water-soluble acrylamide (co)polymers. In various embodiments, the acrylamide (co)polymers comprise at least 30% by weight, or at least 50% by weight acrylamide units with respect to the total amount of all monomeric units in the (co)polymer.

Optionally, the acrylamide-(co)polymers can comprise, besides acrylamide, at least one additional co-monomer. In example embodiments, the acrylamide-(co)polymer may comprise less than about 50%, or less than about 40%, or less than about 30%, or less than about 20% by weight of the at least one additional co-monomer. In some embodiments, the additional comonomer can be a water-soluble, ethylenically unsaturated, in particular monoethylenically unsaturated, comonomer. Suitable additional water-soluble comonomers include comonomers that are miscible with water in any ratio, but it is sufficient that the monomers dissolve sufficiently in an aqueous phase to copolymerize with acrylamide. In some cases, the solubility of such additional monomers in water at room temperature can be at least 50 g/L (e.g., at least 150 g/L, or at least 250 g/L).

Other suitable water-soluble comonomers can comprise one or more hydrophilic groups. The hydrophilic groups can be, for example, functional groups that comprise one or more atoms selected from the group of O—, N—, S—, and P-atoms. Examples of such functional groups include carbonyl groups >C—O, ether groups —O—, in particular polyethylene oxide groups —(CH₂—CH₂—O—)ₙ—, where n is preferably a number from 1 to 200, hydroxy groups —OH, ester groups —C(O)O—, primary, secondary or tertiary amino groups, ammonium groups, amide groups —C(O)—NH— or acid groups such as carboxyl groups —COOH, sulfonic acid groups —SO₃H, phosphonic acid groups —PO₃H₂ or phosphoric acid groups —OP(OH)₃.

Examples of monoethylenically unsaturated comonomers comprising acid groups include monomers comprising —COOH groups, such as acrylic acid or methacrylic acid, crotonic acid, itaconic acid, maleic acid or fumaric acid, monomers comprising sulfonic acid groups, such as vinylsulfonic acid, allylsulfonic acid, 2-acrylamido-2-methylpropanesulfonic acid, 2-methacrylamido-2-methylpropanesulfonic acid, 2-acrylamidobutanesulfonic acid, 3-acrylamido-3-methylbutanesulfonic acid or 2-acrylamido-2,4,4-trimethylpentanesulfonic acid, or monomers comprising phosphonic acid groups, such as vinylphosphonic acid, allylphosphonic acid, N-(meth)acrylamidoalkylphosphonic acids or (meth)acryloyloxyalkyl-phosphonic acids. Of course the monomers may be used as salts.

The —COOH groups in polyacrylamide-copolymers may not only be obtained by copolymerizing acrylic amide and monomers comprising —COOH groups but also by hydrolyzing derivatives of —COOH groups after polymerization. For example, the amide groups —CO—NH₂ of acrylamide may hydrolyze thus yielding —COOH groups.

Also to be mentioned are derivatives of acrylamide thereof, such as, for example, N-methyl(meth)acrylamide, N,N′-dimethyl(meth)acrylamide, and N-methylolacrylamide, N-vinyl derivatives such as N-vinylformamide, N-vinylacetamide, N-vinylpyrrolidone or N-vinylcaprolactam, and vinyl esters, such as vinyl formate or vinyl acetate. N-vinyl derivatives can be hydrolyzed after polymerization to vinylamine units, vinyl esters to vinyl alcohol units.

Other example comonomers include monomers comprising hydroxy and/or ether groups, such as, for example, hydroxyethyl(meth)acrylate, hydroxypropyl(meth)acrylate, allyl alcohol, hydroxyvinyl ethyl ether, hydroxyl vinyl propyl ether, hydroxyvinyl butyl ether or polyethyleneoxide(meth)acrylates.

Other example comonomers are monomers having ammonium groups, i.e monomers having cationic groups. Examples comprise salts of 3-trimethylammonium propylacrylamides or 2-trimethylammonium ethyl(meth)acrylates, for example the corresponding chlorides, such as 3-trimethylammonium propylacrylamide chloride (DIMAPAQUAT) and 2-trimethylammonium ethyl methacrylate chloride (MADAME-QUAT).

Other example monoethylenically unsaturated monomers include monomers which may cause hydrophobic association of the (co)polymers. Such monomers comprise besides the ethylenic group and a hydrophilic part also a hydrophobic part. Such monomers are disclosed for instance in WO 2012/069477, which is incorporated herein by reference in its entirety.

Other example comonomers include N-alkyl acrylamides and N-alkyl quaternary acrylamides, where the alkyl group comprises, for example, a C₂-C₂₈ alkyl group.

In certain embodiments, each of the one or more acrylamide-(co)polymers can optionally comprise crosslinking monomers, i.e. monomers comprising more than one polymerizable group. In certain embodiments, the one or more acrylamide-(co)polymers may optionally comprise crosslinking monomers in an amount of less than 0.5%, or 0.1%, by weight, based on the amount of all monomers.

In an embodiment, each of the one or more acrylamide-(co)polymers comprises at least one monoethylenically unsaturated comonomer comprising acid groups, for example monomers which comprise at least one group selected from —COOH, —SO₃H or —PO₃H₂. Examples of such monomers include but are not limited to acrylic acid, methacrylic acid, vinylsulfonic acid, allylsulfonic acid or 2-acrylamido-2-methylpropanesulfonic acid, particularly preferably acrylic acid and/or 2-acrylamido-2-methylpropanesulfonic acid and most preferred acrylic acid or the salts thereof. The amount of such comonomers comprising acid groups can be from 0.1% to 70%, from 1% to 50%, or from 10% to 50% by weight based on the amount of all monomers.

In an embodiment, each of the one or more acrylamide-(co)polymers comprise from 50% to 90% by weight of acrylamide units and from 10% to 50% by weight of acrylic acid units and/or their respective salts, based on the total weight of all the monomers making up the copolymer. In an embodiment, each of the one or more acrylamide-(co)polymers comprise from 60% to 80% by weight of acrylamide units and from 20% to 40% by weight of acrylic acid units, based on the total weight of all the monomers making up the copolymer.

In some embodiments, the one or more synthetic (co)polymers (e.g., the one or more acrylamide (co)polymers) are in the form of particles, which are dispersed in the emulsion or LP. In some embodiments, the particles of the one or more synthetic (co)polymers can have an average particle size of from 0.4 μm to 5 μm, or from 0.5 μm to 2 μm. Average particle size refers to the d₅₀ value of the particle size distribution (number average) as measured by laser diffraction analysis.

In some embodiments, the one or more synthetic (co)polymers (e.g., the one or more acrylamide (co)polymers) can have a weight average molecular weight (M_{w}) of from 5,000,000 g/mol to 30,000,000 g/mol; from 10,000,000 g/mol to 25,000,000 g/mol; or from 15,000,000 g/mol to 25,000,000 g/mol.

In some embodiments, the LP composition can comprise one or more synthetic (co)polymers (e.g., one or more acrylamide (co)polymers) and an inversion package comprising a compound described herein dispersed in one or more hydrophobic liquids. In these embodiments, the LP composition can comprise at least 39% polymer by weight (e.g., at least 40% by weight, at least 45% by weight, at least 50% by weight, at least 55% by weight, at least 60% by weight, at least 65% by weight, at least 70% by weight, or at least 75% by weight), based on the total amount of all components of the composition. In some embodiments, the LP composition can comprise 80% by weight or less polymer (e.g., 75% by weight or less, 70% by weight or less, 65% by weight or less, 60% by weight or less, 55% by weight or less, 50% by weight or less, 45% by weight or less, or 40% by weight or less), based on the total amount of all components of the composition.

The LP composition can comprise an amount of polymer ranging from any of the minimum values described above to any of the maximum values described above. For example, in some embodiments, the LP composition can comprise from 39% to 80% by weight polymer (e.g., from 39% to 60% by weight, from 39% to 50% by weight, from 40% to 60% by weight, or from 45% to 55% by weight), based on the total weight of the composition (before dilution).

In some embodiments, the LP composition can comprise one or more synthetic (co)polymers (e.g., one or more acrylamide (co)polymers) and an inversion package comprising a compound described herein emulsified in one or more hydrophobic liquids. In these embodiments, the LP composition can comprise at least 10% polymer by weight (e.g., at least 15% by weight, at least 20% by weight, at least 25% by weight, or at least 30% by weight), based on the total amount of all components of the composition. In some embodiments, the LP composition can comprise less than 38% by weight polymer (e.g., less than 35% by weight, less than 30% by weight, less than 25% by weight, less than 20% by weight, or less than 15% by weight), based on the total amount of all components of the composition.

The LP composition can comprise an amount of polymer ranging from any of the minimum values described above to any of the maximum values described above. For example, in some embodiments, the LP composition can comprise from 10% to 38% by weight polymer (e.g., from 10% to 35% by weight polymer, from 15% to 30% by weight polymer, from 15% to 35% by weight polymer, from 15% to 38% by weight polymer, from 20% to 30% by weight polymer, from 20% to 35% by weight polymer, or from 20% to 38% by weight polymer), based on the total weight of the composition (before dilution).

### Additional Surfactants

### Emulsifying Surfactants

In some embodiments, the LP composition can include one or more emulsifying surfactants. In some embodiments, the one or more emulsifying surfactants are surfactants capable of stabilizing water-in-oil-emulsions. Emulsifying surfactants, among other things, in the emulsion, lower the interfacial tension between the water and the water-immiscible liquid so as to facilitate the formation of a water-in-oil polymer emulsion. It is known in the art to describe the capability of surfactants to stabilize water-in-oil-emulsions or oil-in-water emulsions by using the so called “HLB-value” (hydrophilic-lipophilic balance). The HLB-value usually is a number from 0 to 20. In surfactants having a low HLB-value the lipophilic parts of the molecule predominate and consequently they are usually good water-in-oil emulsifiers. In surfactants having a high HLB-value the hydrophilic parts of the molecule predominate and consequently they are usually good oil-in-water emulsifiers. In some embodiments, the one or more emulsifying surfactants are surfactants having an HLB-value of from 2 to 10, or a mixture of surfactant having an HLB-value of from 2 to 10.

Examples of suitable emulsifying surfactants include, but are not limited to, sorbitan esters, in particular sorbitan monoesters with C₁₂-C₁₈-groups such as sorbitan monolaurate (HLB approx. 8.5), sorbitan monopalmitate (HLB approx. 7.5), sorbitan monostearate (HLB approx. 4.5), sorbitan monooleate (HLB approx. 4); sorbitan esters with more than one ester group such as sorbitan tristearate (HLB approx. 2), sorbitan trioleate (HLB approx. 2); ethoxylated fatty alcohols with 1 to 4 ethyleneoxy groups, e.g. polyoxyethylene (4) dodecylether ether (HLB value approx. 9), polyoxyethylene (2) hexadecyl ether (HLB value approx. 5), and polyoxyethylene (2) oleyl ether (HLB value approx. 4).

Exemplary emulsifying surfactants include, but are not limited to, emulsifiers having HLB values of from 2 to 10 (e.g., less than 7). Suitable such emulsifiers include the sorbitan esters, phthalic esters, fatty acid glycerides, glycerine esters, as well as the ethoxylated versions of the above and any other well known relatively low HLB emulsifier. Examples of such compounds include sorbitan monooleate, the reaction product of oleic acid with isopropanolamide, hexadecyl sodium phthalate, decyl sodium phthalate, sorbitan stearate, ricinoleic acid, hydrogenated ricinoleic acid, glyceride monoester of lauric acid, glyceride monoester of stearic acid, glycerol diester of oleic acid, glycerol triester of 12-hydroxystearic acid, glycerol triester of ricinoleic acid, and the ethoxylated versions thereof containing 1 to 10 moles of ethylene oxide per mole of the basic emulsifier. Thus, any emulsifier can be utilized which will permit the formation of the initial emulsion and stabilize the emulsion during the polymerization reaction. Examples of emulsifying surfactants also include modified polyester surfactants, anhydride substituted ethylene copolymers, N,N-dialkanol substituted fatty amides, and tallow amine ethoxylates.

In an embodiment, the inverse emulsion or LP composition comprises from 0% to 5% by weight (e.g., from 0.05% to 5%, from 0.1% to 5%, or from 0.5% to 3% by weight) of the one or more emulsifying surfactants, based on the total weight of the composition. These emulsifying surfactants can be used alone or in mixtures. In some embodiments, the inverse emulsion or LP composition can comprise less than 5% by weight (e.g., less than 4% by weight, or less than 3% by weight) of the one or more emulsifying surfactants, based on the total weight of the composition.

### Inverting Surfactants

In some embodiments, the LP composition optionally can include one or more inverting surfactants. In some embodiments, the one or more emulsifying surfactants are surfactants which can be used to accelerate the formation of an inverted composition (e.g., an inverted (co)polymer solution) after mixing the inverse emulsion or LP composition with an aqueous fluid.

Suitable inverting surfactants are known in the art, and include, for example, nonionic surfactants comprising a hydrocarbon group and a polyalkylenoxy group of sufficient hydrophilic nature. In some cases, nonionic surfactants defined by the general formula R¹—O—(CH(R²)—CH₂—O)ₙH (I) can be used, wherein R¹ is a C₈-C₂₂-hydrocarbon group, such as an aliphatic C₁₀-C₁₈-hydrocarbon group, n is a number of ≥4, preferably ≥6, and R² is H, methyl or ethyl, with the proviso that at least 50% of the groups R² are H. Examples of such surfactants include polyethoxylates based on C₁₀-C₁₈-alcohols such as C_{12/14}-, C_{14/18}- or C_{16/18}-fatty alcohols, C₁₃- or C_{13/15}-oxoalcohols. The HLB-value can be adjusted by selecting the number of ethoxy groups. Specific examples include tridecylalcohol ethoxylates comprising from 4 to 14 ethylenoxy groups (e.g., tridecyalcohol-8 EO (HLB-value approx. 13-14)) or C_{12/14} fatty alcohol ethoxylates (e.g., C_{12/14}-8 EO (HLB-value approx. 13)). Examples of emulsifying surfactants also include modified polyester surfactants, anhydride substituted ethylene copolymers, N,N-dialkanol substituted fatty amides, and tallow amine ethoxylates.

Other suitable inverting surfactants include anionic surfactants, such as, for example, surfactants comprising phosphate or phosphonic acid groups.

In some embodiments, the one or more inverting surfactants can comprise polyoxyethylene sorbitol tetraoleate, C₁₂₋₁₄ branched ethoxylated alcohol, polyethylene glycol monoleate. In certain embodiments, the one or more inverting surfactants can comprise from 1 to 20 mole % polyoxyethylene sorbitol tetraoleate, from 60 to 80 mole % C₁₂₋₁₄ branched ethoxylated alcohol and about 15 to about 25 mole % polyethylene glycol monoleate.

In some embodiments, the amount of the one or more inverting surfactants in the inverse emulsion or LP composition is from 1% to 10% (e.g., from 1% to 5%) by weight. based on the total amount of all components of the inverse emulsion or LP composition.

In certain embodiments, the one or more inverting surfactants can be added to the inverse emulsion or LP composition directly after preparation of the composition comprising the one or more acrylamide (co)polymers dispersed in one or more hydrophobic liquids, and optionally the one or more emulsifying surfactants (i.e., the inverse emulsion or liquid dispersion polymer composition which is transported from the location of manufacture to the location of use already comprises the one or more inverting surfactants).

In another embodiment the one or more inverting surfactants may be added to the inverse emulsion or LP composition at the location of use (e.g., at an off-shore production site).

### Hydrophobic Liquid

In some embodiments, the LP composition can include one or more hydrophobic liquids. In some cases, the one or more hydrophobic liquids can be organic hydrophobic liquids. In some embodiments, the one or more hydrophobic liquids each have a boiling point at least 100° C. (e.g., at least 135° C., or at least 180° C.). If the organic liquid has a boiling range, the term “boiling point” refers to the lower limit of the boiling range.

In some embodiments, the one or more hydrophobic liquids can be aliphatic hydrocarbons, aromatic hydrocarbons, or mixtures thereof. Examples of hydrophobic liquids include but are not limited to water-immiscible solvents, such as paraffin hydrocarbons, naphthene hydrocarbons, aromatic hydrocarbons, olefins, oils, stabilizing surfactants, and mixtures thereof. The paraffin hydrocarbons can be saturated, linear, or branched paraffin hydrocarbons. Examples of suitable aromatic hydrocarbons include, but are not limited to, toluene and xylene. In certain embodiments, the hydrophobic liquid can comprise an oil, for example, a vegetable oil, such as soybean oil, rapeseed oil, canola oil, or a combination thereof, and any other oil produced from the seed of any of several varieties of the rape plant.

In some embodiments, the amount of the one or more hydrophobic liquids in the inverse emulsion or LP composition is from 20% to 60%, from 25% to 54%, or from 35% to 54% by weight, based on the total amount of all components of the LP composition.

### Other Components

Optional further components can be added to the polymer compositions described herein. Examples of such components comprise radical scavengers, oxygen scavengers, chelating agents, biocides, stabilizers, or sacrificial agents.

### Process Stabilizing Agents

In some embodiments, the polymer composition can optionally include one or more process stabilizing agents. The process stabilizing agents aim at stabilizing the dispersion of the particles of polyacrylamide-(co)polymers in the organic, hydrophobic phase and optionally also at stabilizing the droplets of the aqueous monomer phase in the organic hydrophobic liquid before and in course of the polymerization or processing of the LP composition. The term “stabilizing” means in the usual manner that the agents prevent the dispersion from aggregation and flocculation.

The process stabilizing agents can be any stabilizing agents, including surfactants, which aim at such stabilization. In certain embodiments, the process stabilizing agents can be oligomeric or polymeric surfactants. Due to the fact that oligomeric and polymeric surfactants can have many anchor groups they absorb very strongly on the surface of the particles and furthermore oligomers/polymers are capable of forming a dense steric barrier on the surface of the particles which prevents aggregation. The number average molecular weight Mn of such oligomeric or polymeric surfactants may for example range from 500 to 60,000 g/mol (e.g., from 500 to 10,000 g/mol, or from 1,000 to 5,000 g/mol). Suitable oligomeric and/or polymeric surfactants for stabilizing polymer dispersions are known to the skilled artisan. Examples of such stabilizing polymers comprise amphiphilic block copolymers, comprising hydrophilic and hydrophobic blocks, amphiphilic copolymers comprising hydrophobic and hydrophilic monomers and amphiphilic comb polymers comprising a hydrophobic main chain and hydrophilic side chains or alternatively a hydrophilic main chain and hydrophobic side chains.

Examples of amphiphilic block copolymers comprise block copolymers comprising a hydrophobic block comprising alkylacrylates having longer alkyl chains, e.g., C₆ to C₂₂-alkyl chains, such as for instance hexyl(meth)acrylate, 2-ethylhexyl(meth)acrylate, octyl(meth)acrylate, do-decyl(meth)acrylate, hexadecyl(meth)acrylate or octadecyl(meth)acrylate. The hydrophilic block may comprise hydrophilic monomers such as acrylic acid, methacrylic acid or vinyl pyrrolidone.

### Inverted Polymer Solutions

Also provided herein are inverted polymer solutions, as well as methods of preparing the inverted polymer solutions from the polymer compositions described herein and methods for using the inverted polymer solutions in oil and gas operations.

Methods for preparing inverted polymer solutions from the polymer compositions described herein can comprise inverting the polymer composition in an aqueous fluid to provide an inverted polymer solution having a concentration of one or more synthetic (co)polymers (e.g., one or more acrylamide (co)polymers) of from 50 to 50,000 ppm (e.g., 50 to 15,000 ppm).

In some embodiments, the inverted polymer solution can have a concentration of one or more synthetic (co)polymers (e.g., one or more acrylamide (co)polymers) of at least 50 ppm (e.g., at least 100 ppm, at least 250 ppm, at least 500 ppm, at least 750 ppm, at least 1000 ppm, at least 1500 ppm, at least 2000 ppm, at least 2500 ppm, at least 3000 ppm, at least 3500 ppm, at least 4000 ppm, at least 4500 ppm, at least 5000 ppm, at least 5500 ppm, at least 6000 ppm, at least 6500 ppm, at least 7000 ppm, at least 7500 ppm, at least 8000 ppm, at least 8500 ppm, at least 9000 ppm, at least 9500 ppm, at least 10,000 ppm, at least 10,500 ppm, at least 11,000 ppm, at least 11,500 ppm, at least 12,000 ppm, at least 12,500 ppm, at least 13,000 ppm, at least 13,500 ppm, at least 14,000 ppm, at least 14,500 ppm, at least 15,000 ppm, at least 20,000 ppm, at least 25,000 ppm, at least 30,000 ppm, at least 35,000 ppm, at least 40,000 ppm, or at least 45,000 ppm).

In some embodiments, the inverted polymer solution can have a concentration of one or more synthetic (co)polymers (e.g., one or more acrylamide (co)polymers) of 50,000 ppm or less (e.g., 45,000 ppm or less, 40,000 ppm or less, 35,000 ppm or less, 30,000 ppm or less, 25,000 ppm or less, 20,000 ppm or less, 15,000 ppm or less, 14,500 ppm or less, 14,000 ppm or less, 13,500 ppm or less, 13,000 ppm or less, 12,500 ppm or less, 12,000 ppm or less, 11,500 ppm or less, 11,000 ppm or less, 10,500 ppm or less, 10,000 ppm or less, 9,500 ppm or less, 9,000 ppm or less, 8,500 ppm or less, 8,000 ppm or less, 7,500 ppm or less, 7,000 ppm or less, 6,500 ppm or less, 6,000 ppm or less, 5,500 ppm or less, 5,000 ppm or less, 4500 ppm or less, 4000 ppm or less, 3500 ppm or less, 3000 ppm or less, 2500 ppm or less, 2000 ppm or less, 1500 ppm or less, 1000 ppm or less, 750 ppm or less, 500 ppm or less, 250 ppm or less, or 100 ppm or less).

The inverted polymer solution can have a concentration of one or more synthetic (co)polymers (e.g., one or more acrylamide (co)polymers) ranging from any of the minimum values described above to any of the maximum values described above. For example, in some embodiments, the inverted polymer solution can have a concentration of one or more synthetic (co)polymers (e.g., one or more acrylamide (co)polymers) of from 500 to 15,000 ppm (e.g., from 500 to 5000 ppm, from 500 to 3000 ppm, or from 500 to 1500 ppm).

In some embodiments, the inverted polymer solution can be an aqueous unstable colloidal suspension. In other embodiments, the inverted polymer solution can be an aqueous stable solution.

In some embodiments, when the LP composition is inverted in an aqueous fluid, providing an inverted polymer solution having from 50 to 15,000 ppm, from 500 to 5,000 ppm, or from 500 to 3000 ppm, active polymer, the inverted polymer solution has a viscosity of at least 20 cP at 40° C., and a filter ratio (FR) (1.2 micron filter) of 1.5 or less. In certain embodiments, when the LP composition is inverted in an aqueous fluid, providing an inverted polymer solution having from 50 to 15,000 ppm, from 500 to 5000 ppm, or from 500 to 3000 ppm, active polymer, the inverted polymer solution has a viscosity of at least 20 cP at 30° C., and a filter ratio (FR) (1.2 micron filter) of 1.5 or less. As used herein, “inverted” refers to the point at which the viscosity of the inverted polymer solution has substantially reached a consistent viscosity. In practice, this may be determined for example by measuring viscosity of the inverted polymer solution periodically over time and when three consecutive measurements are within the standard of error for the measurement, then the composition is considered inverted. In some embodiments, inversion of the LP forms the inverted polymer solution in 30 minutes or less (e.g., 15 minutes or less, 10 minutes or less, 5 minutes or less, or less).

### Methods

In another aspect, a method of displacing a hydrocarbon material in contact with a solid material is provided. The method includes contacting a hydrocarbon material with a compound as described herein, wherein the hydrocarbon material is in contact with a solid material. The hydrocarbon material is allowed to separate from the solid material thereby displacing the hydrocarbon material in contact with the solid material.

In other embodiments, the hydrocarbon material is unrefined petroleum (e.g., in a petroleum reservoir). In some further embodiments, the unrefined petroleum is an unrefined petroleum with an API gravity greater than 30. In some embodiments, the API gravity of the unrefined petroleum is greater than 30. In other embodiments, the API gravity of the unrefined petroleum is greater than 40. In some embodiments, the API gravity of the unrefined petroleum is greater than 50. In other embodiments, the API gravity of the unrefined petroleum is greater than 60. In some embodiments, the API gravity of the unrefined petroleum is greater than 70. In other embodiments, the API gravity of the unrefined petroleum is greater than 80. In some embodiments, the API gravity of the unrefined petroleum is greater than 90. In other embodiments, the API gravity of the unrefined petroleum is greater than 100. In some other embodiments, the API gravity of the unrefined petroleum is between 30 and 100.

The solid material may be a natural solid material (i.e., a solid found in nature such as rock). The natural solid material may be found in a petroleum reservoir. In some embodiments, the method is an enhanced oil recovery method. Enhanced oil recovery methods are well known in the art. A general treatise on enhanced oil recovery methods is Basic Concepts in Enhanced Oil Recovery Processes edited by M. Baviere (published for SCI by Elsevier Applied Science, London and New York, 1991). For example, in an enhanced oil recovery method, the displacing of the unrefined petroleum in contact with the solid material is accomplished by contacting the unrefined with a compound provided herein, wherein the unrefined petroleum is in contact with the solid material. The unrefined petroleum may be in an oil reservoir. The compound or composition provided herein can be pumped into the reservoir in accordance with known enhanced oil recovery parameters. The compound can be pumped into the reservoir as part of the aqueous compositions provided herein and, upon contacting the unrefined petroleum, form an emulsion composition provided herein.

In some embodiments, the natural solid material can be rock or regolith. The natural solid material can be a geological formation such as elastics or carbonates. The natural solid material can be either consolidated or unconsolidated material or mixtures thereof. The hydrocarbon material may be trapped or confined by “bedrock” above or below the natural solid material. The hydrocarbon material may be found in fractured bedrock or porous natural solid material. In other embodiments, the regolith is soil.

In some embodiments, an emulsion forms after the contacting step. The emulsion thus formed can be the emulsion described above. In some embodiments, the emulsion thus formed can be a microemulsion. In some embodiments, the method includes allowing an unrefined petroleum acid within the unrefined petroleum material to enter into the emulsion, thereby converting the unrefined petroleum acid into a surfactant. In other words, where the unrefined petroleum acid converts into a surfactant it is mobilized and therefore separates from the solid material.

In another aspect, a method of converting (e.g., mobilizing) an unrefined petroleum acid into a surfactant is provided. The method includes contacting a petroleum material with an aqueous composition thereby forming an emulsion in contact with the petroleum material, wherein the aqueous composition includes the compound described herein. Thus, in some embodiments, the aqueous composition is the aqueous composition described above. An unrefined petroleum acid within the unrefined petroleum material is allowed to enter into the emulsion, thereby converting the unrefined petroleum acid into a surfactant. In some embodiments, the reactive petroleum material is in a petroleum reservoir. In some embodiments, as described above and as is generally known in the art, the unrefined petroleum acid is a naphthenic acid. In some embodiments, as described above and as is generally known in the art, the unrefined petroleum acid is a mixture of naphthenic acid. In some embodiments, the aqueous composition further includes an alkali agent.

In these embodiments, the composition can comprise a compound described herein, an alkali agent, an additional surfactant, a co-solvent, and a polymer. Methods can comprise injecting a composition of this type into a hydrocarbon reservoir comprising unrefined petroleum material in contact with the solid material. In certain embodiments, the unrefined petroleum material can comprise an active oil. In these embodiments, the composition can have a pH effective to convert unrefined petroleum acid present in the unrefined petroleum material into a surfactant.

In these embodiments, the composition can comprise a compound described herein, an alkali agent, co-solvent, and a polymer. Methods can comprise injecting a composition of this type into a hydrocarbon reservoir comprising unrefined petroleum material in contact with the solid material. In certain embodiments, the unrefined petroleum material can comprise an active oil. In these embodiments, the composition can have a pH effective to convert unrefined petroleum acid present in the unrefined petroleum material into a surfactant.

In these embodiments, the composition can comprise a compound described herein, a co-solvent, and a polymer. Methods can comprise injecting a composition of this type into a hydrocarbon reservoir comprising unrefined petroleum material in contact with the solid material.

In these embodiments, the composition can comprise a compound described herein. Methods can comprise injecting a composition of this type into a hydrocarbon reservoir comprising unrefined petroleum material in contact with the solid material. In certain embodiments, the unrefined petroleum material can comprise an active oil. In these embodiments, the composition can have a pH effective to convert unrefined petroleum acid present in the unrefined petroleum material into a surfactant.

In another aspect, a method of making a compound as described herein is provided. The methods can include contacting a suitable alcohol precursor for the surfactant (e.g., a polyol) with a propylene oxide thereby forming a polyol alkoxylate hydrophobe.

In some aspect, described herein are also methods for preparing an inverted polymer solution comprising providing a liquid polymer (LP) composition comprising: one or more hydrophobic liquids, one or more synthetic (co)polymers, an inversion package comprising a compound described herein, and optionally one or more surfactants.

As described above, methods for preparing an inverted polymer solution from the LP composition described herein can comprise inverting the LP composition in an aqueous fluid to provide an inverted polymer solution having a concentration of acrylamide (co)polymer of from 50 to 15,000 ppm.

The LP compositions described herein can be inverted using inversion methods and systems known in the art, such as those described in U.S. Pat. Nos. 8,383,560, and 10,626,320 which is hereby incorporated by reference in its entirety.

For example, inversion of the LP composition can be performed as a batch process or a continuous process. In certain embodiments, inversion of the LP composition can be performed as a continuous process. For example, inversion of the LP composition can be performed as a continuous process to produce a fluid stream for injection into a hydrocarbon-bearing formation. A continuous process is a process that can be effected without the need to be intermittently stopped or slowed. For example, continuous processes can meet one or more of the following criteria: (a) materials for forming the inverted polymer solution (e.g., the LP composition and the aqueous fluid) are fed into the system in which the inverted polymer solution is produced at the same rate as the inverted polymer solution is removed from the system; (b) the nature of the composition(s) introduced to the system in which the inverted polymer solution is produced is a function of the composition(s) position with the process as it flows from the point at which the composition(s) are introduced to the system to the point at which the inverted polymer solution is removed from the system; and/or (c) the quantity of inverted polymer solution produced is a function of (i) the duration for which the process is operated and (ii) the throughput rate of the process.

Inversion of the LP composition can comprise a single step, or a plurality of steps (i.e., two or more steps). In some embodiments, inversion of the LP composition can be performed in a single step. In these embodiments, the LP composition (e.g., a composition having at least 39% (e.g., 39% or more) by weight of one or more synthetic (co)polymers (e.g., one or more acrylamide (co)polymers) dispersed in a hydrophobic liquid, or a composition having up to 35% (e.g., less than 35%) by weight of one or more synthetic (co)polymers (e.g., one or more acrylamide (co)polymers) emulsified in a hydrophobic liquid) can be inverted in an aqueous fluid to provide an inverted polymer solution having a concentration of one or more synthetic (co)polymers (e.g., one or more acrylamide (co)polymers) of from 50 to 15,000 ppm.

By way of non-limiting illustration, examples of certain embodiments of the present disclosure are given below.

### EXAMPLES

The examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Alkali-Co-Solvent-Polymer (ACP) Using Surface Active Solvent/Co-Solvent (SAS) for Heavy Oils

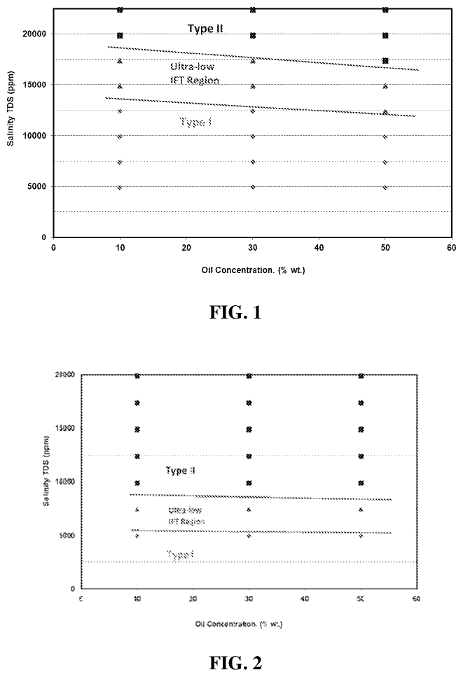

ACP refers to Alkali-Co-solvent-Polymer enhanced oil recovery process applied to heavy oils which are termed “Active”. Active Oils under alkaline conditions generate natural soaps. No additional surfactant is needed. These natural soaps are, in general, hydrophobic. Hydrophilic co-solvents are needed for good Phase Behavior. When the co-solvent has some surface activity (SAS), in addition to hydrophilicity, better Phase Behavior is expected from the system. The co-solvent needs to be liquid at least around ˜25° C., to produce Ultra-Low (UL)-IFT with lowest possible concentration, and have an acceptable optimum salinity with Na₂CO₃. As demonstrated in FIG. 1 increasing EO's increases the optimum salinity, however, the solvent become solid at room temperature.

Polyol propoxylates show promise as surface-active solvents/Co-solvents (SAS). SAS (1% Glycerine-XPO (Tri Hydroxy Propoxylate-No EO) exhibit UL-IFT phase behavior with the heavy oil tested at a lower temperature under alkaline pH. Optimum salinity did not change significantly with the SAS concentration. Lower concentrations of SAS also show UL-IFT in PB (phase behavior) experiments.

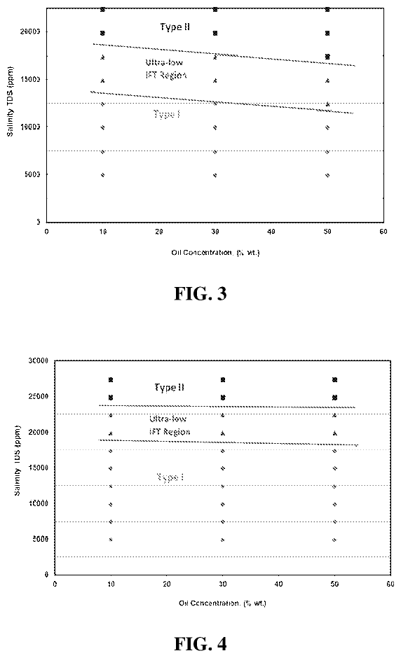

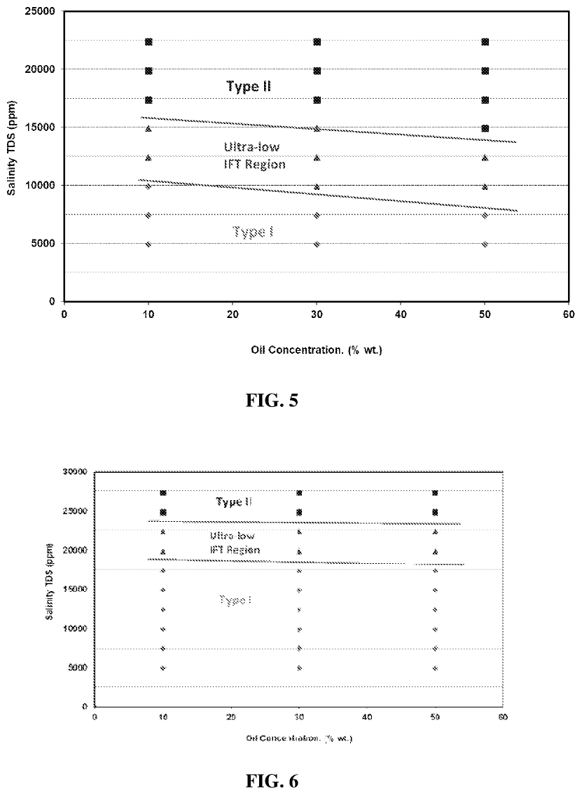

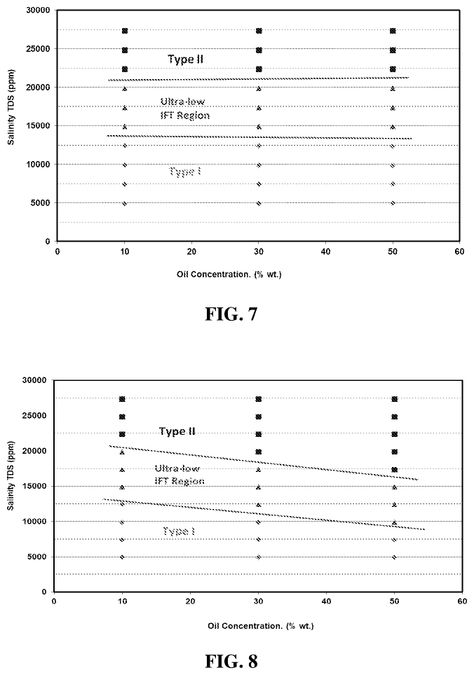

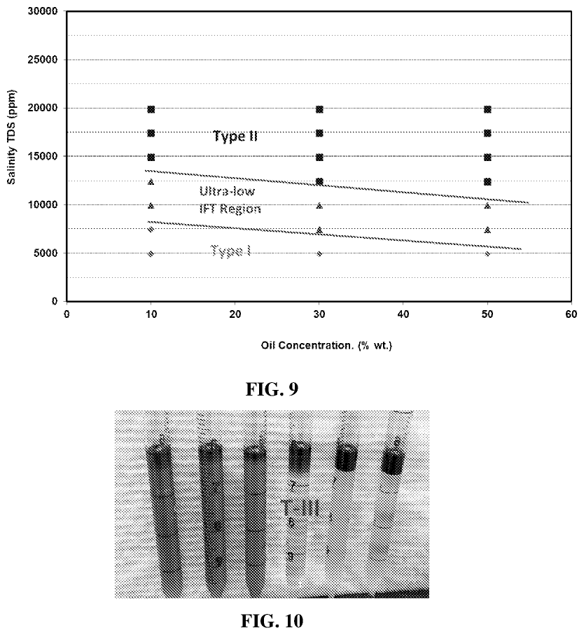

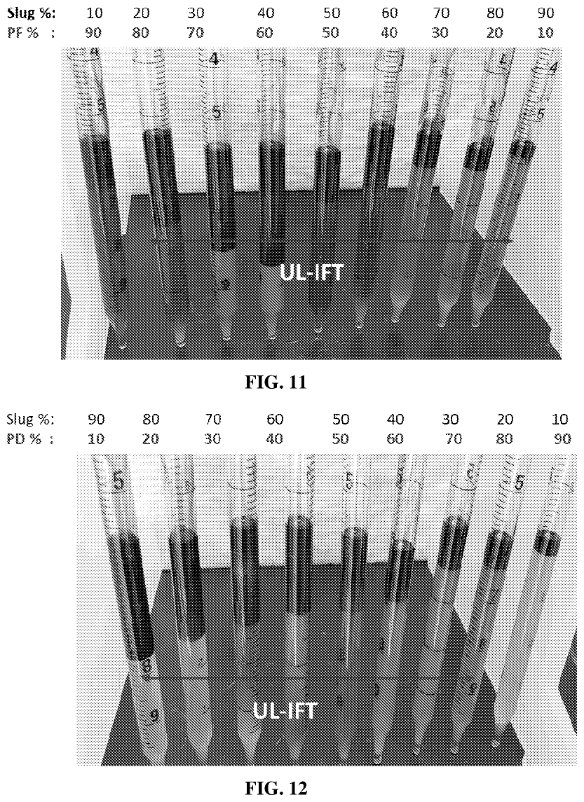

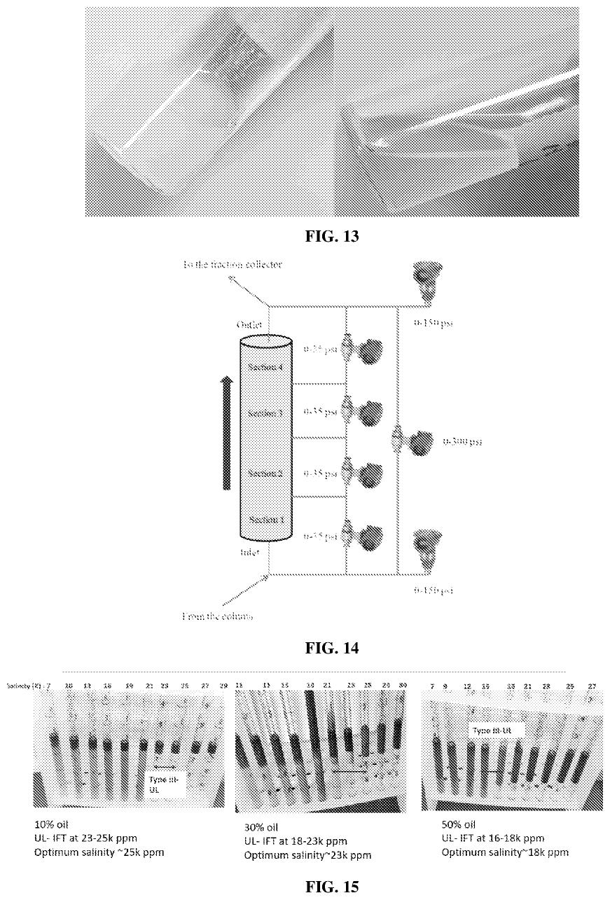

For example, as shown in FIGS. 2-4 increasing the PO groups increases surface activity and optimum salinity while maintaining aqueous stability at room temperature or lower temperature. When using 0.25% glycerine-6PO optimum salinity was lower than 0.25% Na₂CO₃. FIG. 5 shows optimum salinity when using 0.25% glycerine-12PO. As shown in FIGS. 6-9 glycerine-25PO shows to be more tolerant to change in optimum salinity when lowering the concentration of the co-solvent. All of the co-solvent exhibited UL-IFT phase behavior even with lower co-solvent concentration.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

### Example 2: Implementing Alkali-Surfactant-Polymer/Alkali-Cosolvent-Polymer (ASP/ACP) In Difficult Environments—Evaluation with High TAN Viscous Crude Oil

Research on alkali assisted chemical EOR technology (alkali-surfactant-polymer/alkali-cosolvent-polymer) with high TAN crude oils have led to developments with liquid organic alkalis and co-solvents (Southwick J., et al., 2020; Fortenberry, et al., 2015; Schumi, et al., 2019; Upamali, et al., 2018). Both concepts afford potential significant cost reduction in field operations but to date it has not been demonstrated that these two concepts can work together.

Monoethanolamine (MEA) alkali and a wide variety of liquid co-solvents are evaluated with high TAN (total acid number) crude oil (1.5 mg KOH/g oil). Formulations are found that give ultra-low interfacial tension (IFT) at a specified injection salinity. Fine tuning the formulation to different injection salinities can be done by choosing alternate co-solvents (or a co-solvent blend). A formulation comprising 1% MEA and a novel high molecular weight (3,152 g/gmol) co-solvent, 0.5% glycerin alkoxylate with 30 moles propylene-oxide and 35 moles ethylene-oxide (glycerin-30PO-35EO), gave ultra-low IFT in 21,000 TDS injection brine and gave 100% oil recovery in Bentheimer sandstone with 3,500 ppm FP 3630s (SNF Flopaam 3630s partially hydrolyzed polyacrylamide) as mobility control agent. All oil was produced clean, no separation of emulsion was needed to measure oil recovery.

Alkali consumption tests were also performed with a high permeability reservoir sandstone. Results confirmed earlier data published with Boise outcrop sandstone (Southwick J., et al., 2020) showing low alkali consumption with MEA. On a mass basis, only 12% of the amount of MEA is consumed relative to the amount of sodium carbonate consumed. This reduces the logistical challenges of shipping chemicals to remote locations. MEA is also a low viscosity liquid which further simplifies field handling.

### Introduction

There has been much research on chemical flooding since the 1960's and hundreds if not more than a thousand publications. Surfactant-polymer, and alkali-surfactant-polymer flooding has been effectively demonstrated in the laboratory (Hirasaki, Miller, & Puerto, 2011; Jang S. H., et al., 2016), with very high ultimate recoveries, and low chemical retention. New surfactants and polymers have been developed for application at high temperature and in high salinity brines (Levitt, et al., 2009; Liu, et al., 2008; Lu, et al., 2014; Gaillard, et al., 2015; Gaillard, et al., 2021), The importance of recognizing the difference between live and dead crude on surfactant behavior has been studied and specific procedures developed to assure formulations are effective with live reservoir crude (Nelson R. C., 1983; Southwick, Svec, Chilek, & Shahin, 2012; Jang S., et al., 2014). The developed technology is robust and well-designed laboratory floods typically reduce oil saturation in the core to less than 5%. The critical challenge to transfer laboratory success to field implementation is meeting economic hurdles. In the competition for project capital chemical EOR projects are disadvantaged by high chemical, facility, and logistical cost, and also the need to infill drill to produce a pattern with a short enough well spacing so that increased oil production occurs in a reasonable time frame. A significant development, alkali-surfactant-polymer (ASP) flooding (Nelson, Lawson, Thigpen, & Stegemeier, 1984) reduced the cost of chemical EOR formulations by utilizing naturally occurring petroleum soaps from high TAN crude oils along with hydrophilic “cosurfactant”. These formulations used much less surfactant than surfactant-polymer (SP) formulations and the alkali addition significantly lowered surfactant adsorption on reservoir rock.

Nearly all the published literature is regarding either surfactants, polymers, reservoir engineering aspects, or reports of field operations. Relatively few papers have been published relating to the selection of alkali for ASP flooding, or the logistical challenges of implementing full scale commercial floods in remote locations. It has generally been thought that alkali is a benign component of the formulation, and that surfactants (which lower IFT), and polymers (which increase viscosity), are the more critical components of a formulation. This is true in the laboratory, but once field projects are considered the importance of alkali selection can become critical. This is because for a typical ASP and alkali-cosolvent-polymer (ASP/ACP) formulation (ca. 2.00% sodium carbonate, 0.30% surfactant, 0.25% polymer), alkali comprises 75% of the mass of the formulation. While it is the least expensive component, shipment of such a large quantity of chemical to remote locations can provide logistical issues to the extent that a full-scale field project is not practical.

For this reason, research on using liquid ammonia as alkali was performed and reported (Southwick, et al., 2016). Liquid ammonia has the advantage of having a low molecular weight so that 17 g of ammonia yields one mole of alkalinity. Sodium carbonate by comparison has a molecular weight of 106 g/mole, and one mole of alkali requires 106 g. From this logic 6.2 times less ammonia on a weight basis is necessary for the same equivalent moles of alkali as is required for sodium carbonate. This significantly reduces the amount of chemical needed to be transported for an ammonia ASP flood. Detailed results are reported (Southwick, et al., 2016) and the conclusion reached was that ammonia is a suitable replacement for sodium carbonate. However, safety issues with using relatively large amounts of ammonia in the oil field have delayed implementation of this concept.

Given the safety issues present with liquid ammonia further investigations on alkali focused on organic amines that have the same alkaline characteristics as ammonia, but are liquids at room temperature and pressure, and have lower toxicity. Monoethanolamine (MEA) is such an organic amine. Organic amines were previously evaluated, (Berger & Lee, 2006), and a recent publication (Southwick J., et al., 2020) focused on the performance of MEA in ASP formulations. Positive results were obtained. MEA is a lower molecular weight alkali than sodium carbonate, and surprisingly it was found that alkali consumption with MEA was lower than sodium carbonate (on a molar basis). Moreover, MEA can function as a solvent for specific surfactants raising the possibility that a liquid alkali-surfactant blend (in the correct formulated ratio) can be shipped to the field and injected directly into a soft water polymer stream. With this concept, minimal facilities are needed to implement ASP.

This paper extends the concepts described in earlier work (Southwick J., et al., 2020; Southwick, et al., 2016) to the use of MEA with co-solvent rather than surfactant. Co-solvents (Fortenberry, et al., 2015; Schumi, et al., 2019; Upamali, et al., 2018) can also be effective in reducing IFT with certain oil and reservoir conditions (principally injection brine salinity) and have the advantage relative to surfactant of low retention. They are also low viscosity liquids, which can be easily handled in the field, and potentially blended with MEA.

### Experimental

### Materials Surfactants and Co-Solvents.

Surfactants used in chemical EOR projects are typically supplied as high viscosity concentrates in water. These “pasty” materials require either specialized equipment for field handling, or significant dilution with water to attain lower viscosity, handleable fluids.

Handling such materials in arctic environments is challenging. A description of the challenges faced at the Salym field using sulfonate surfactants in Siberia, Russia has been published (Karpan, et al., 2014).

Another approach is to use co-solvents and/or liquid surfactants. In environments that experience subfreezing temperatures it is also critical that the liquid blends remain free flowing at low temperature. A significant advantage of these products is that many of the chemicals are soluble in MEA. Therefore, mixtures of MEA and co-solvent/surfactants were studied to identify those that remained liquid at low temperatures while remaining viable solutions for ACP/ASP. Error! Reference source not found. below shows experiments performed with mixtures of MEA, alkoxylates and co-solvents. Some of the blends have suitable low temperature properties. SBA, IBA, and NBA are sec, iso, and normal butyl alcohol, respectively. DIPA is diisopropylamine, 2EH is 2-ethylhexanol, EGBE is ethylene glycol mono-butyl ether, PO is propylene oxide, EQ is ethylene oxide. The alkoxylates are 100% liquid products, and further product development in this area can potentially tailor-make a product for a specific reservoir condition. It is seen in Error! Reference source not found. below that several of the blends are liquid at −20° C.

TABLE 1
Properties of concentrated mixtures

| Alkoxylate, | | wt % Co- | 22° C. | 8° C. | −20° C. |
|---|---|---|---|---|---|
| wt % | MEA | Solvent | Comments | Comments | Comments |
| 2EH-15PO- | 50% | 25% EGBE | Solid at | Not | Not |
| 30EO, 25% | | | bottom | Tested | Tested |
| 2EH-15PO- | 50% | 25% IBA- | Solid at | Not | Not |
| 30EO, 25% | | 1PO-2EO | bottom | Tested | Tested |
| 2EH-15PO- | 35% | 40% EGBE | Solid at | Not | Not |
| 30EO, 25% | | | bottom | Tested | Tested |
| 2EH-15PO- | 35% | 40% IBA- | Solid at | Not | Not |
| 30EO, 25% | | 1PO-2EO | bottom | Tested | Tested |
| 2EH-15PO- | 50% | 25% NBA | Solid at | Not | Not |
| 30EO, 25% | | | bottom | Tested | Tested |
| 2EH-15PO- | 50% | 25% IBA | Solid at | Not | Not |
| 30EO, 25% | | | bottom | Tested | Tested |
| 2EH-15PO- | 50% | 25% SBA | Solid at | Not | Not |
| 30EO, 25% | | | bottom | Tested | Tested |
| 2EH-15PO- | 50% | 25% SBA | Liquid | Liquid | Viscous |
| 10EO, 25% | | | | | liquid |
| 2EH-7PO- | 50% | 25% SBA | Liquid | Liquid | Viscous |
| 10EO, 25% | | | | | liquid |
| CH3-15PO- | 40% | 35% SBA | Solid at | Precip- | Solid |
| 30EO, 25% | | | bottom | itation | |
| 2EH-15PO- | 50% | 35% SBA | Solid at | Precip- | Solid |
| 30EO, 15% | | | bottom | itation | |
| 2EH-15PO- | 50% | 25% NBA | Phase | Liquid | Viscous |
| 10EO, 25% | | | separation | | liquid |
| 2EH-07PO- | 50% | 25% NBA | Liquid | Liquid | Viscous |
| 10EO, 25% | | | | | liquid |
| DIPA-7PO- | 50% | 25% NBA | Liquid | Liquid | Viscous |
| 10EO, 25% | | | | | liquid |
| CH3-40PO- | 50% | 25% NBA | Liquid | Liquid | Viscous |
| 10EO, 25% | | | | | liquid |
| CH3-21PO, | 50% | 25% NBA | Liquid | Liquid | Viscous |
| 25% | | | | | liquid |
| CH3-15PO- | 50% | 25% NBA | Liquid | Precip- | Solid |
| 20EO, 25% | | | | itation | |
| CH3-70PO- | 50% | 25% NBA | Solid at | Precip- | Solid |
| 100EO, 25% | | | bottom | itation | |
| 2EH-15PO- | 50% | 25% NBA | Solid at | Precip- | Solid |
| 60EO, 25% | | | bottom | itation | |
| 2EH-15PO- | 50% | 12.5% IBA- | Phase | Liquid | Viscous |
| 10EO, 37.5% | | 1PO-2EO | separation | | liquid |
| 2EH-15PO- | 50% | 20% IBA- | Liquid | Liquid | Viscous |
| 10EO, 30% | | 1PO-5EO | | | liquid |
| 2EH -15PO- | 50% | 25% IBA- | Liquid | Slightly | Solid |
| 10EO, 25% | | 1PO-5EO | | hazy | |
| 2EH -15PO- | 50% | 30% IBA- | Clear | Slightly | Viscous |
| 10EO, 20% | | 1PO-5EO | liquid | hazy | liquid |
| 2EH -15PO- | 50% | 30% SBA | Clear | Clear | Viscous |
| 10EO, 20% | | | liquid | liquid | liquid |
| 2EH -15PO- | 50% | 35% NBA | Solid at | Not | Not |
| 10EO, 15% | | | bottom | Tested | Tested |
| 2EH -15PO- | 50% | 35% SBA | Solid at | Not | Not |
| 10EO, 15% | | | bottom | Tested | Tested |
| CH3-15PO- | 50% | 30% NBA | Solid at | Not | Not |
| 20EO, 20% | | | bottom | Tested | Tested |
| 2EH-15PO- | 50% | 15% IBA- | Phase | Not | Not |
| 10EO, 35% | | 1PO-5EO | separation | Tested | Tested |
| 2EH-15PO- | 50% | 30% EGBE | Phase | Not | Not |
| 10EO, 20% | | | separation | Tested | Tested |
| 2EH-15PO- | 50% | 20% | Phase | Not | Not |
| 10EO, 30% | | Phenol- | separation | Tested | Tested |
| | | 1PO-10EO | | | |
| 2EH-15PO- | 50% | 25% | Liquid | Clear | Semi-solid |
| 10EO, 25% | | Phenol- | | liquid | |
| | | 1PO-5EO | | | |
| 2EH-15PO- | 50% | 25% | Liquid | Clear | Liquid |
| 10EO, 25% | | Phenol-2EO | | liquid | |
| Phenol-1PO- | 50% | 10% NBA | Liquid | Liquid | Solid |
| 10EO, 40% | | | | | |
| Phenol-10EO, | 50% | 10% NBA | Liquid | Liquid | Solid |
| 40% | | | | | |
| Phenol-1PO- | 50% | 20% NBA | Liquid | Liquid | Solid |
| 10EO, 30% | | | | | |
| Phenol-10EO, | 50% | 15% NBA | Liquid | Liquid | Solid |
| 35% | | | | | |
| Phenol-10EO, | 50% | 25% NBA | Liquid | Liquid | Solid |
| 25% | | | | | |
| Phenol-4EO, | 50% | 15% NBA | Liquid | Liquid | Liquid |
| 35% | | | | | |
| Phenol-1PO- | 50% | — | Liquid | Liquid | Liquid |
| 5EO, 50% | | | | | |

### Crude Oil Properties

Oil properties are shown in Table 2. The total acid number (TAN; mg KOH/g oil) is high (1.5), and the oil is a suitable candidate for ASP or ACP flooding. This is confirmed by phase behavior results which showed significant IFT reduction between brine and oil with increase in pH.

TABLE 2
Crude Oil Properties

| | | Surrogate Oil | Total Acid |
|---|---|---|---|
| | Reservoir Temp. | Viscosity | Number |
| Dead Oil | [° C.] | @ 22° C. [cP] | mg/g |
| Crude A | 22 | 60 | 1.5 |

### Brine Compositions

Compositions of softened (SIW) and hard injection water (IW), connate water (CW), and a higher salinity softened supply brine (SSB) source are shown in Table 2 and FIG. 22. The most important characteristics relevant to surfactant behavior (partitioning between brine and oil phases) is the total dissolved solids (TDS) and the divalent ion concentration. Softened brine is necessary to avoid precipitation of carbonate by divalent cations which is necessary to achieve good injectivity. This is not as severe an issue with MEA as it is with sodium carbonate, but carbonate anion is also formed from bicarbonate (very often present in source brines) at pH values above 8.5.

TABLE 2
Synthetic Brines for Experiments

| | | |
|---|---|---|
| Synthetic Injection Water (IW) | 2,800 | 130 |
| Synthetic Softened Injection Water | 2,470 | 0 |
| (SIW) | | |
| Synthetic Connate Brine (CB) | 23,400 | 400 |
| Synthetic Softened Supply Brine | 21,600 | 0 |
| (SSB) | | |

### Methods

### Phase Behavior—Surfactant Screening, Solubility Parameter, Interfacial Tension

It is important to establish an efficient procedure for evaluating the capability of co-solvents and surfactants to form low interfacial tension in alkaline solutions with crude oil. There exist a wide variety of products to be considered and these products can also be used together in blends of varying ratio. Furthermore, the evaluation of formulations is more complicated when co-solvents and surfactants are used to form low IFT in conjunction with petroleum soaps formed by saponification of crude oil with alkali (ACP, ASP) than when surfactants are used alone (SP flooding). This is because the ratio of petroleum soap to co-solvent/surfactant is important, and the relative ratio of these components at the brine oil interface is a function of oil saturation (Nelson, Lawson, Thigpen, & Stegemeier, 1984).

A series of burettes are prepared for a range of salinities bracketing the connate water salinity at 10%, 30%, and 50% oil. The brine oil mixtures are vigorously mixed and then equilibrated at reservoir temperature for one week. The burettes are evaluated, and it is determined if the resulting Winsor emulsion type is either oil in brine (type I), brine in oil (type II), or whether a third phase has formed (type III) (Winsor, 1954). Type III is desirable as this is the condition of lowest IFT. It is desirable if the type III range of salinity is not strongly dependent on oil/brine ratio (but this is often not the case). This brings the question of which oil concentration to use for determination of optimal salinity—the condition of minimum IFT. For a stable displacement with similar conditions, it was argued that 30% oil is a good assumption (Southwick J. G., Brewer, Pieterse, & Batenburg, 2018).

### Coreflood Procedures

A Bentheimer core was loaded in a 1 ft core holder with three pressure taps. A schematic of the coreflood setup is shown in FIG. 14. A confining pressure of 1,000 psi was applied to the core.

The core was slowly flooded with many pore volumes of the reducing solution (1% Na₄EDTA, 4% KHCO₃, 1% sodium dithionite) until effluent ORP was <−400 mV and iron content <3 ppm.

The core was then flooded with 1% KCl+500 ppm sodium dithionite (NaDT) and pressure drop was monitored.

A pore volume tracer test was performed using 5% KCl containing 500 ppm sodium dithionite at 86 ft/day (5 ml/min), 5-7 ml fractions were collected.

Core was flooded with connate brine at different flow rates for several pore volumes to measure permeability.

After the brine flood, core was flooded with surrogate oil (crude A+20% decane) to match live-oil viscosity and equivalent alkane carbon number (EACN) (Chang, Jang, Tagavifar, & Pope, 2018; Jang S., et al., 2014) at 17.2 ft/day (1 ml/min) and pressure drop was measured see FIG. 30.

An ACP flood was commenced at 0.5 ft/day (0.032 ml/min) after the oil flood. The ACP slug was prepared in SSB brine. Two polymer drives (PD1 & PD2 respectively) were followed the slug, PD1 in a blend of SIW and SSB, roughly 10,000 ppm), and PD2 in hard IW brine. All solutions contained 500 ppm sodium dithionite and 500 ppm diethyl-thiourea. Effluent analysis including viscosity and pH was performed.

### Results

### Phase Behavior Results

Here we show the results of the formulation that gave ultra-low interfacial tension and an advantageous minimal dependence on oil concentration. This is the formulation that was used in the coreflood; 1% MEA and 0.5% glycerin-30PO-35EO. FIG. 15 shows photos of the phase behavior tubes, where optimal salinity, e.g., salinity of lowest IFT Winsor type III, is identified by red arrows. Co-solvents are more hydrophilic than the petroleum soaps formed by saponification of petroleum acids with alkali, so a higher salinity is optimum at low oil concentration.

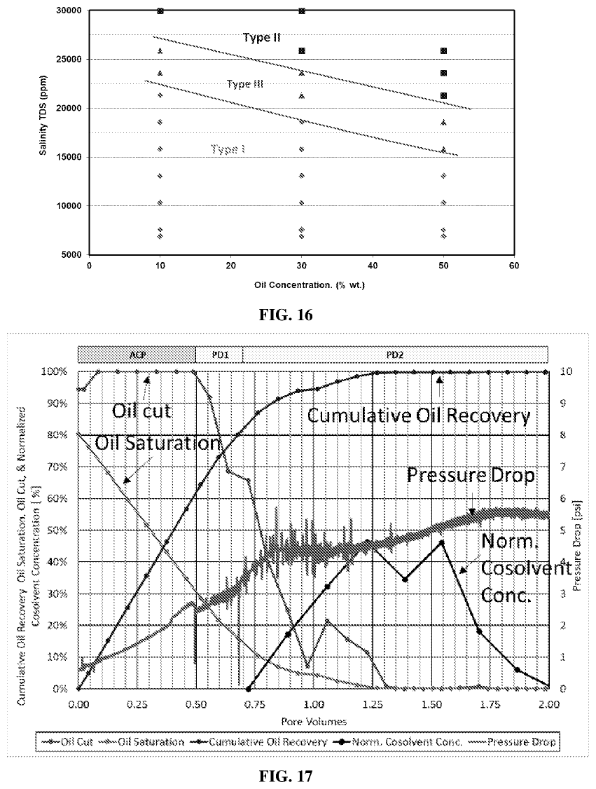

FIG. 16 shows the activity map constructed from the data. The activity map is considerably less steep than typical ASP formulations, which is advantageous. Red arrows indicate the type III region of ultra-low IFT. It is advantageous for a core flood to remain in the type III region as the fluids propagate through the core. As the oil bank increases oil saturation the optimal salinity is reduced. For this reason, it is common practice to inject ca. 0.3 PV of chemical slug at optimal salinity followed by a polymer drive at lower salinity.

It is important to evaluate the consumption of alkali directly with reservoir rock as sufficient alkali must be propagated from injector to producer to maintain low IFT; reservoir rocks vary widely in their consumption of alkali (largely due to variation in type and amount of clay minerals). Previous studies (Southwick J., et al., 2020) argued that MEA alkali is consumed by Boise outcrop sandstone rock to a lesser degree than sodium carbonate alkali due to the lower salinity of the injection brine (sodium carbonate is a salt, whereas MEA is not). This is a surprising result, since alkali consumption has been assumed to be determined by rock properties and not affected by the type of alkali (other than effects of very high pH with NaOH). As these results were reported for Boise sandstone it is critical to test whether or not the same results will be attained with reservoir rock.

Four reservoir core plugs were butted together for experimentation making a total length of 30.2 cm. The core plugs were from a high permeability sandstone.

After the permeability test with 5% KCl, several pore volumes of IW were injected to displace the KCl brine. Then, 0.5% MEA in soft IWA was prepared and injected at 2 ft/D for several pore volumes. Effluent samples were collected, and pH was measured. After the MEA alkali test, several pore volumes of hard brine were injected to displace the MEA. A second alkali test with 2% Na2CO3 in soft brine was prepared and injected for several pore volumes. Effluent samples were collected, and pH was measured.

Alkali consumption for MEA and Na2CO3 was calculated by the amount of injected alkali versus measured recovered alkali. To calculate the recovered alkali, pH measurements of the effluent samples were converted to hydroxide ion equivalent. Cumulative injected alkali at 2.3 pore volumes of continuous injection for each test was 1.9E-02 mol and 4.4E-02 mol for MEA and Na2CO3, respectively. Cumulative recovered alkali for MEA and Na2CO3 was 1.7E-02 mol and 3.3E-02 mol respectively, Table 3. This means that only 12% (153 mg MEA/1230 mg Na2CO3) as much MEA was consumed on a mass basis when compared to Na2CO3.

TABLE 3
MEA & Na2CO3 consumption

| | MEA | Na₂CO₃ |
|---|---|---|
| Injected alkali | 1.9E−02 mol | 4.4E−02 mol |
| Recovered alkali | 1.7E−02 mol | 3.3E−02 mol |
| Alkali consumed | 2.5E−03 mol | 1.2E−02 mol |
| Alkali consumed, mg | 153 | 1230 |

### Oil Recovery Core Flood in Bentheimer Core

### Core Properties and Injected Fluid Compositions

For the oil recovery experiment high permeability Bentheimer sandstone was used. Properties of the core are shown in Table 4.

TABLE 4
Bentheimer core properties

| | |
|---|---|
| Mass (g) | 698 |
| Total Length (cm) | 30.2 |
| Diameter (cm) | 3.8 |
| Cross-sectional Area (cm2) | 11.4 |
| Bulk Volume (mL) | 344.6 |
| Pore Volume (mL) | 82.9 |
| Porosity | 0.24 |
| Permeability | 1900 mD |
| Section 1 length (cm) | 7.62 |
| Section 2 length (cm) | 7.62 |
| Section 3 length (cm) | 7.62 |
| Section 4 length (cm) | 7.37 |

### Brine Flood and Oil Flood

The core was flushed with 100 ppm sodium dithionite solution in connate brine (Table 2) to remove any oxidized iron (Rajapaksha, et al., 2014). A KCl tracer test was performed to remeasure the pore volume of the core. Measured pore volume was 82.9 mL, Table 4. After the KCl tracer test, several more pore volumes of connate brine were injected at different rates to measure brine permeability of the core.

After the brine flood, an oil flood with surrogate oil was injected at 17.2 ft/day (1 mL/min) to establish initial oil saturation of 80%.

Chemical Flood with ACP Design

An ACP slug was prepared in softened supply brine, SSB (Table 2). The ACP slug was injected at a rate of 0.5 ft/D (0.032 ml/min) following oil saturation. As many heavy oil fields are in initial stages of recovery it is appropriate to determine oil recovery starting from a high saturation. The slug was injected at a salinity of ˜21,100 ppm TDS for 0.5 PV before switching to polymer drive 1 (PD1). PD1 contained 3,000 ppm FP 3630s (Flopaam 3630s partially hydrolyzed polyacrylamide) in a soft brine mix (SIW and SSB), roughly 10,000 ppm TDS. PD1 was injected for ˜0.25 pore volume at 0.5 ft/D (0.032 ml/min). PD2 contained 2,500 ppm FP 3630s in IW hard brine. PD2 was injected for another 1.25 pore volume at 0.5 ft/day. Properties of the injected coreflood solutions are shown below in Table 5.

TABLE 5
Properties of the injected coreflood solutions

| | | | Polymer | | Polymer | |
|---|---|---|---|---|---|---|
| Solution Type | ACP Slug | | Drive 1 | | Drive 2 | |
| Pore Volume Injected | 0.50 | | 0.25 | | 1.25 | |
| Polymer: SNF FP 3630s | 3,500 | ppm | 3,000 | ppm | 2,500 | ppm |
| Co-solvent: | 0.5% | | — | | — | |
| glycerin-30PO-35EO | | | | | | |
| Alkali: MEA | 1.00% | | — | | — | |
| Frontal Velocity | ~0.5 | ft/day | ~0.5 | ft/day | ~0.5 | ft/day |
| Total Salinity, TDS | 21,100 | ppm | ~10,000 | ppm | 2,814 | ppm |
| Brine | SSB - soft | | Mix - soft | | IWA - Hard | |
| pH | 10.4 | | 6.7 | | 5.4 | |
| Viscosity at 21° C., | 123 | cP | 118 | cP | 108 | cP |
| 7.3 s−1 | | | | | | |

### Oil Production.

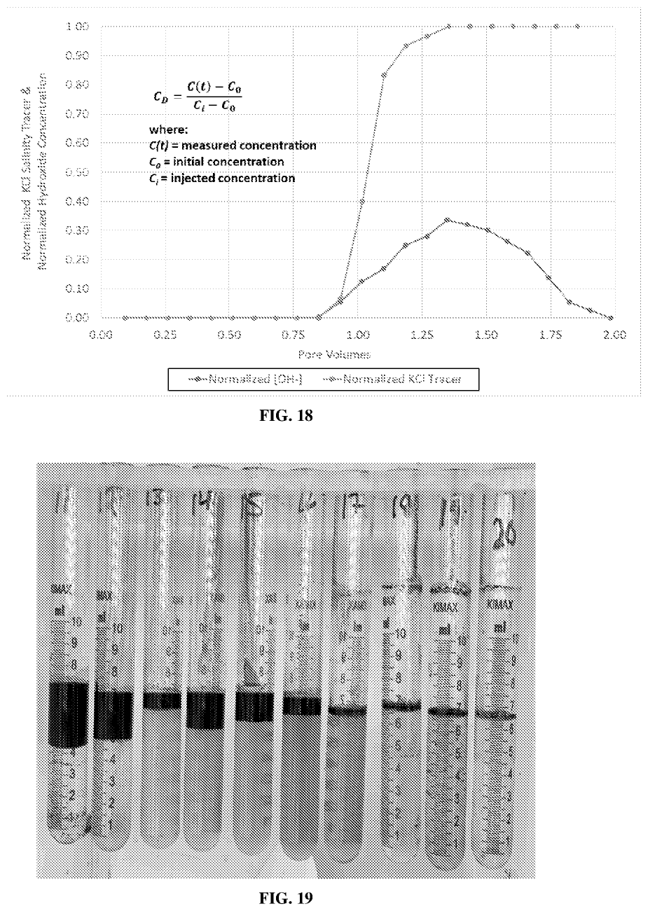

Core flood results are shown in FIG. 17 and FIG. 18. The oil cut was 100% until 0.5 PV produced. Oil production was complete by 1.3 PV injected. A picture of effluent tubes 11-20 (PV 0.8 to 1.6) is shown in FIG. 19. Significantly all the oil is produced as clean oil. This is in contrast to many ASP floods where following production of the main oil bank a large amount of oil in water emulsion is produced. Such emulsions can be problematic in field operations. The polymer drive was split into two parts to protect the ACP slug from mixing with hard brine. Given the excellent results of this flood and the stable displacement this is probably not be necessary. The secondary oil bump beginning around one PV, may be indicative that a more hydrophilic initial polymer drive could be implemented (single polymer drive with IW salinity).

### Chemical Retention.

Effluent samples were collected and produced alkali and co-solvent were measured. Results are shown in Error! Reference source not found. below:

TABLE 6
Chemical consumption during core flood

| Chemical Effluent | Consumption (mg/g rock) |
|---|---|
| Monoethanolamine (MEA) | 0.33 |
| Glycerin-30PO-35EO | 0.12 |

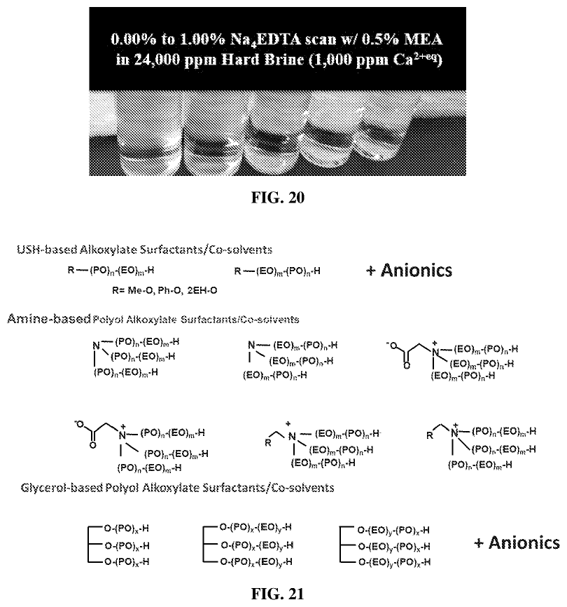

It is important to realize that relative to many other ASP floods reported in the literature these chemical consumption values are low (Jang S. H., et al., 2016). The flood was performed in a Bentheimer sandstone which has low clay content; however, the values are lower than typically observed in core floods performed with viscous oil using sodium carbonate and surfactant. Also, it is possible that a significant part of the co-solvent consumption is due to partitioning into the oil. This implies that co-solvent consumption with reservoir rock may not be significantly higher. The next logical step is to repeat this ACP flood, modify the polymer drive design, and fine tune the concentrations of chemical to achieve optimum economic performance. FIG. 20 below shows that 0.5% MEA is compatible with hard produced connate brine (CB) in Table 2. With PD1 likely not necessary, combined with the lack of precipitation seen in the lab, a polymer drive of hard injection water (IW) may be used This will save on water softening costs and reduce the complexity of using a mix of brines in the field.

### Discussion

The results shown above demonstrate the concept of using a blend of liquid alkali (MEA) and liquid co-solvent (glycerin-30PO-35EO) to generate ultra-low IFT with a high TAN crude oil and recover all the oil in 1.3 PV injection from a Bentheimer outcrop sandstone. This result suggests a lower cost, more efficient chemical oil recovery process than ASP formulations utilizing sodium carbonate with viscous surfactant concentrates in water.

Further work needs to be performed for this approach to be critically evaluated and refined for a field pilot. Oil recovery experiments need to be performed in reservoir rock which are known to normally have higher chemical consumption than Bentheimer sandstone. The coreflood in this study was performed with an excess of polymer which is appropriate to demonstrate proof of concept for ACP with MEA. Injection fluids ranged from 108 to 123 cP whereas the surrogate crude oil viscosity was 60 cP. Chemical retention during the core flood was low. This supports the observations of earlier work where it was shown that alkali consumption (on both a molar and weight basis) is lower for MEA formulations than for Na2CO3. Low co-solvent consumption (Fortenberry, et al., 2015) was also confirmed.

This concept for chemical EOR is appropriate for reservoirs containing acidic crude oils, ca. >0.5 mg KOH/g oil (Qing, 2010), and is most economically attractive with low salinity injection brine. ACP (and ASP) require injection brine without divalent cations (soft brine) to prevent precipitate formation that can plug injection wells. Low salinity allows low-cost water softening, and efficient utilization of HPAM polymer. MEA does not add additional salinity to injection brine (in contrast to Na₂CO₃) reducing the polymer concentration requirement.

A further simplification of ASP implementation in field operations is to avoid water softening for injection brines that contain low levels of divalent cations. The concept is to add scale inhibitor to injection water along with alkali to maintain a small CaCO₃/MgCO₃ precipitate size so that injectivity is not impaired. This is discussed in detail in a recent publication (Farsi, et al., 2020).

Chemical EOR projects in the field have been impacted by complexities regarding produced fluids. Clean oil is most valuable, and if oil needs to be recovered from produced emulsion the economic value is reduced. The core flood described above produced all oil as clean oil with no produced oil in water emulsion. This is a significant result because if this translates to less produced emulsion in the field. Substantially improved economics will occur without emulsion issues.

Alkaline based chemical EOR (ASP) has also been impacted by scale formation in production wells. This has been extensively described in recent literature (Volokitin, et al., 2018; Pandey, Koduru, Stanley, Pope, & Weerasooriya, 2016). The primary cause of scale is calcium carbonate precipitation which occurs when carbonate alkali from ASP injection mixes with calcium ions from formation brine. Since oil reservoirs are by nature heterogeneous this is often largely an unavoidable phenomenon and can be managed by scale squeezes and acid clean ups, and/or continuous capillary injection of scale-inhibitors and/or weak acids. ASP pilots have successfully managed scale, but at a cost. MEA alkali produces significantly less carbonate in the injection solution (the only carbonate is formed from bicarbonate present in injection water). It has not yet been quantified, but it is highly likely than MEA alkali will lead to significantly less scale formation in production wells than sodium carbonate-based formulations. Further work needs to be performed but MEA/ACP formulations offer a low cost, low complexity, economically attractive chemical EOR option.

### REFERENCES

Berger, P. D., & Lee, C. H. (2006). Improved ASP Process Using Organic Alkali. SPE/DOE Symposium on Improved Oil Recovery. Tulsa. OK, USA: Society of Petroleum Engineers. doi:10.2118/99581-MS
Chang, L., Jang, S., Tagavifar, M., & Pope, G. A. (2018). Structure-Property Model for Microemulsion Phase Behavior. SPE Improved Oil Recovery Conference. Tulsa, OK, USA: Society of Petroleum Engineers. doi:10.2118/190153-MS
Farsi, S., Bouwmeester, R., Brewer, M., Southwick, J. G., Mahruqi, D., Karpan, V., & van Batenburg, D. (2020). Implementing ASP Without Water Softening in Reservoirs With Low Concentrations Of Divalent Ions. Muscat, Oman: Society of Petroleum Engineers. doi:https://doi.org/10.2118/200094-MS
Fortenberry, R., Kim, D. H., Nizamidin, N., Adkins, S., Arachchilage, G. W., Koh, H., Pope, G. A. (2015). Use of Cosolvents To Improve Alkaline/Polymer Flooding. SPE Journal, 20(2), 255-266. doi:https://doi.org/10.2118/166478-PA
Gaillard, N., Giovannetti, B., Leblanc, T., Thomas, A., Braun, O., & Favero, C. (2015). Selection of Customized Polymers to Enhance Oil Recovery from High Temperature Reservoirs. SPE Latin American and Caribbean Petroleum Engineering Conference. Quito, Ecuador: Society of Petroleum Engineers. doi:10.2118/207860-MS
Gaillard, N., Olivaud, M., Zaitoun, A., Ould-Metidji, M., Dupuis, G., Jouenne, S., Masalmeh, S. (2021). Injectivity and Propagation of Sulfonated Acrylamide-Based Copolymers in Low Permeability Carbonate Reservoir Cores in Harsh Salinity and Temperature Conditions: Challenges and Learnings from a Middle East Onshore Case Study. Abu Dhabi International Petroleum Exhibition & Conference. Abu Dhabi, UAE: Society of Petroleum Engineers. doi:10.2118/207860-MS
Hirasaki, G. J., Miller, C. A., & Puerto, M. (2011). Recent Advances in Surfactant EOR. SPE Journal, 16(04), 889-907. doi:10.2118/115386-PA
Jang, S. H., Liyanage, P. J., Tagavifar, M., Chang, L., Upamali, K. A., Lansakara-P, D., Pope, G. A. (2016). A Systematic Method for Reducing Surfactant Retention to Extremely Low Levels. SPE Improved Oil Recovery Conference. Tulsa, OK, USA: Society of Petroleum Engineers. doi:10.2118/179685-MS
Jang, S., Liyanage, P. J., Lu, J., Kim, D. H., Arachchilage, G. W., Britton, C., Pope, G. A. (2014). Microemulsion phase behavior measurements using live oils at high temperature and pressure. SPE Improved Oil Recovery Symposium. Tulsa, OK, USA: Society of Petroleum Engineers. doi:10.2118/169169-MS
Karpan, V., Volokitin, Y., Shuster, M., Tigchelaar, W., Chmuzh, I., Koltsov, I., Skripkin, A. (2014). West Salym ASP pilot: Project Front-End Engineering. SPE Improved Oil Recovery Symposium. Tulsa, OK, USA: Society of Petroleum Engineers. doi:https://doi.org/10.2118/169157-MS
Levitt, D., Jackson, A., Heinson, C., Britton, L. N., Malik, T., Dwarakanath, V., & Pope, G. A. (2009). Identification and Evaluation of High-Performance EOR Surfactants. SPE Reservoir Evaluation & Engineering, 12(02), 243-253. doi:10.2118/100089-PA
Liu, S., Zhang, D., Yan, W., Puerto, M., Hirasaki, G. J., & Miller, C. A. (2008). Favorable Attributes of Alkaline-Surfactant-Polymer Flooding. SPE Journal, 13(01), 5-16. doi:10.2118/99744-PA
Lu, J., Britton, C., Solairaj, S., Solairaj, S., Liyanage, P. J., Kim, D. H., Pope, G. A. (2014). Novel Large-Hydrophobe Alkoxy Carboxylate Surfactants for Enhanced Oil Recovery. SPE Journal, 19(6), 1024-1034. doi:10.2118/154261-PA
Nelson, R. C. (1983). The Effect of Live Crude on Phase Behavior and Oil-Recovery Efficiency of Surfactant Flooding Systems. SPE Journal, 23(03), 501-510. doi:10.2118/10677-PA
Nelson, R., Lawson, J. B., Thigpen, D., & Stegemeier, G. (1984). Cosurfactant-Enhanced Alkaline Flooding. SPE Enhanced Oil Recovery Symposium. Tulsa, OK, USA: Society of Petroleum Engineers. doi:https://doi.org/10.2118/12672-MS
Pandey, A., Koduru, N., Stanley, M., Pope, G. A., & Weerasooriya, U. P. (2016). Results of ASP Pilot in Mangala Field: A Success Story. SPE Improved Oil Recovery Conference. Tulsa, OK, USA: Society of Petroleum Engineers. doi:https://doi.org/10.2118/179700-MS
Qing, W. (2010). Processing high TAN crude: part I. Petroleum Technology Quarterly, pp. 35-43. Retrieved from www.digitalrefining.com/article/1000524
Rajapaksha, S. P., Britton, C., McNeil, R. I., Kim, D. H., Unomah, M., Kulawardana, E. U., Pope, G. A. (2014). Restoration of Reservoir Cores to Reservoir Condition before Chemical Flooding Tests. SPE Improved Oil Recovery Symposium. Tulsa, OK, USA: Society of Petroleum Engineers. doi:https://doi.org/10.2118/169887-MS
Schumi, B., Clemens, T., Wegner, J., Ganzer, L., Kaiser, A., Hincapie, R., & Leitenmueller, V. (2019). Alkali/Cosolvent/Polymer Flooding of High-TAN Oil: Using Phase Experiments, Micromodels, and Corefloods for Injection-Agent Selection. SPE Reservoir Evaluation & Engineering, 23(02), 463-479. doi:https://doi.org/10.2118/195504-PA
Southwick, J. G., Brewer, M. L., Pieterse, S. G., & Batenburg, D. W. (2018). Ammonia as Alkali for High Acid Number Oils. SPE EOR Conference at Oil and Gas West Asia. Muscat, Oman: Society of Petroleum Engineers. doi:https://doi.org/10.2118/190470-MS
Southwick, J. G., Pol, E. v., Rijn, C. H., Batenburg, D. W., Boersma, D. M., Svec, Y., Raney, K. H. (2016). Ammonia as Alkali for Alkaline/Surfactant/Polymer Floods. SPE Journal, 21(01), 010-021. doi:https://doi.org/10.2118/169057-PA
Southwick, J. G., Svec, Y., Chilek, G., & Shahin, G. T. (2012). Effect of Live Crude on Alkaline/Surfactant Polymer Formulations: Implications for Final Formulation Design. SPE Journal, 17(02), 352-361. doi:10.2118/135357-PA
Southwick, J., Brewer, M., van Batenburg, D., Pieterse, S., Bouwmeester, R., Mahrugi, D., Mjeni, R. (2020). Ethanolamine as Alkali for Alkali Surfactant Polymer Flooding—Development of a Low-Complexity Field Implementation Concept. SPE Improved Oil Recovery Conference. Virtual: Society of Petroleum Engineers. doi:https://doi.org/10.2118/200432-MS
Upamali, K. A., Liyanage, P. J., Jang, S., Shook, E., Weerasooriya, U. P., & Pope, G. A. (2018). New Surfactants and Cosolvents Increase Oil Recovery and Reduce Cost. SPE Journal, 23(06), 2202-2217. doi:https://doi.org/10.2118/179702-PA
Volokitin, Y., Shuster, M., Karpan, V., Koltsov, I., Mikhaylenko, E., Bondar, M. Y., . . . Brewer, M. L. (2018). Results of Alkaline-Surfactant-Polymer Flooding Pilot at West Salym Field. SPE EOR Conference at Oil and Gas West Asia. Muscat, Oman: Society of Petroleum Engineers. doi:https://doi.org/10.2118/190382-MS
Winsor, P. A. (1954). Solvent Properties of Amphiphilic Compounds (Vol. 68). London, UK: Butterworths Scientific Publ. Ltd. doi:10.1002/ange.19560681521

### Example 3: Glycerol-Based Surfactants/Co-Solvents for Chemical EOR Applications

### Oil Properties and Brine Compositions

Active oil gravity 15.3°API, oil viscosity ˜60 cP at 21° C.

Compositions shown in Table 8.

TABLE 8
Synthetic Brines for Experiments

| Ion | ppm | ppm | ppm |
|---|---|---|---|
| HCO₃_{^{−}} | 1,900 | 146 | 112 |
| Na⁺ | 8,150 | 945 | 9 |
| K⁺ | 51 | 2 | 2 |
| Cl⁻ | 11,484 | 1,374 | 1,599 |
| SO₄²⁻ | 36 | 0 | 0 |
| Total | 21,621 | 2,466 | 2,814 |
| Dissolved Solids | | | |
| [ppm TDS] | | | |

### Phase Behavior Results

The results of the formulation 1; 1% MEA and 0.5% glycerine-30PO-35EO-OH, mixing Brine A and B at 21° C. are shown in FIG. 22-23. FIG. 22 show the the activity map constructed from the data. FIG. 23 shows the images of the phase behavior tubes, where optimal salinity, e.g., salinity of lowest IFT Winsor type III, is identified by red arrows. The results demonstrate that 10% oil show low to ultra-low (UL) IFT type III microemulsion at 23-25k ppm TDS, 30% oil show low to UL IFT type III microemulsion at 21-23k ppm TDS, 50% oil shows low to UL IFT type III microemulsion at 16-18k ppm TDS, Type I shows low to UL IFT with mixing, and PD shows classical behavior with UL IFT type III.

The images of phase behavior tubes using a formulation dilution with WF: 1% MEA and 0.5% glycerine-30PO-35EO-OH, mixing Brine A at 21° C. are shown in FIG. 24, where optimal salinity, e.g., salinity of lowest IFT Winsor type III, is identified by red arrows.

The images of phase behavior tubes using a formulation dilution with WF: 1% MEA, 0.15% ABS, and 0.15% glycerine-30PO-10EO-OH, mixing Brine A at 21° C. are shown in FIG. 25, where optimal salinity, e.g., salinity of lowest IFT Winsor type III, is identified by red arrows.

The results of the ASP formulation; 1% MEA, 0.15% ABS, and 0.15% glycerine-30PO-35EO-OH with Brine A at 21° C. are shown in FIG. 26-27. FIG. 26 shows the activity map constructed from the data. FIG. 27 shows the images of the phase behavior tubes, where optimal salinity, e.g., salinity of lowest IFT Winsor type III, is identified by red arrows. The results demonstrate that 10% oil shows low to UL IFT type III microemulsion at 30-35k ppm TDS (better than formulation 1 for 10% oil), 30% oil shows low to UL IFT type III microemulsion at 22-28k ppm TDS, 50% oil shows low to UL IFT, and type III microemulsion at 9-15k ppm TDS. The slope of the activity map is steeper than formulation 1.

The images of phase behavior tubes using a formulation dilution with WF: 1% MEA, 0.15% ABS, and 0.15% glycerine-30PO-10EO-OH, mixing Brine A at 21° C. are shown in FIG. 28, where optimal salinity, e.g., salinity of lowest IFT Winsor type III, is identified by red arrows.

The images of phase behavior tubes using a formulation dilution with PD: 1% MEA, 0.15% ABS, and 0.15% glycerine-30PO-10EO-OH, mixing Brine A at 21° C. are shown in FIG. 29, where optimal salinity, e.g., salinity of lowest IFT Winsor type III, is identified by red arrows. FIG. 29A shows PD: soft Brine B (2.4k ppm). FIG. 29B shows PD: mix of brine A+B (10k ppm).

### Example 4: Emulsion Polymer—Mineral Oil Droplet Size Reduction: Improved Dispersion for Improved Injectivity

Reference solution: 2250 ppm emulsion polymer, 360 ppm TDA-8EO (assume 8% in original stock), and 1800 ppm mineral oil (assume ˜40% in original stock).

FIGS. 31-37 show results of studies performed to identify formulations with better filtration ratio with 1 μm polycarbonate filter compared to the reference solution. FIGS. 38-45 show results of studies performed to identify formulations that filter 200 mL faster than the reference solution using a 1 μm polycarbonate filter. FIGS. 46-53 show results of studies performed to identify formulations that filter greater volume than the reference solution at 2500 seconds with a 0.65 μm nitrocellulose filter. FIGS. 54-61 show results of studies performed to identify formulations that show relatively homogenous without rapid settling compared to the reference solution.

### Results for Gly-4PO

The Gly-4PO shows filtration ratio with 1 μm polycarbonate filter of 1.06 (FIG. 33) compared to baseline reference solution of 1.11 (FIG. 31), filtration speed of approximately 650 seconds compared to baseline reference solution of approximately 750 seconds, and filtration volume of 160 mL (FIG. 48) compared to baseline reference solution of approximately 115 mL (FIG. 46).

The Gly-4PO and the referenced solution (FIG. 54) were similar with respect to the settling properties. The emulsion polymer was compared to the referenced solution to assay settling properties such as relatively homogenous without rapid settling.

### Results for 2EH-2PO-10EO

The 2EH-2PO-10EO shows filtration ratio with 1 μm polycarbonate filter of 1.03 compared to baseline reference solution of 1.11, filtration speed of approximately 925 seconds (FIG. 43) compared to baseline reference solution of approximately 750 seconds (FIG. 31), and filtration volume of 180 mL compared to baseline reference solution of approximately 115 mL (FIG. 46).

The 2EH-2PO-10EO was compared to the referenced solution (FIG. 54) to assay settling properties such as relatively homogenous without rapid settling. The 2EH-2PO-10EO showed faster segregation (FIG. 59) compared to the referenced solution (FIG. 46).

### Results for TDA-8EO

The TDA-8EO shows filtration ratio with 1 μm polycarbonate filter of 1.11 compared to baseline reference solution of 1.11 (FIG. 31), filtration speed of approximately 450 seconds (FIG. 38) compared to baseline reference solution of approximately 750 seconds (FIG. 31), filtration volume of 165 mL compared to baseline reference solution of approximately 115 mL (FIG. 46).

The TDA-8EO and the referenced solution were similar with respect to the settling properties. The TDA-8EO was compared to the referenced solution to assay settling properties such as relatively homogenous without rapid settling (FIG. 46).

1. A compound defined by Formula I 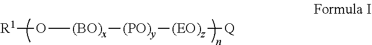
wherein
BO represents —CH₂—CH(ethyl)-O— or —CH₃CH(O—)CH₃;
PO represents —CH₂—CH(methyl)-O—;
EO represents —CH₂—CH₂—O—;
R¹ represents a C₁-C₈ alkyl group (e.g., a C₁-C₆ alkyl group) or a cyclic structure derived from alkoxylation of an alkyl monoglucoside, an alkyl polyglucoside, a monosaccharide, a disaccharide, or a polysaccharide;
n is an integer from 2 to 6;
Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation;
x is an integer from 0 to 15;
y is an integer from 11 to 60; and
z is an integer from 0 to 100.

2. The compound of claim 1, wherein n is an integer from 3 to 6.

3. The compound of claim 1, wherein y is an integer from 11 to 40.

4. The compound of claim 1, wherein y is from 11 to 20.

5. The compound of claim 1, wherein R¹ is a C₁-C₆ alkyl group.

6. The compound of claim 1, wherein when Q is hydrogen, z is at least 6.

7. The compound of claim 1, wherein Q is —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH; and M⁺ is a cation.

8. The compound of claim 1, wherein z is greater than 1.

9. The compound of claim 1, wherein z is an integer from 5 to 75, such as from 5 to 50 or from 5 to 35.

10. The compound of claim 1, wherein x is 0.

11. The compound of claim 1, wherein n is 3.

12. The compound of claim 11, wherein the compound is defined by Formula II below 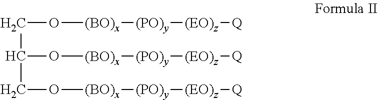
wherein
BO represents —CH₂—CH(ethyl)-O— or —CH₃CH(O—)CH₃;
PO represents —CH₂—CH(methyl)-O—;
EO represents —CH₂—CH₂—O—;
Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation;
x is an integer from 0 to 15;
y is an integer from 11 to 60; and
z is an integer from 0 to 100.

13. The compound of claim 1, wherein n is 4.

14. The compound of claim 13, wherein the compound is defined by Formula III below 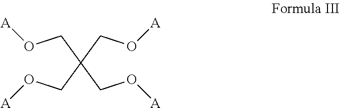
wherein
A represents —(BO)ₓ—(PO)_{y}-(EO)_{z}-Q
BO represents —CH₂—CH(ethyl)-O— or —CH₃CH(O—)CH₃;
PO represents —CH₂—CH(methyl)-O—;
Q is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation;
x is an integer from 0 to 15;
y is an integer from 11 to 60; and
z is an integer from 0 to 100.

15-37. (canceled)

38. A compound defined by Formula IX 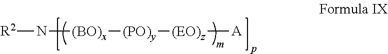
wherein
BO represents —CH₂—CH(ethyl)-O— or —CH₃CH(O—)CH₃;
PO represents —CH₂—CH(methyl)-O—;
EO represents —CH₂—CH₂—O—;
R² is absent, —((BO)ₓ—(PO)_{y}-(EO)_{z}-)ₘ-A, or —CH₂R³;
R³ is C₁-C₁₀ alkoxy, aryloxy, or —C(O)O⁻M⁺, or —C(O)OH;
m is an integer from 1 to 3;
p is an integer from 2 to 3;
A is hydrogen, —SO₃M⁺, —SO₃H, CH₂CH(OH)CH₂—SO₃M⁺, CH₂CH(OH)CH₂—SO₃H, —CH₂C(O)O⁻M⁺, or —CH₂C(O)OH;
M⁺, when present, is a cation;
x is an integer from 0 to 15;
y is an integer from 11 to 60; and
z is an integer from 0 to 100,
wherein when R² is absent, p is 3.

39. A composition comprising a compound of claim 1 and water.

40. An emulsion comprising the composition of claim 39 and unrefined petroleum.

41. A method of displacing an unrefined petroleum material in contact with a solid material, said method comprising:
contacting the unrefined petroleum material with the compound of claim 1 or the composition of claim 39, wherein the unrefined petroleum material is in contact with the solid material; and
allowing the unrefined petroleum material to separate from the solid material, thereby displacing the unrefined petroleum material in contact with the solid material.

42. A polymer composition comprising:
(1) a liquid polymer (LP) composition comprising: one or more synthetic (co)polymers, an inversion package comprising a compound of claim 1, optionally one or more hydrophobic liquids, and optionally one or more surfactants; or
(2) a powder polymer suspended in an inversion package comprising a compound of claim 1—and optionally a water soluble solvent, wherein the powder polymer and the inversion package are present at a weight ratio of powder polymer to inversion package of from 20:80 to 80:20.

43. A method of preparing an inverted polymer solution comprising providing a liquid polymer composition of claim 42;
inverting the polymer composition in an aqueous fluid to provide an inverted polymer solution having a concentration of synthetic (co)polymer of from 50 to 50,000 ppm;
wherein the inverted polymer solution is used in an enhanced oil recovery (EOR) operation.
